(19) United States
(12) Patent Application Publication
(10) Pub. No.: US 2014/0128901 A1
(43) Pub. Date: May 8, 2014

(54) IMPLANT FOR ANEURYSM TREATMENT

(71) Applicants: Kevin Kang, San Jose, CA (US); Jeffrey Madsen Pappas, Santa Clara, CA (US)

(72) Inventors: Kevin Kang, San Jose, CA (US); Jeffrey Madsen Pappas, Santa Clara, CA (US)

(21) Appl. No.: 13/971,741

(22) Filed: Aug. 20, 2013

Related U.S. Application Data
(60) Provisional application No. 61/853,323, filed on Apr. 3, 2013.
(60) Provisional application No. 61/853,246, filed on Apr. 1, 2013.
(60) Provisional application No. 61/852,516, filed on Mar. 18, 2013.
(60) Provisional application No. 61/796,166, filed on Nov. 5, 2012.

(51) Int. Cl. A61B 17/12

(52) U.S. Cl. CPC A61B 17/12113
USPC 606/194

(57) ABSTRACT

Implants and methods used for the treatment of aneurysms are disclosed. More particularly, embodiments of an implant having a modular pattern are disclosed for the treatment of cerebral aneurysms to prevent intracranial hemorrhage and aneurysm rupture.

This application claims the benefit of U.S. Provisional Patent Application Nos. 61/853,323 filed Apr. 3, 2013, 61/853,246 filed Apr. 1, 2013, 61/852,516 filed Mar. 18, 2013, and 61/796,166 filed Nov. 5, 2012, and this application hereby incorporates herein by reference those provisional patent applications.

### BACKGROUND

1. Field

The present invention relates to implants used for the treatment of aneurysms. More particularly, embodiments of the present invention relate to an implant that treats cerebral aneurysms by preventing intracranial hemorrhage and aneurysm rupture.

2. Background Information

Aneurysms are pathological bulges in vascular anatomies, typically caused either by disease or weakening of a vessel wall. As shown in FIG. 1, an aneurysm 100 may occur in the cerebral vessels 102 of a patient 104, such as in the vertebral, basilar, middle cerebral, posterior cerebral, or internal carotid arteries. Typically, cerebral vessels include vessel diameters in a range of between about 1.5 to 5.5 mm (3.5 mm average). FIG. 2A illustrates a cerebral aneurysm 100 classified as a saccular aneurysm, which includes an aneurysm sac 200 joined with a portion of a vessel 202 at an aneurysm gate 204. Usually, aneurysm gates 204 vary between about 4 to 5.5 mm in diameter and aneurysm sacs 200 commonly include a diameter in a range of about 8 to 10 mm. That is, the aneurysm sac diameter is commonly about twice the aneurysm gate diameter. However, saccular aneurysms may be wide-necked, as shown in FIG. 2B. Wide-necked aneurysms are characterized by an aneurysm gate 204 opening that roughly corresponds to a diameter of the aneurysm sac 200. FIG. 2C illustrates another classification of aneurysm, referred to as a fusiform aneurysm, which includes an aneurysm sac 200 tapering to an aneurysm gate 204 that generally involves the circumference of vessel 202. Unless an aneurysm is depressurized, the aneurysm may eventually rupture, leading to severe complications. For example, in the case of cerebral aneurysms, a ruptured aneurysm may lead to severe intracranial hemorrhage with associated loss of perception, loss of balance, or even death.

Numerous approaches have been developed to treat vascular aneurysms, including some minimally invasive techniques. For example, as illustrated in FIG. 3, an endovascular coiling procedure may be used to treat cerebral aneurysms. During an endovascular coiling procedure, a microcatheter may be intravascularly tracked to an aneurysm 100 site and one or more embolic coils 300 may be inserted into an aneurysm sac 200 to promote blood clotting that occludes and depressurizes the sac. Although this approach is intended to seal the aneurysm to prevent or reduce cerebral hemorrhage, in some cases the insertion of coils 300 may actually cause vessel rupture. Furthermore, in the case of wide-necked aneurysms, embolic coils may not be adequately retained and may protrude or migrate into the parent vessel, causing further complications.

Another approach to treating vascular aneurysms includes stenting across the aneurysm gate. For example, as shown in FIG. 4, a stent 400 may be delivered across an aneurysm gate to create and/or retain a thrombus 402 within the aneurysm sac 200 and thereby occlude and depressurize the aneurysm. The stent 400 may be deployed across the aneurysm gate before or after inserting an embolic coil into the aneurysm sac to form a thrombus. In a technique referred to as “jailing”, an embolic coil 300 may be placed in the aneurysm sac 200 and then held there by subsequent delivery of a stent 400. Alternatively, a stent 400 may be deployed across the aneurysm gate 204 and then a microcatheter may be tracked through the expanded stent 400 struts into the aneurysm sac 200 to insert an embolic coil 300 therein.

In alternative embodiments, a stent or stent graft may be used alone to act as a flow diverter that slows or prevents blood flow into the aneurysm with the goal of removing flow and pressure against an aneurysm sac. However, traditional stents and stent grafts often have a profile that prohibits their delivery into small cerebral vessels. Additionally, in the case of traditional stents, scaffolding area over the aneurysm gate may be insufficient to adequately divert blood flow from the aneurysm to depressurize the aneurysm sac. Furthermore, in the case of traditional stent grafts, there is a risk of inadvertently occluding blood vessels adjacent to the aneurysm.

The clinical conditions under which aneurysm treatment is performed may also impact treatment success. More specifically, controlled clotting of the aneurysm is often a goal of treatment, but in some cases clots migrate into a parent vessel and inadvertently occlude downstream vessel segments. Intravenously administered tissue plasminogen activator or local intra-arterial thrombolysis may be given to a patient to disrupt clots in the parent vessel and prevent downstream occlusions. However, the use of such drugs not only makes it more difficult to control clotting in the aneurysm, but may actually increase the risk of symptomatic intracranial hemorrhage associated with the aneurysm.

### SUMMARY OF THE DESCRIPTION

Implants used for treating aneurysms are disclosed. In an embodiment, a vascular implant is provided having an unexpanded state and an expanded state. The vascular implant may include a base section and an aneurysm section. The base section may have a plurality of base rings arranged along a longitudinal axis and the base section may be cylindrical in both the unexpanded state and the expanded state. The aneurysm section may have a plurality of aneurysm section holders extending longitudinally from the base section and an aneurysm pattern radially disposed between the plurality of aneurysm section holders. The aneurysm pattern may be substantially cylindrical in the unexpanded state and substantially non-cylindrical in the expanded state. In an embodiment, the aneurysm pattern includes a plurality of aneurysm arcs extending radially between the plurality of aneurysm section holders and one or more of the aneurysm arcs extend along a substantially circumferential path in the unexpanded state and extend along a substantially non-circumferential path in the expanded state. For example, in an embodiment, the aneurysm pattern includes a substantially bulbous contour in the expanded state. Alternatively, in an embodiment, the aneurysm pattern includes a substantially longitudinal cylindrical segment contour in the expanded state.

In an embodiment, the base section of the implant may include a proximal subsection and a distal subsection and the plurality of aneurysm section holders may extend longitudinally between the proximal subsection and the distal subsection. The aneurysm section may further include an aneurysm connector extending longitudinally between the proximal subsection and the distal subsection. The aneurysm connector may be opposite of the plurality of aneurysm section holders from the aneurysm pattern.

In an embodiment, the implant may further include one or more aneurysm marker holders in each of the plurality of aneurysm section holders, and an aneurysm marker in each aneurysm marker holder. One or more of the aneurysm marker holders may be longitudinally spaced along respective aneurysm section holder and the aneurysm markers may include radiopaque markers.

In an embodiment, each base ring may include a plurality of base struts interconnected by a plurality of base joints and arranged in a ring pattern. In an embodiment, each base strut extends straightly between a respective pair of base joints. In another embodiment, each base strut undulates between a respective pair of base joints. The ring pattern of a first base ring may include a sawtooth pattern and the ring pattern of a second base ring adjacent to the first base ring may include a sawtooth pattern, such that the sawtooth pattern of the second base ring is inverted relative to the sawtooth pattern of the first base ring.

In an embodiment, a plurality of base ring connectors interconnect adjacent base rings. The plurality of base ring connectors may interconnect adjacent base rings at radially staggered locations along a substantially helical path. The plurality of base rings may include a transition ring connected to the aneurysm section. The transition ring may interconnect an adjacent base ring with the plurality of aneurysm section holders and may also include more base joints than the adjacent base ring. In an embodiment, a transition ring connector interconnects the transition ring with the adjacent base ring, and the plurality of aneurysm section holders extend longitudinally from a plurality of base joints of the transition ring.

In an embodiment, a method of using the implant is provided, including advancing the vascular implant into a vessel segment in an unexpanded state with both the base section and the aneurysm pattern substantially cylindrical in the unexpanded state. The method may include aligning one or more aneurysm markers in each of the plurality of aneurysm section holders with an aneurysm gate. Furthermore, in an embodiment, the method includes deploying the vascular implant to an expanded state within the vessel segment at a site of an aneurysm, the aneurysm having an aneurysm sac adjoined to the vessel segment at the aneurysm gate. The base section of the vascular implant may be substantially cylindrical and the aneurysm pattern may be substantially non-cylindrical in the expanded state. For example, the aneurysm pattern may include a substantially bulbous contour bulging into the aneurysm sac in the expanded state. Alternatively, the aneurysm pattern may include a substantially longitudinal cylindrical segment contour collinear with a vessel wall of the vessel segment in the expanded state.

### BRIEF DESCRIPTION OF THE DRAWINGS

FIG. 1 is a pictorial view illustrating a patient with a cerebral aneurysm.

FIG. 2A is a detail view, taken from Detail A of FIG. 1, of a saccular aneurysm.

FIG. 2B is a detail view, taken from Detail A of FIG. 1, of a wide-necked aneurysm.

FIG. 2C is a detail view, taken from Detail A of FIG. 1, of a fusiform aneurysm.

FIG. 3 is a pictorial view of an aneurysm coil deployed inside of an aneurysm.

FIG. 4 is a pictorial view of a stent deployed across an aneurysm.

FIG. 5 is a perspective view of a vascular implant in an unexpanded state in accordance with an embodiment of the invention.

FIG. 6A-6B are perspective views of a vascular implant in various expanded states in accordance with an embodiment of the invention.

FIG. 7 is a side view of a vascular implant in an unexpanded state in accordance with an embodiment of the invention.

FIG. 8A-8C are cross-sectional views, taken about line A-A of FIG. 7, of a base section of a vascular implant transitioning from an unexpanded state to an expanded state in accordance with an embodiment of the invention.

FIG. 9A-9C are cross-sectional views, taken about line B-B of FIG. 7, of an aneurysm section of a vascular implant transitioning from an unexpanded state to an expanded state in accordance with an embodiment of the invention.

FIG. 10 is a cross-sectional view, taken about line B-B of FIG. 7, of an aneurysm section of a vascular implant in an expanded state in accordance with an embodiment of the invention.

FIG. 11A-11E are flat pattern illustrations of a vascular implant having various embodiments of aneurysm connectors in accordance with an embodiment of the invention.

FIG. 12A is a flat pattern illustration of a base section of a vascular implant having straight base struts in accordance with an embodiment of the invention.

FIG. 12B is a detail view, taken from Detail B of FIG. 12A, of a base ring connector region of a base section of a vascular implant in accordance with an embodiment of the invention.

FIGS. 13A-13B are flat pattern illustrations of alternative base ring connector regions of a base section of a vascular implant in accordance with an embodiment of the invention.

FIG. 14A is a flat pattern illustration of a base section of a vascular implant having undulating base struts with a triple-wave design in accordance with an embodiment of the invention.

FIG. 14B is a detail view, taken from Detail C of FIG. 14A, of an alternating base strut pattern of a base section of a vascular implant in accordance with an embodiment of the invention.

FIG. 15A is a flat pattern illustration of a base section of a vascular implant having undulating base struts with a quadruple-wave design in accordance with an embodiment of the invention.

FIG. 15B is a detail view, taken from Detail D of FIG. 15A, of a non-alternating base strut pattern of a base section of a vascular implant in accordance with an embodiment of the invention.

FIG. 16A is a flat pattern illustration of a zig-zag aneurysm pattern of a vascular implant in an expanded state in accordance with an embodiment of the invention.

FIG. 16B is a flat pattern illustration of a zig-zag aneurysm pattern of a vascular implant in an unexpanded state in accordance with an embodiment of the invention.

FIG. 17A is a flat pattern illustration of a parallelogram aneurysm pattern of a vascular implant in an expanded state in accordance with an embodiment of the invention.

FIG. 17B is a flat pattern illustration of a parallelogram aneurysm pattern of a vascular implant in an unexpanded state in accordance with an embodiment of the invention.

FIG. 18A is a flat pattern illustration of a transition ring of a vascular implant in accordance with an embodiment of the invention.

FIG. 18B is a detail view, taken from Detail E of FIG. 18A, of a transition ring connector of a vascular implant in accordance with an embodiment of the invention.

FIG. 19 is a side view of an end marker of a vascular implant in accordance with an embodiment of the invention.

FIG. 20A is a side view of an aneurysm marker near a medial location of an aneurysm section holder of a vascular implant in accordance with an embodiment of the invention.

FIG. 20B is a side view of an aneurysm marker near a base location of an aneurysm section holder of a vascular implant in accordance with an embodiment of the invention.

FIG. 21A-21J are flat pattern illustrations of numerous alternative embodiments of a vascular implant in accordance with an embodiment of the invention.

FIG. 22A-22G are flat pattern illustrations of numerous alternative embodiments of aneurysm patterns of a vascular implant in accordance with an embodiment of the invention.

FIG. 23 is a pictorial view of an intravascular access path to an aneurysm site in a patient.

FIG. 24A-24C are pictorial views of various stages of deployment of a vascular implant at an aneurysm site in accordance with an embodiment of the invention.

FIG. 25 is a schematic view showing a plurality of possible contours of an aneurysm pattern of a vascular implant deployed at an aneurysm site in accordance with an embodiment of the invention.

FIG. 26A-26D are pictorial views of a vascular implant deployed at a site of an aneurysm during various stages of aneurysm depressurization in accordance with an embodiment of the invention.

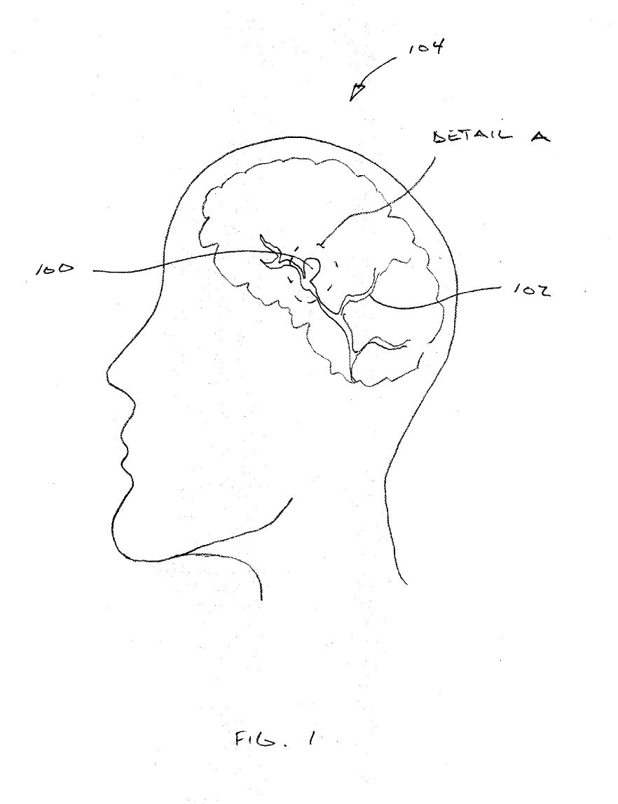

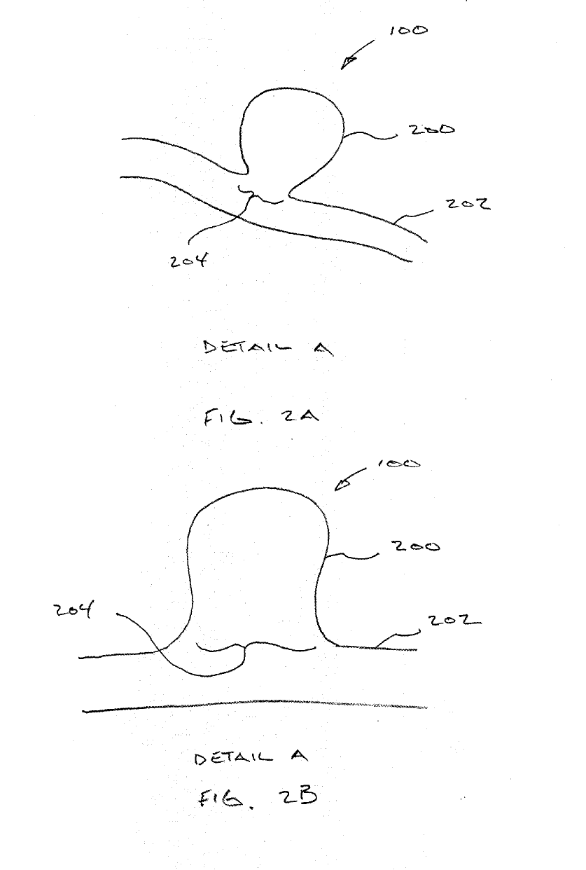

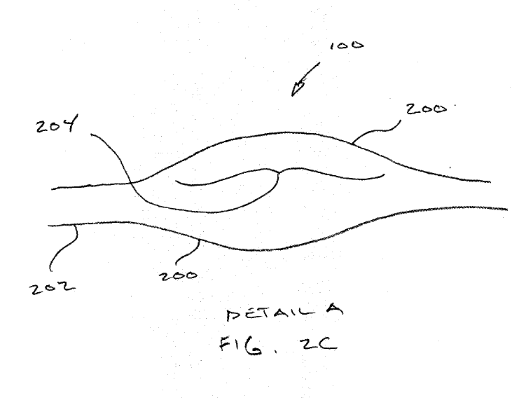

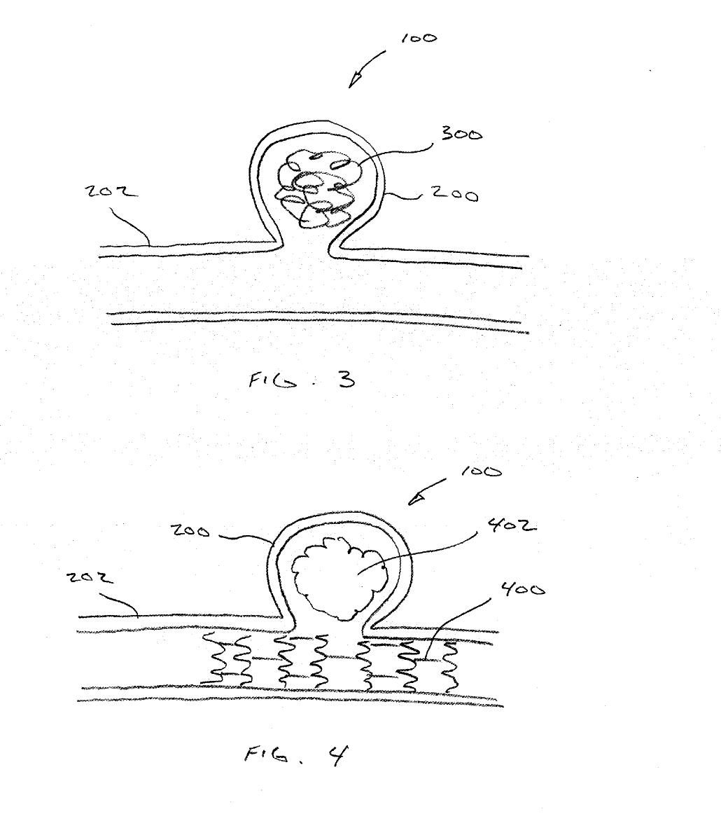

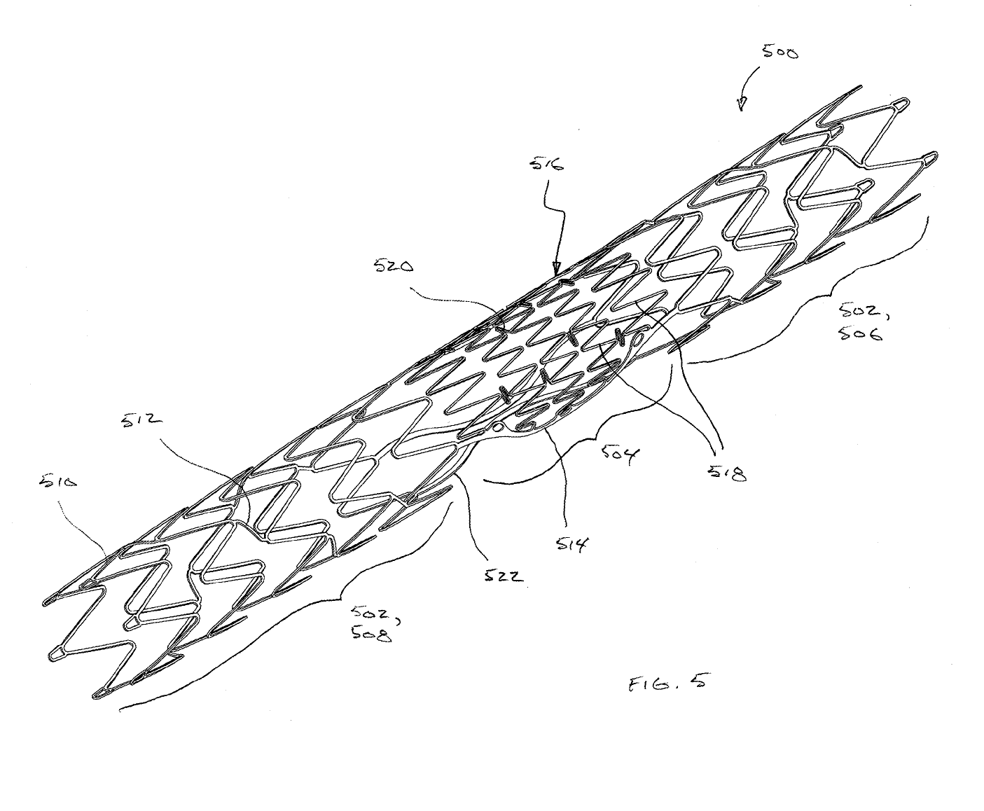

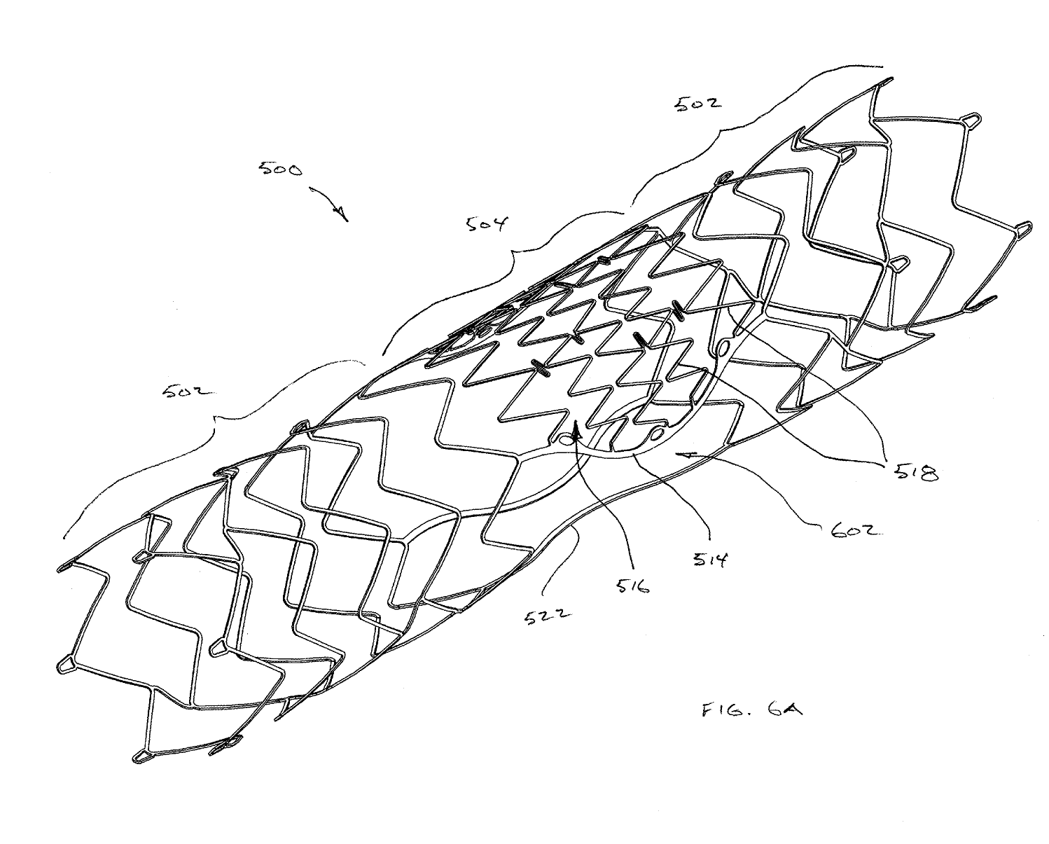

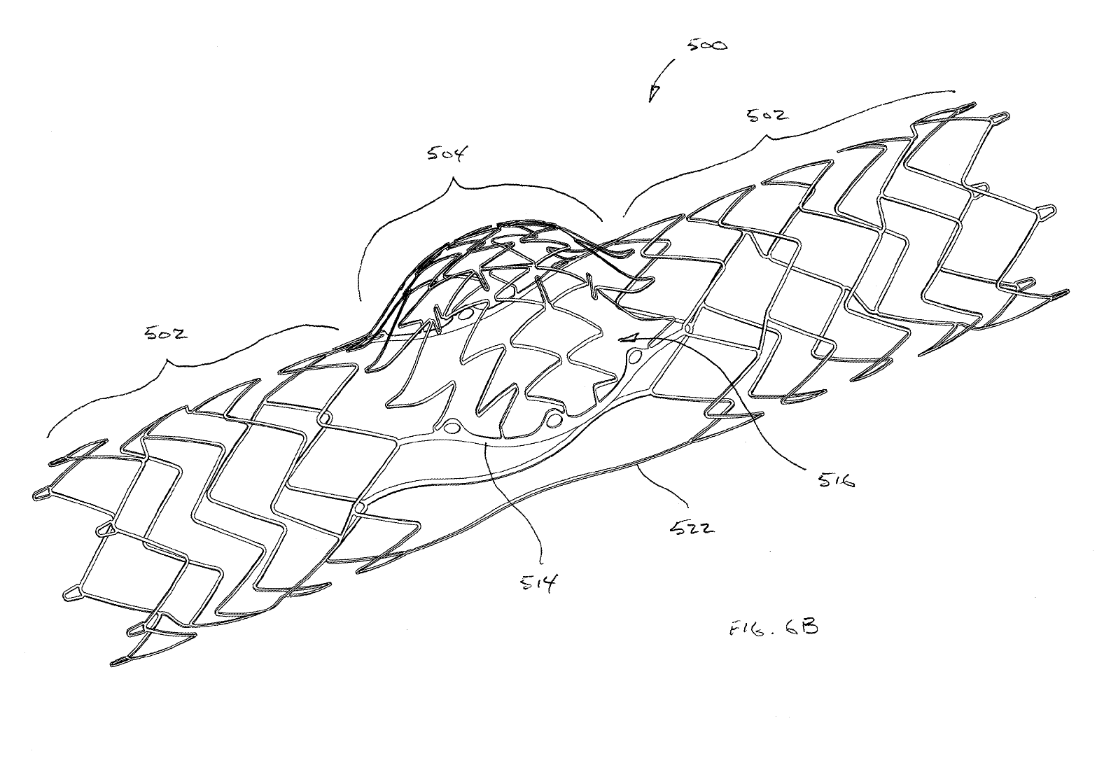

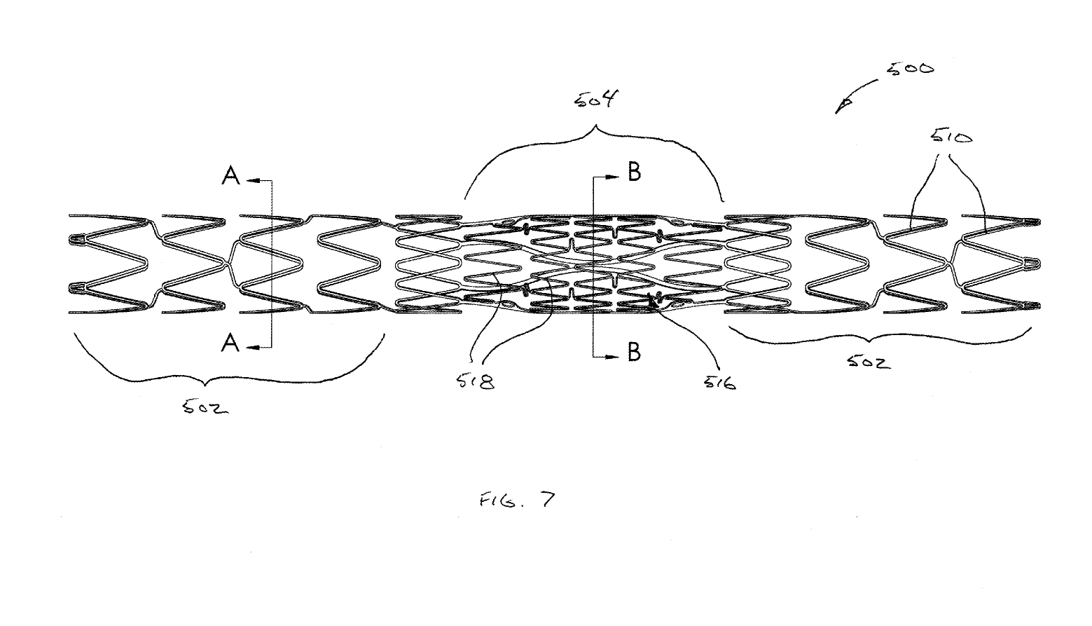

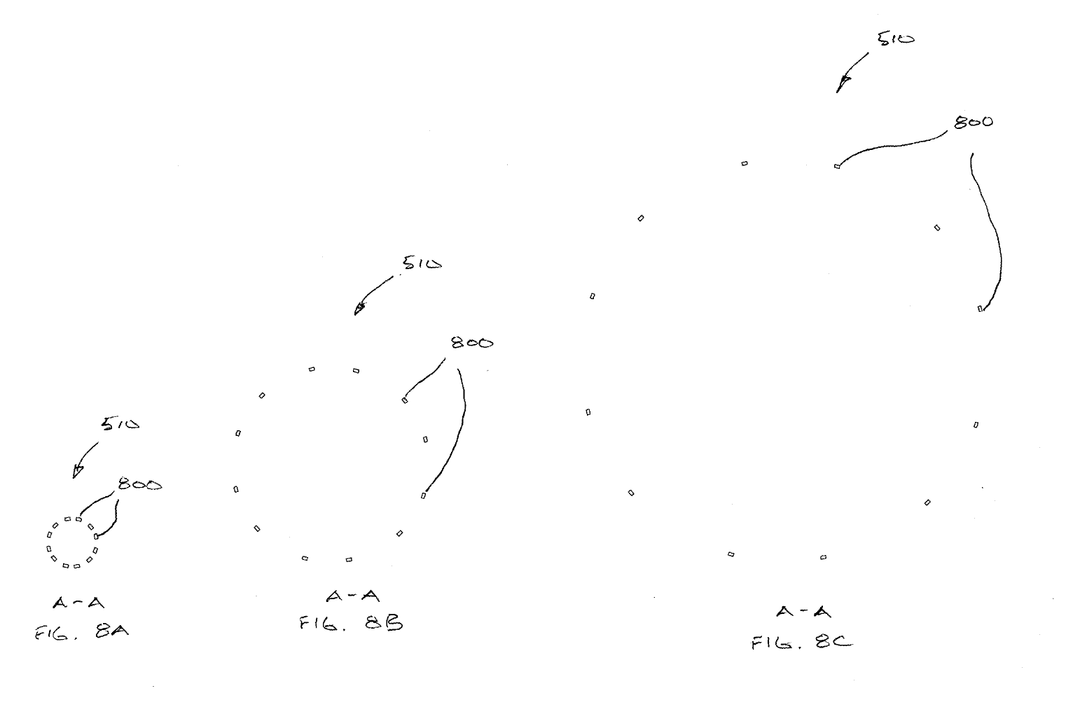

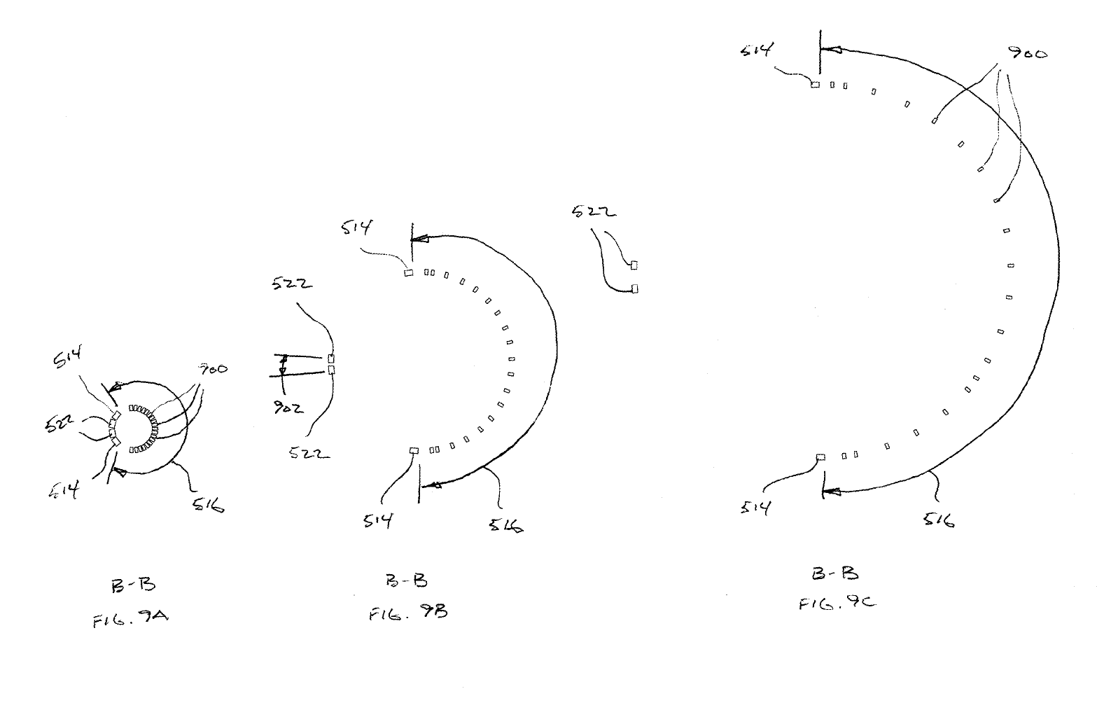

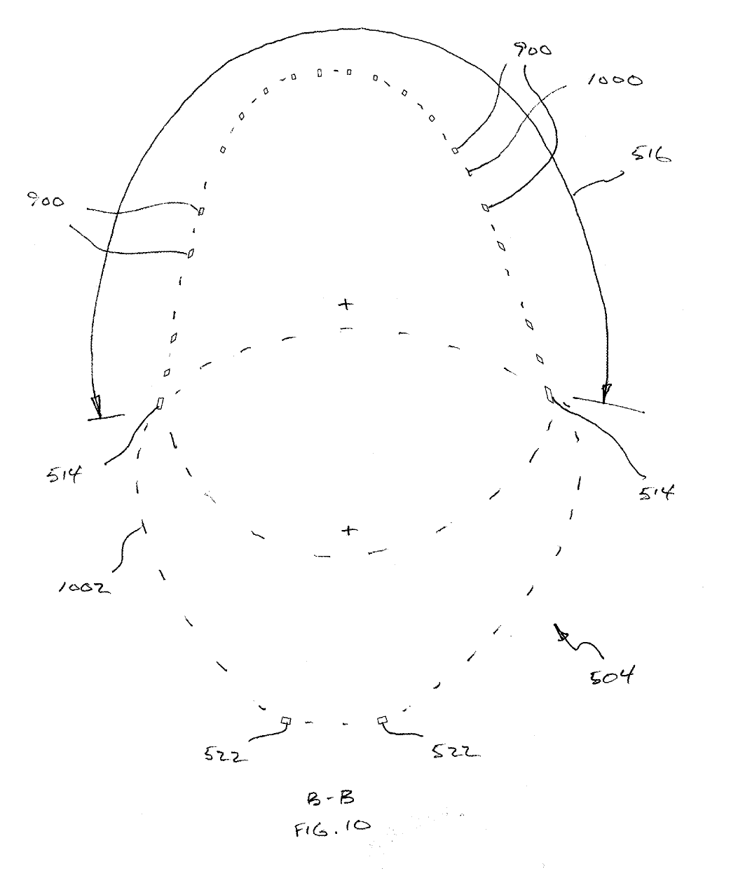

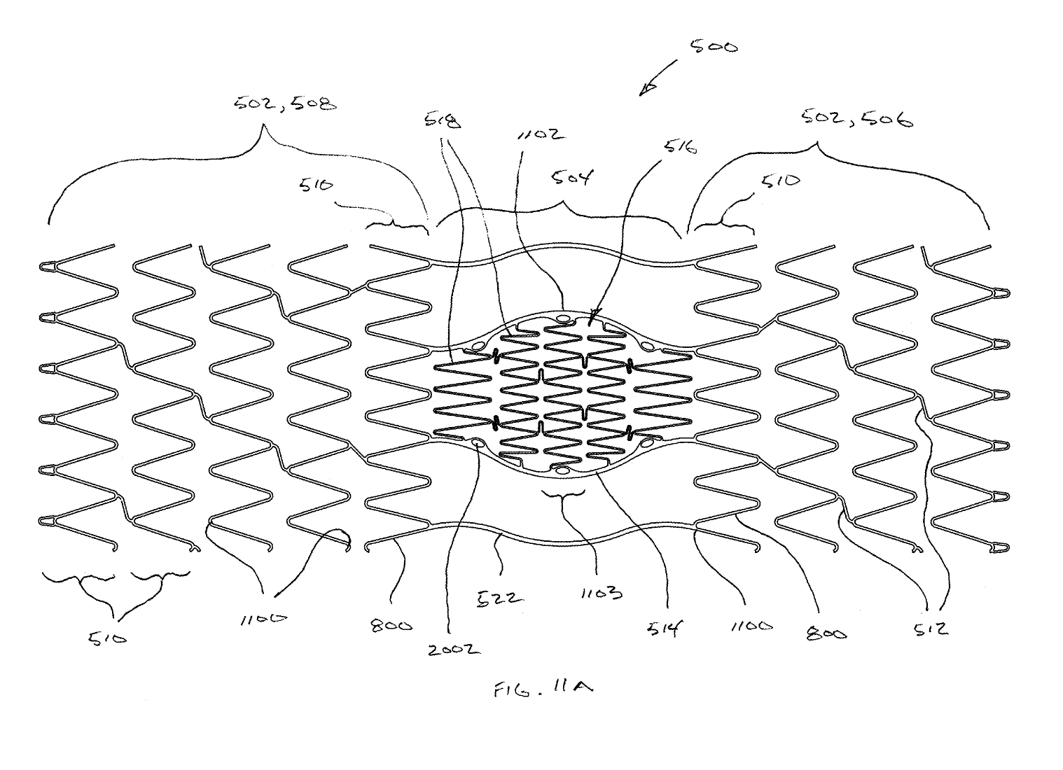

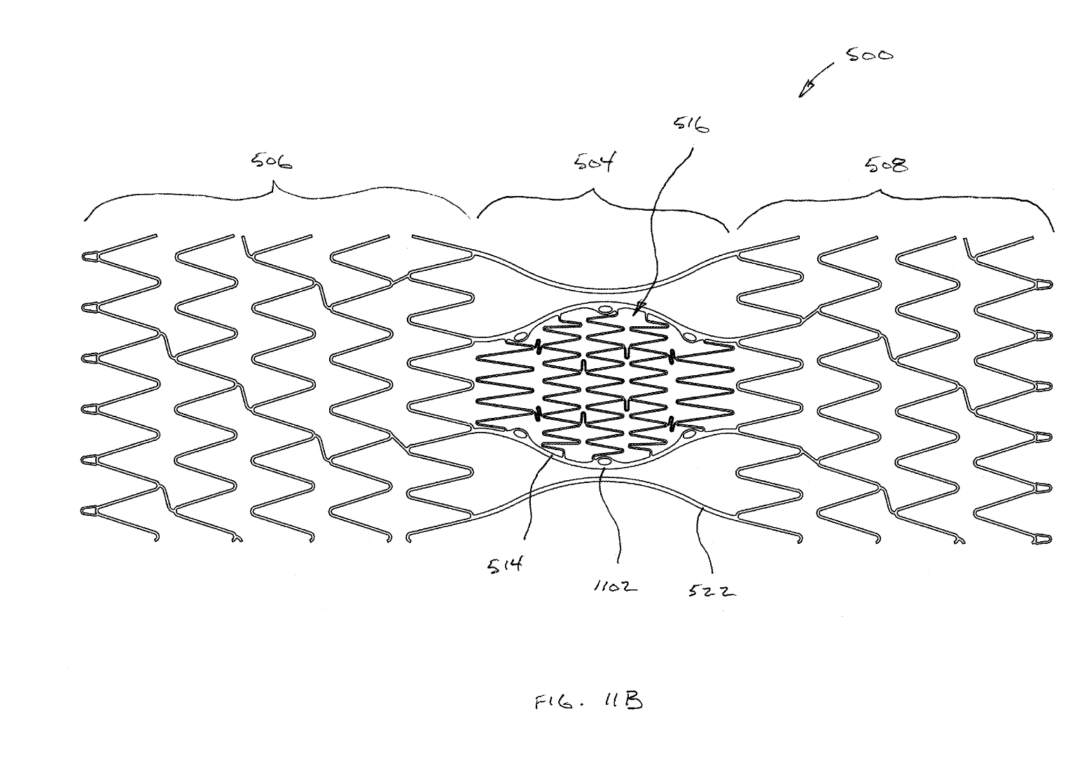

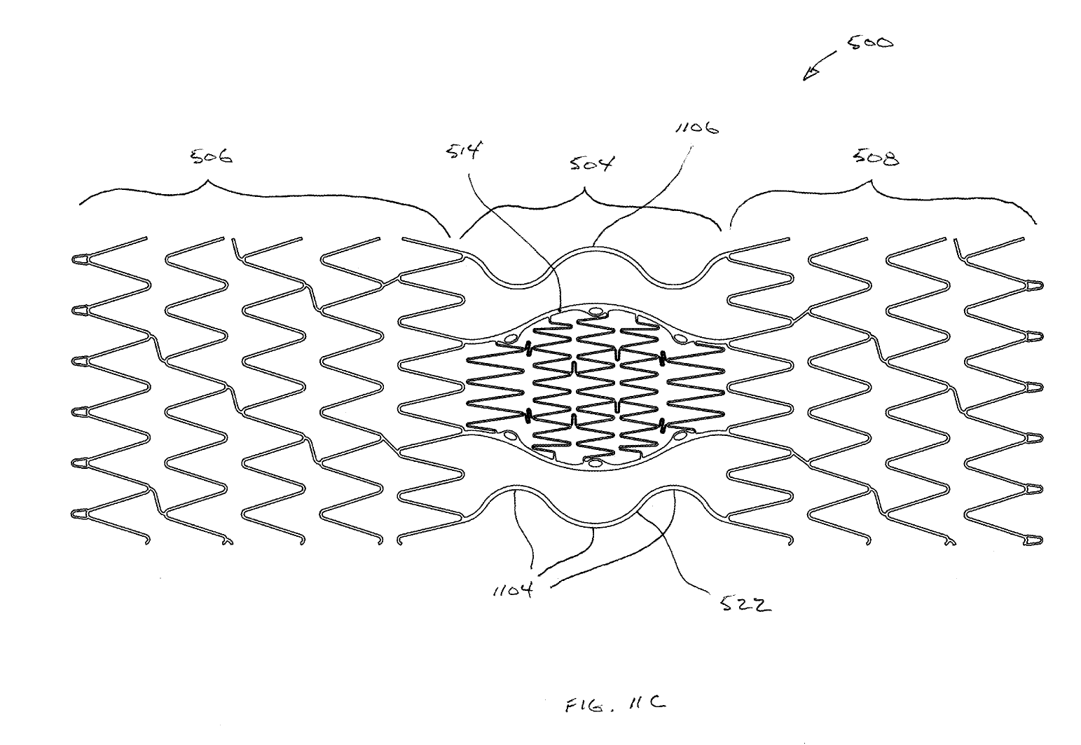

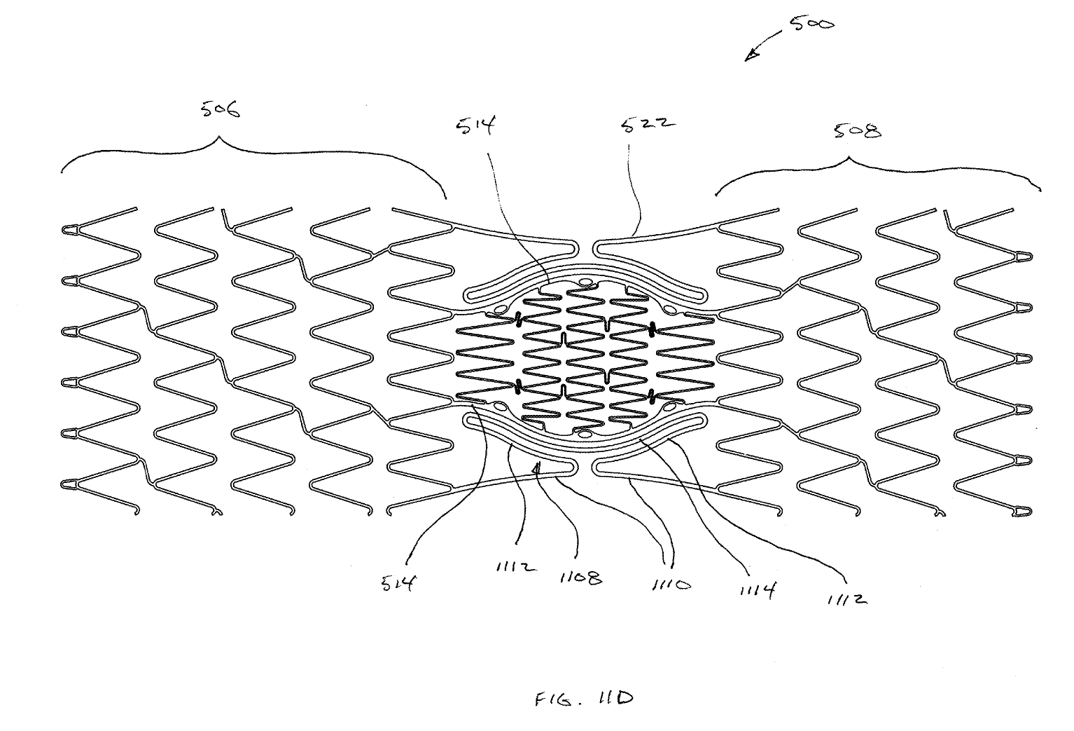

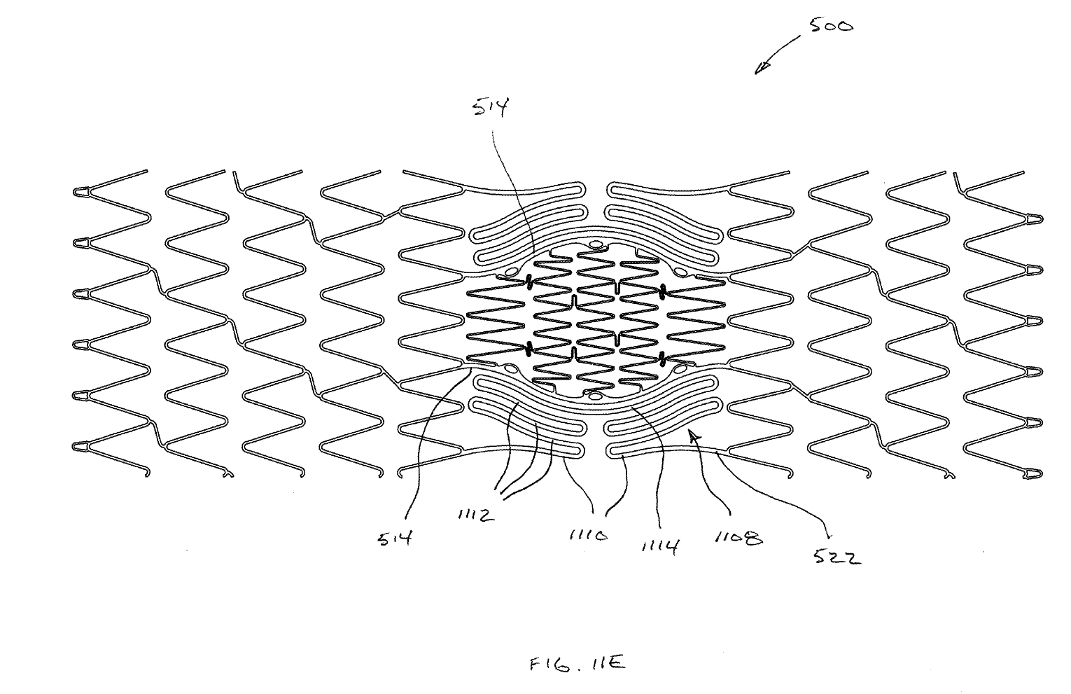

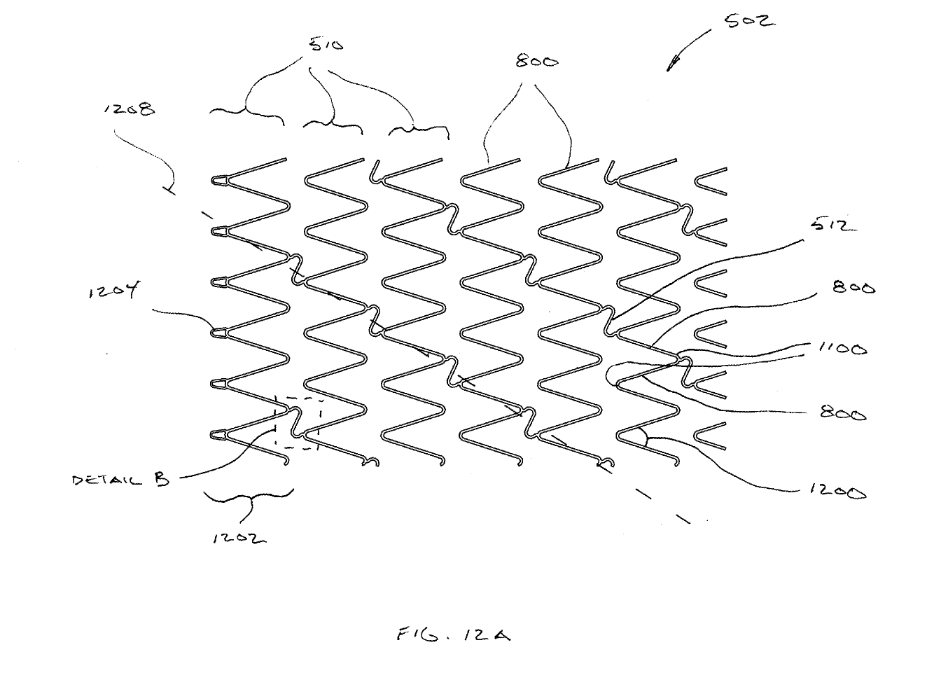

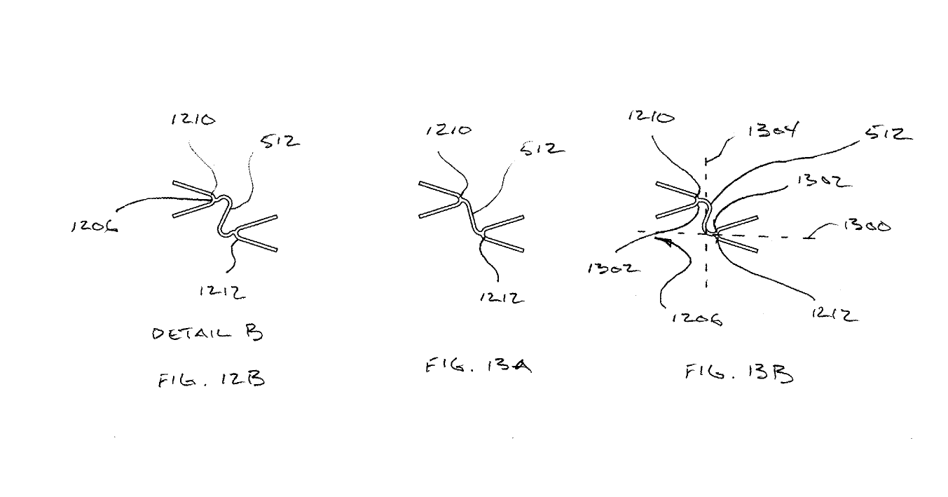

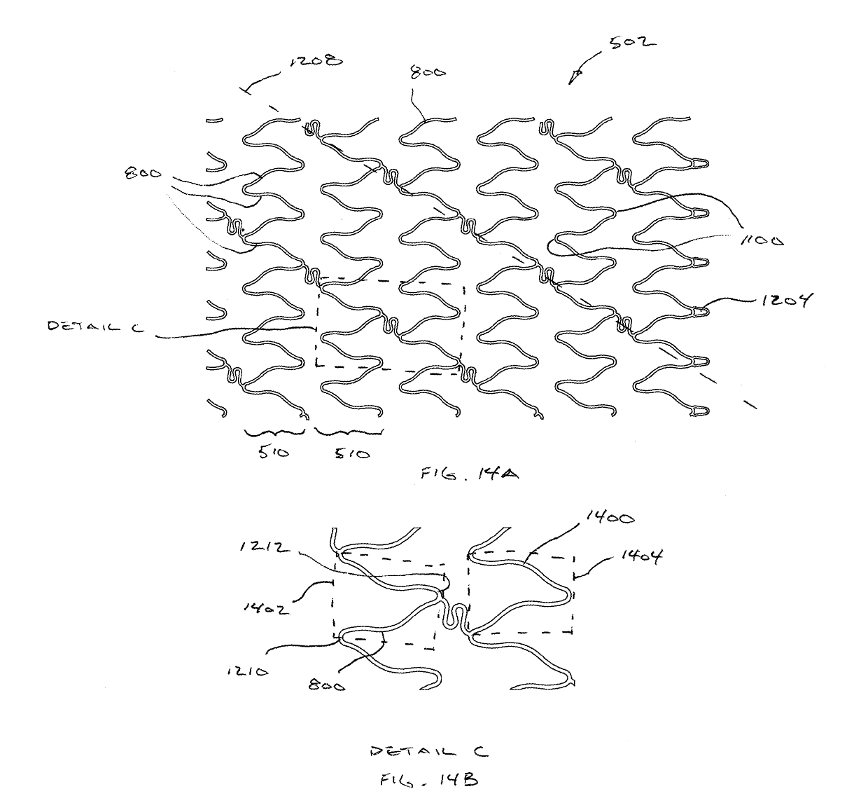

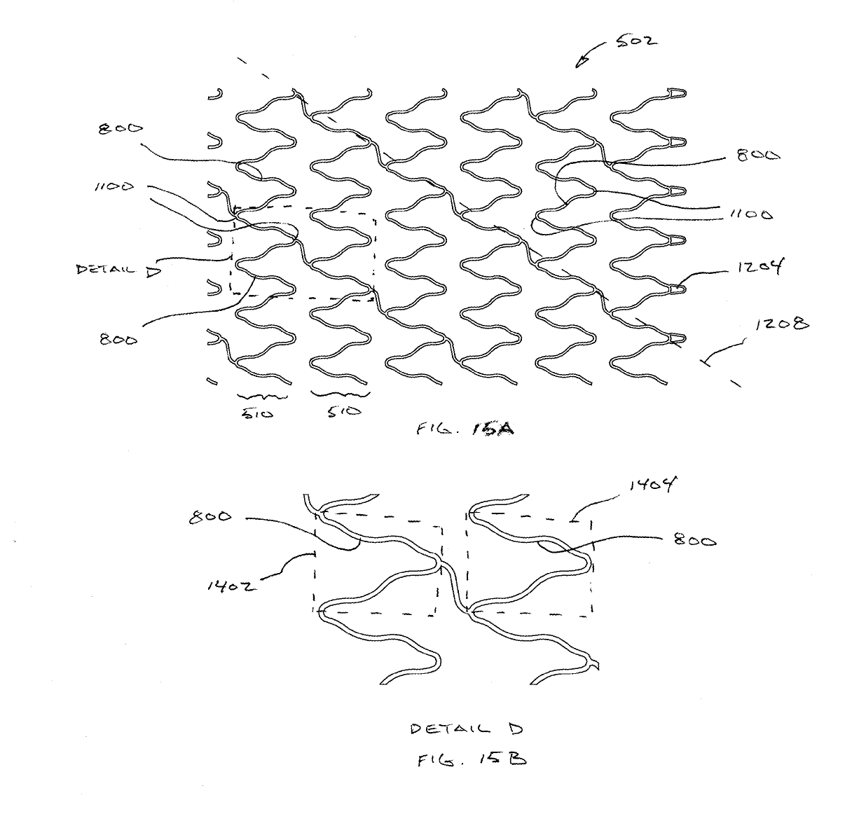

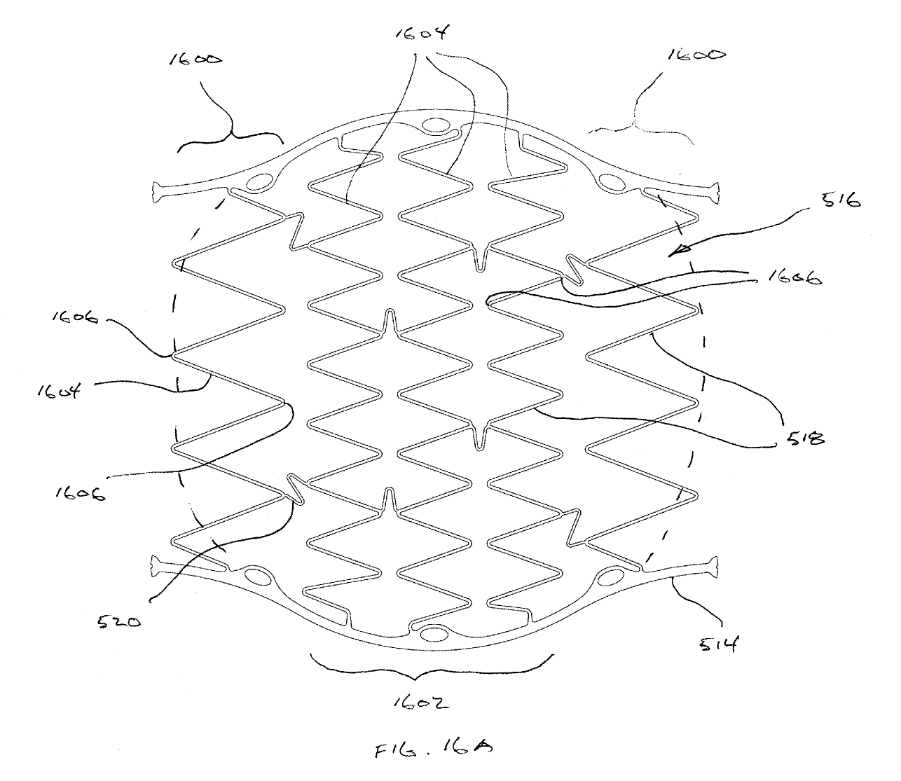

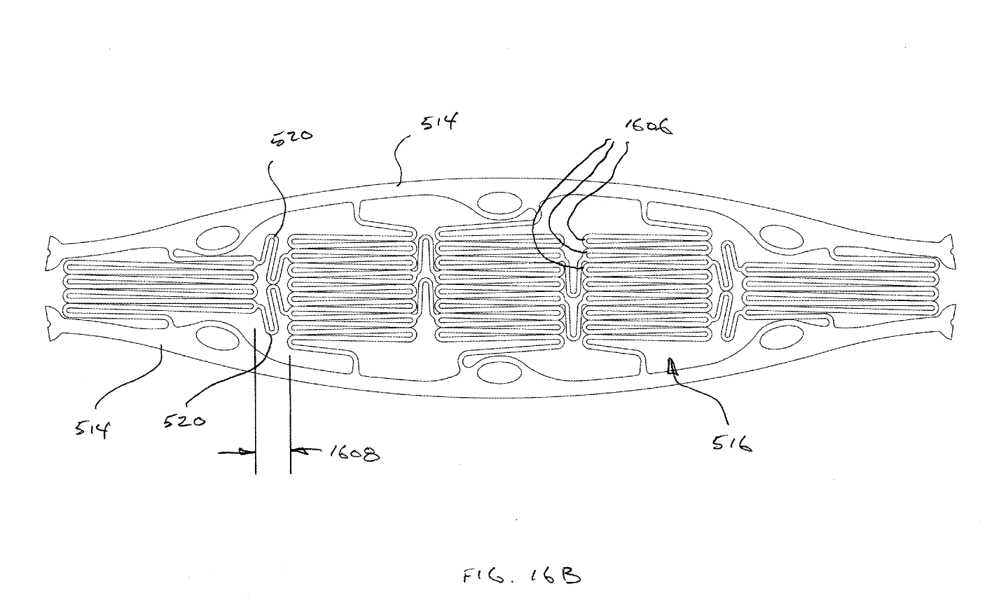

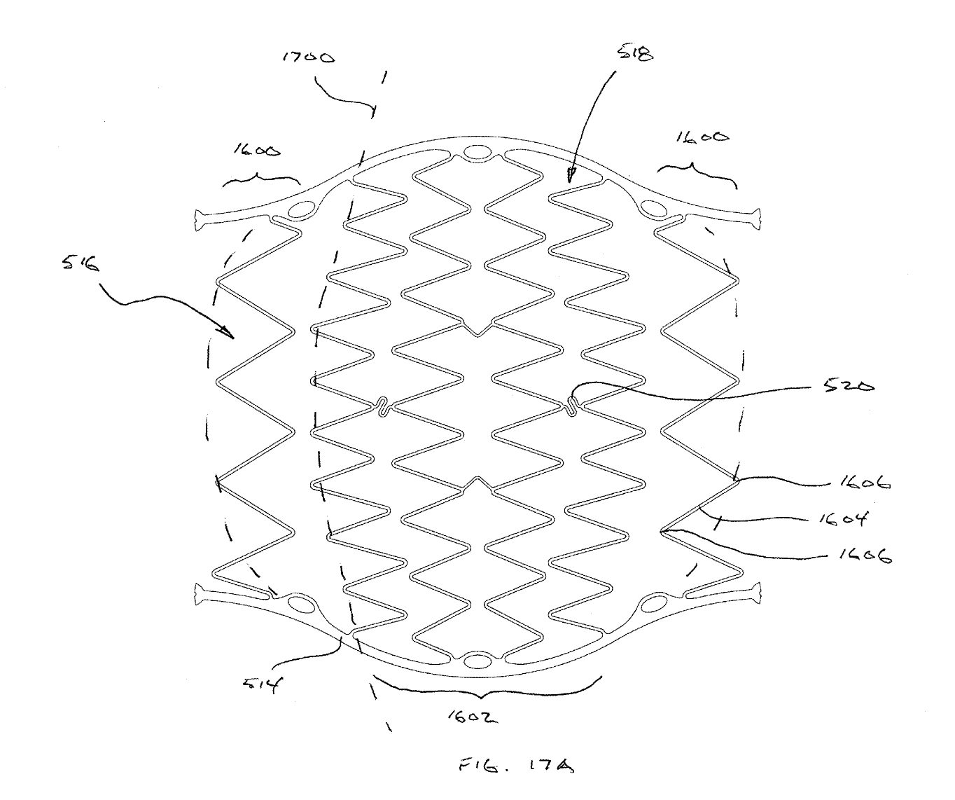

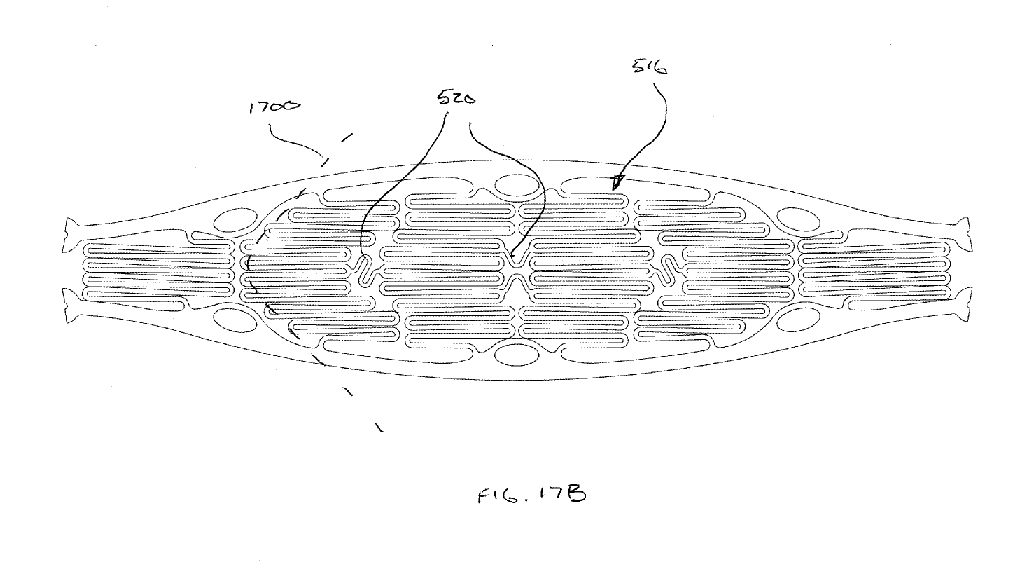

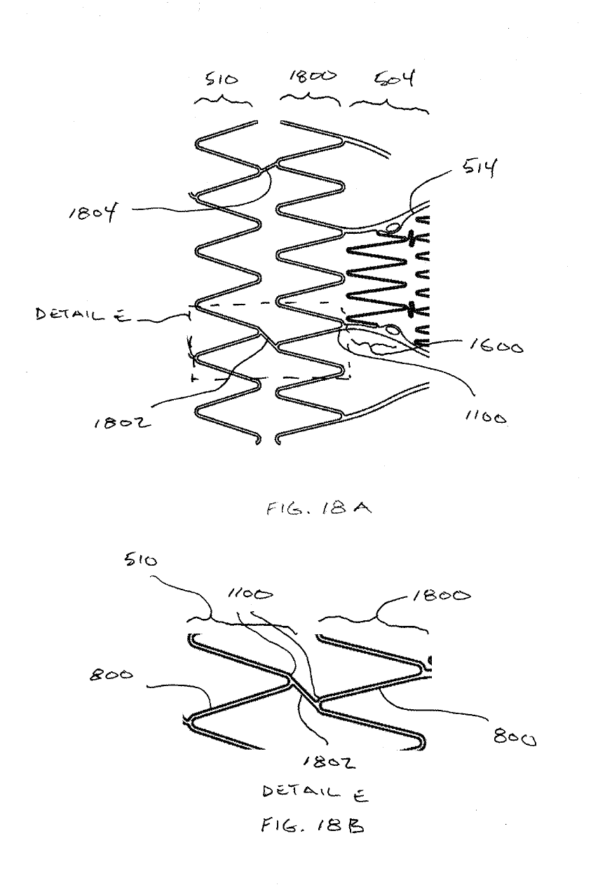

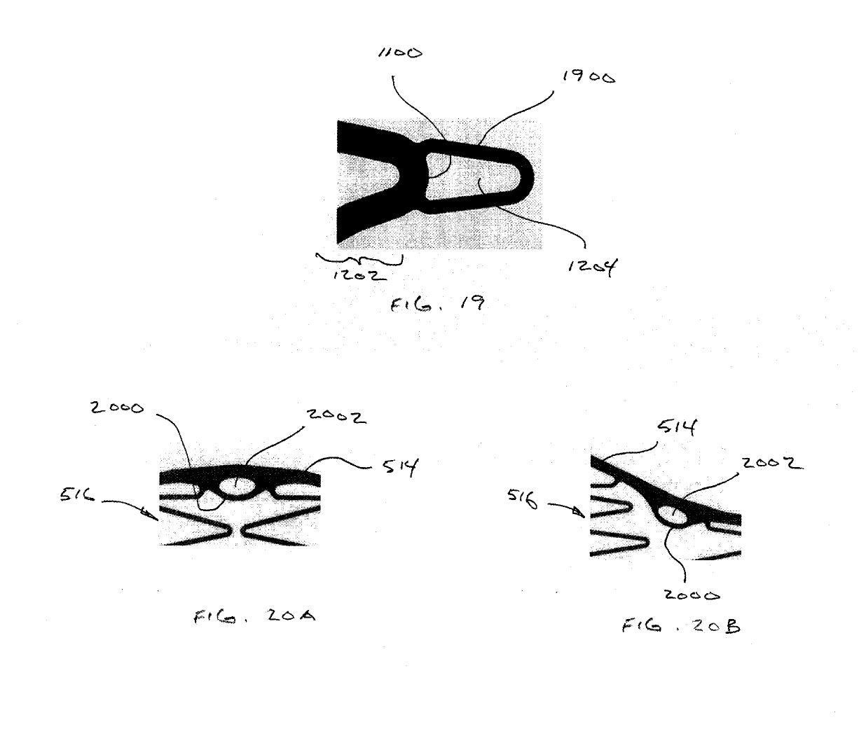

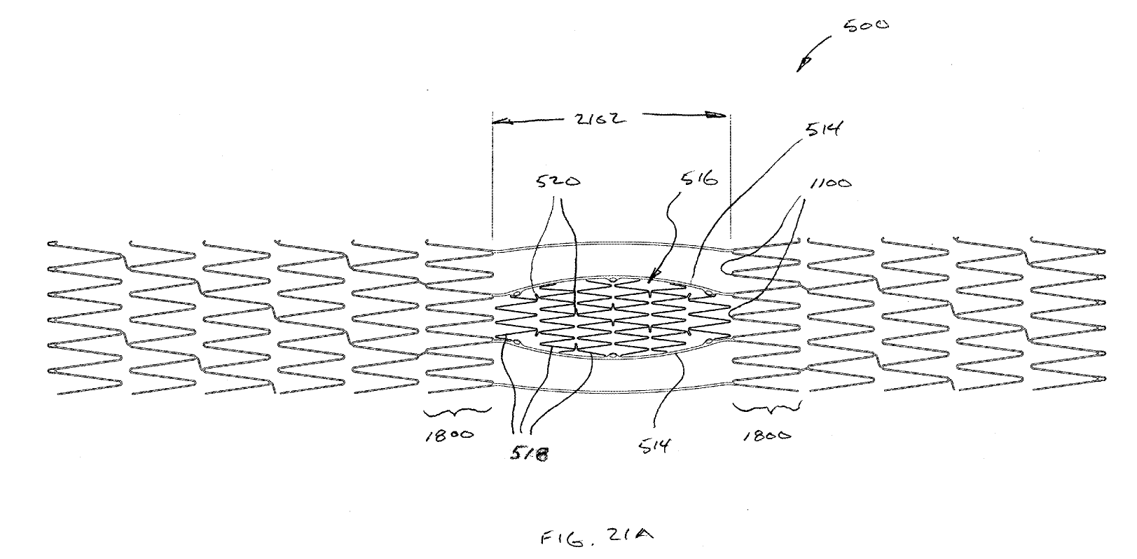

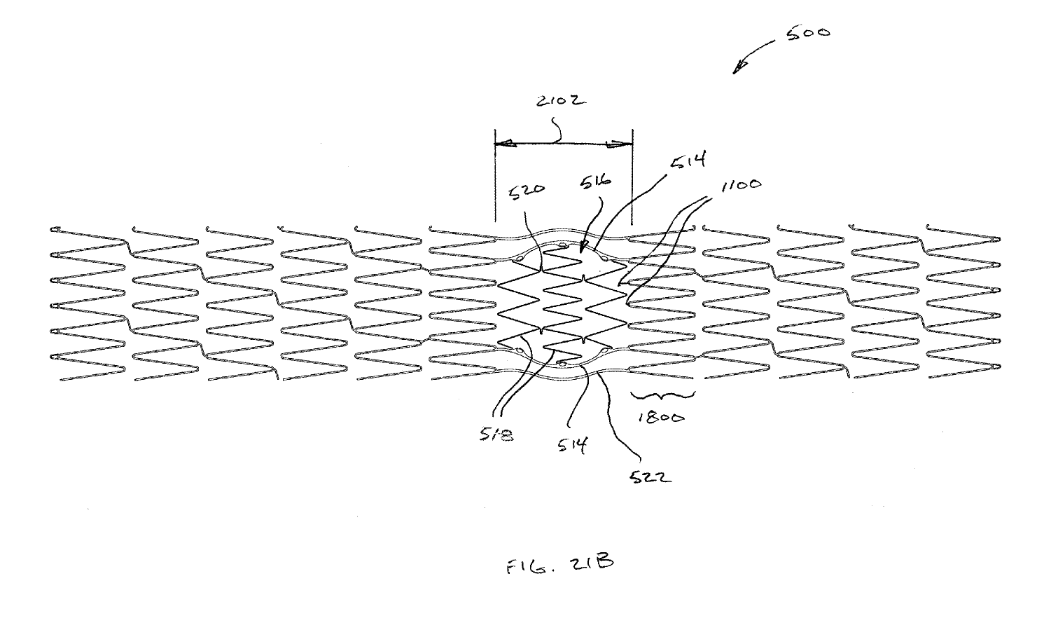

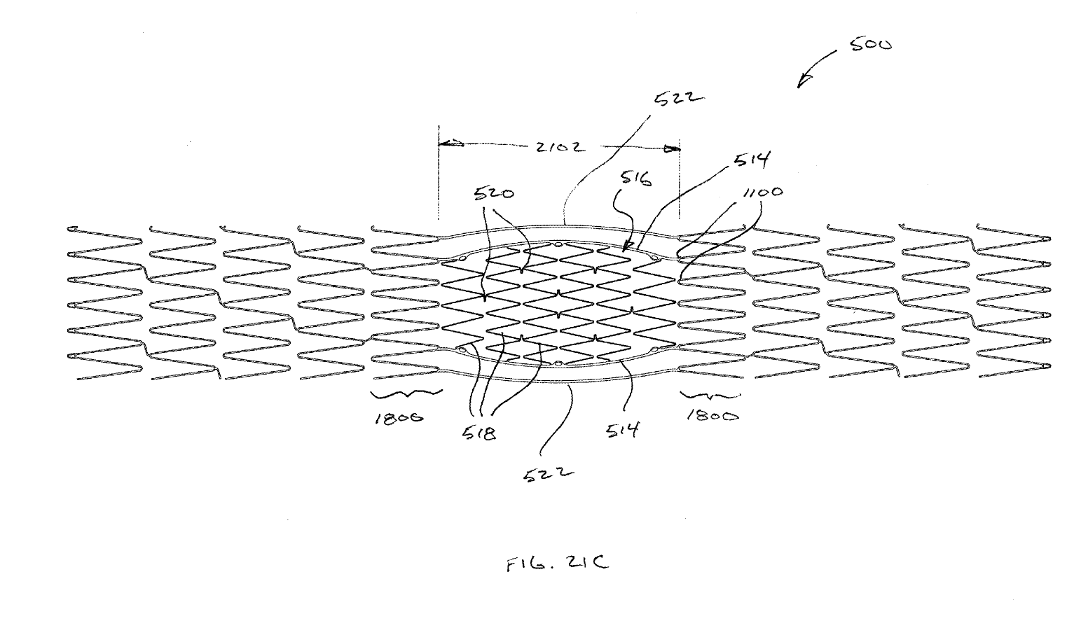

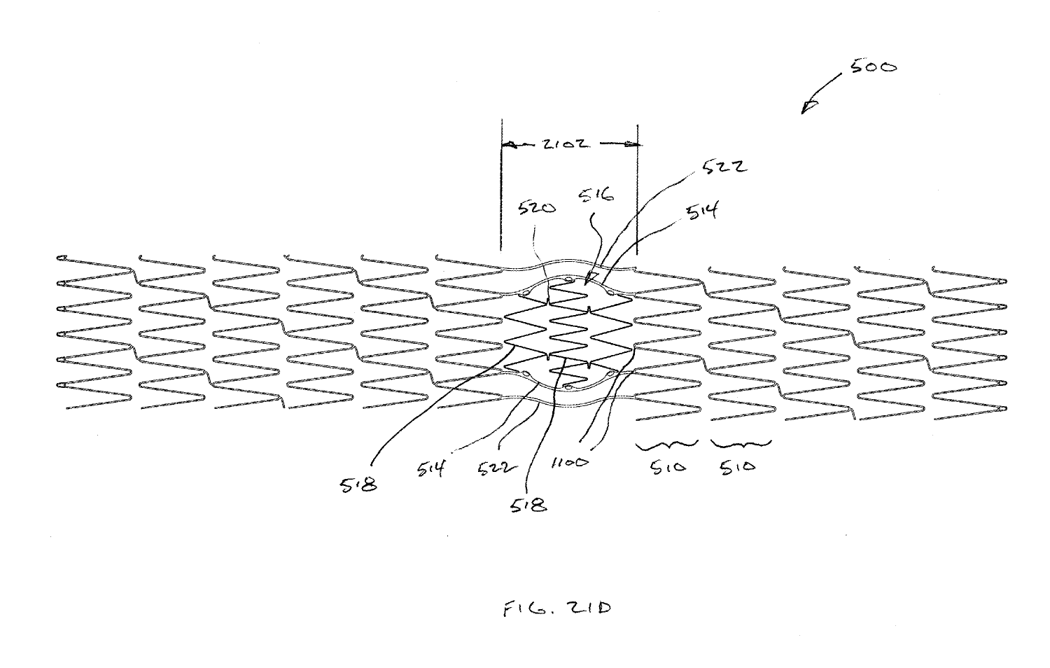

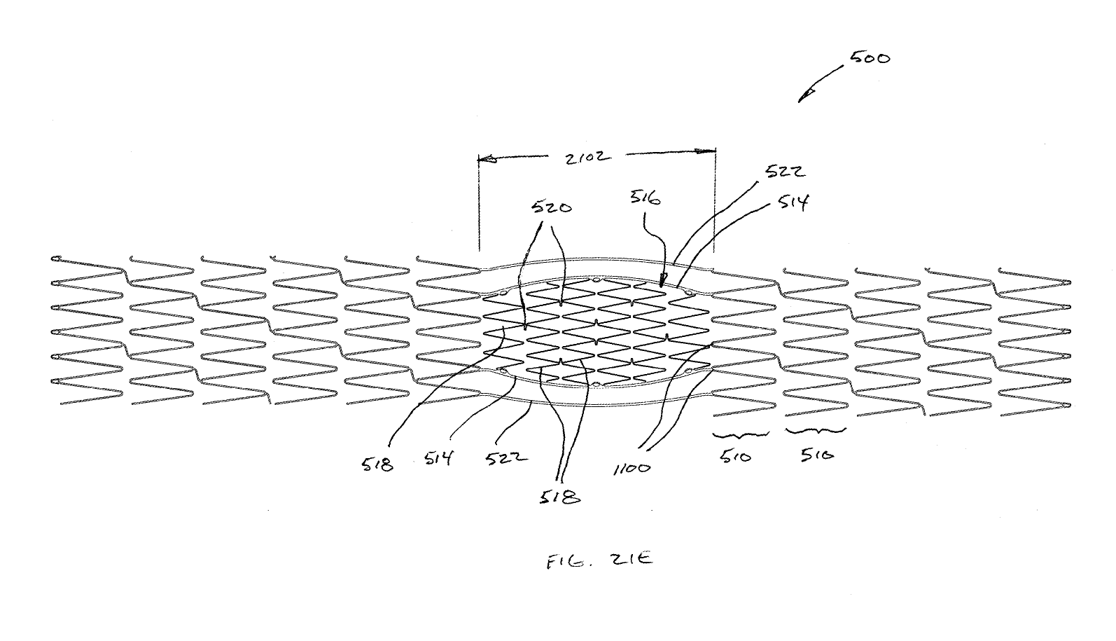

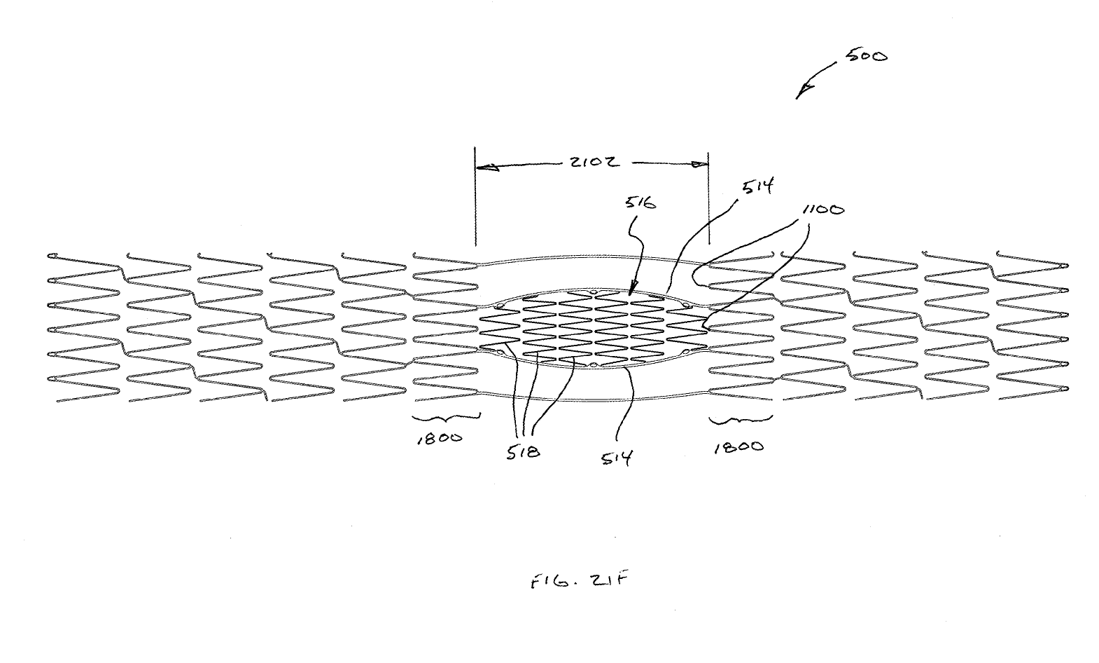

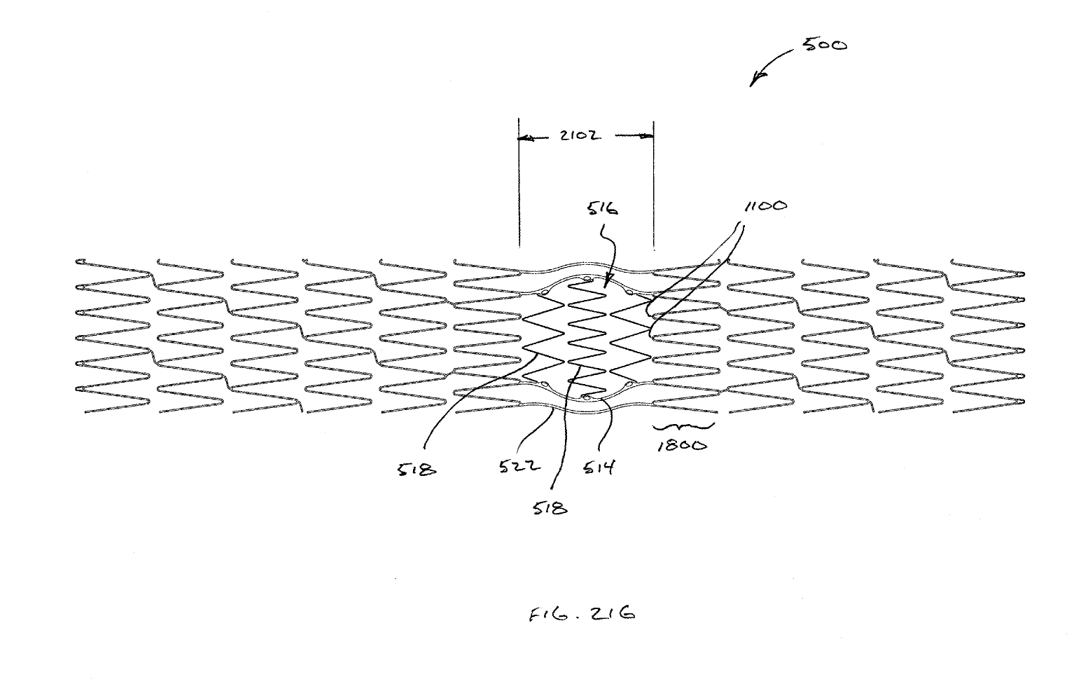

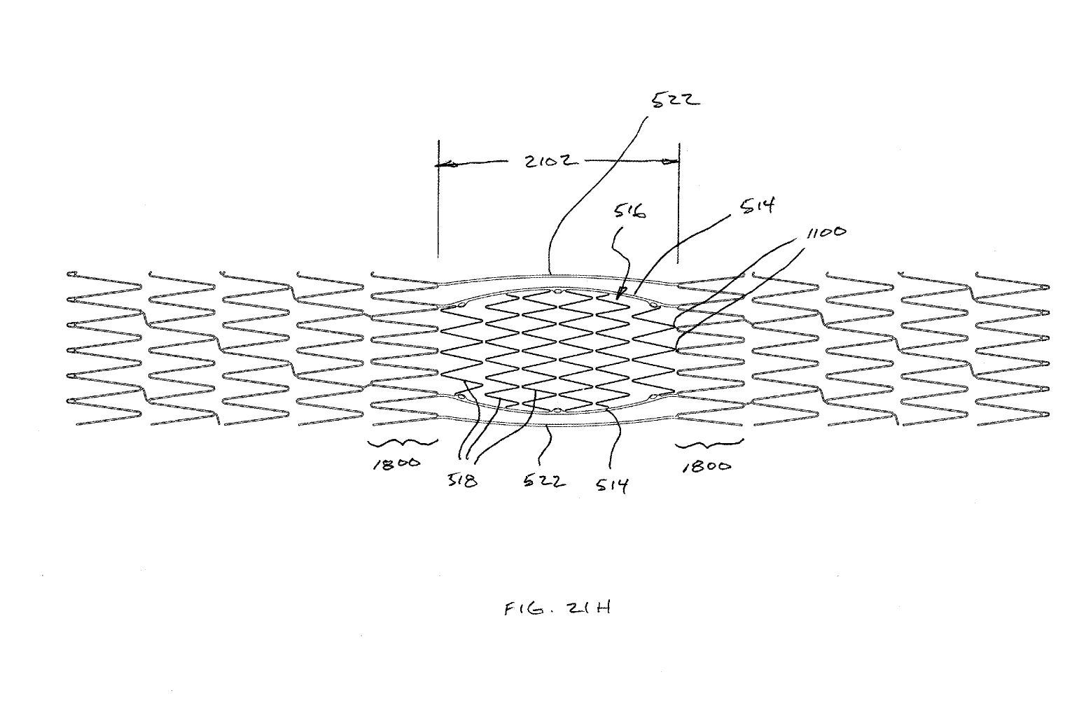

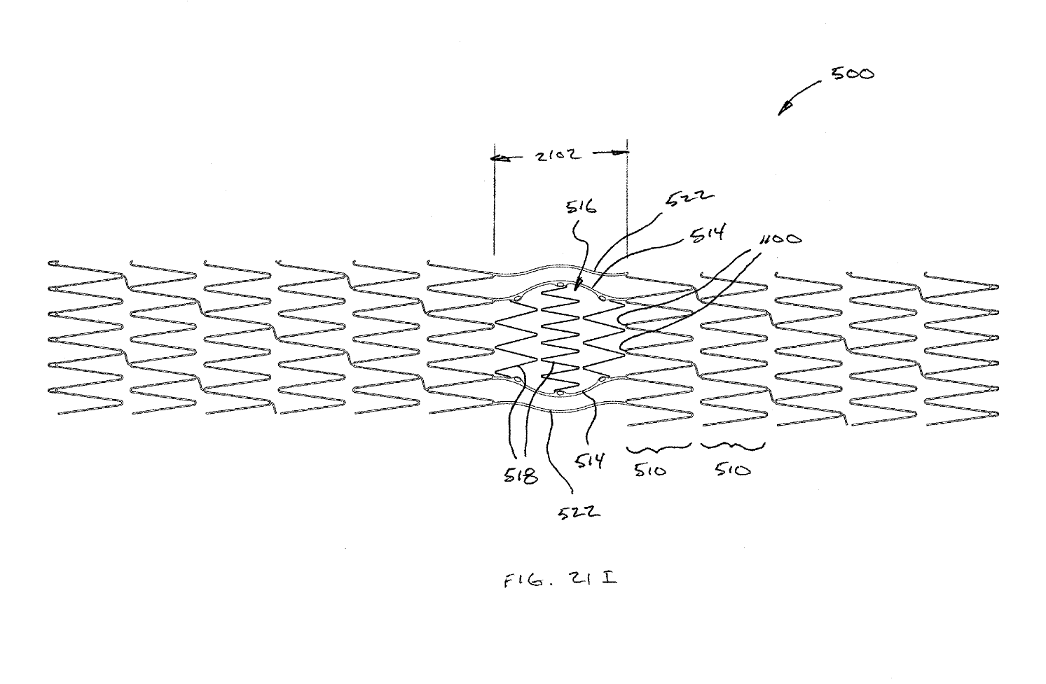

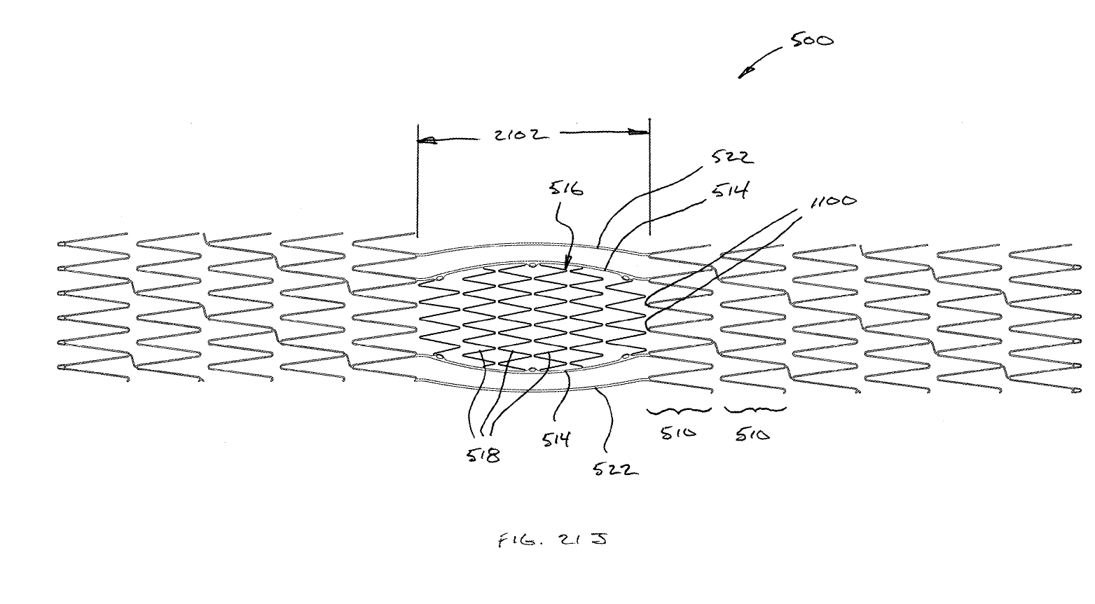

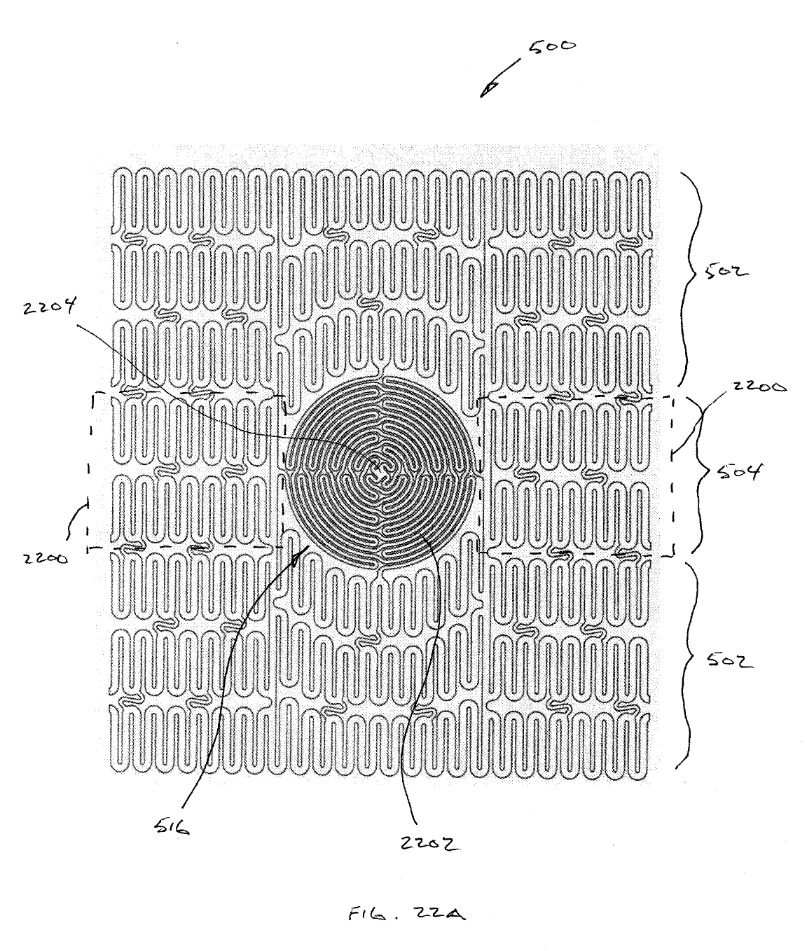

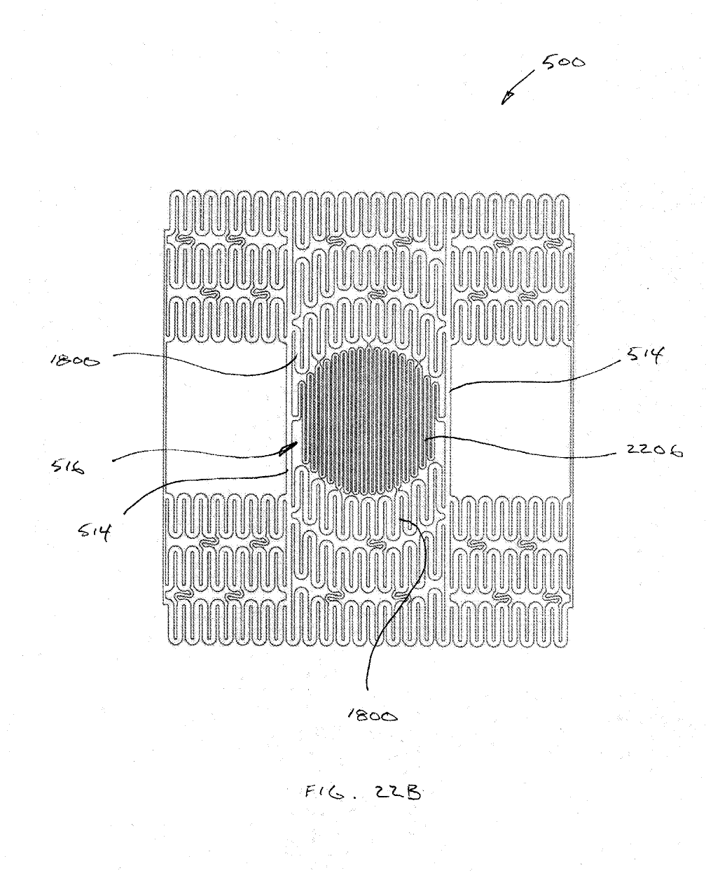

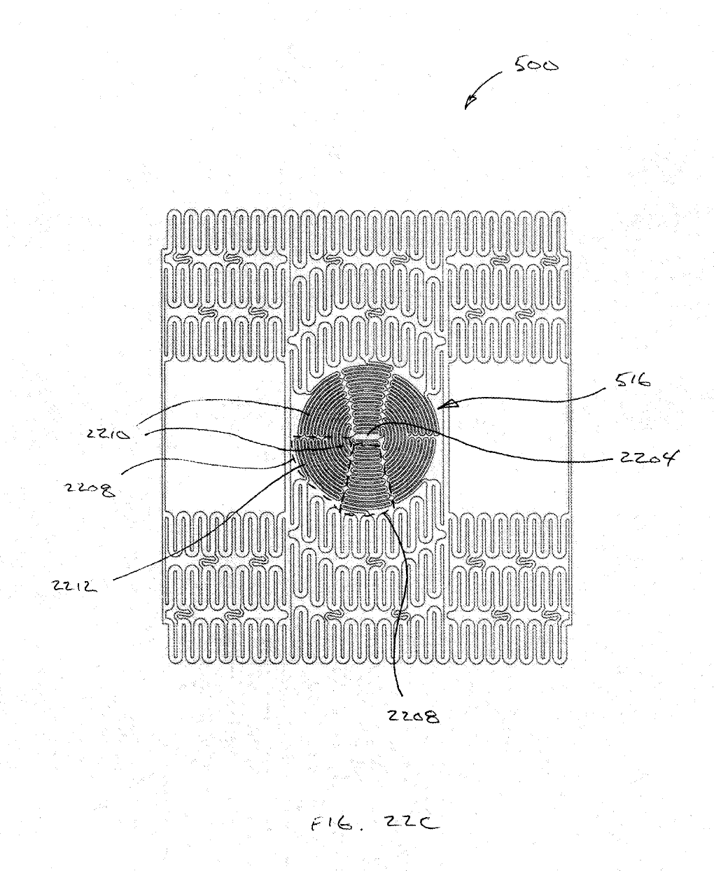

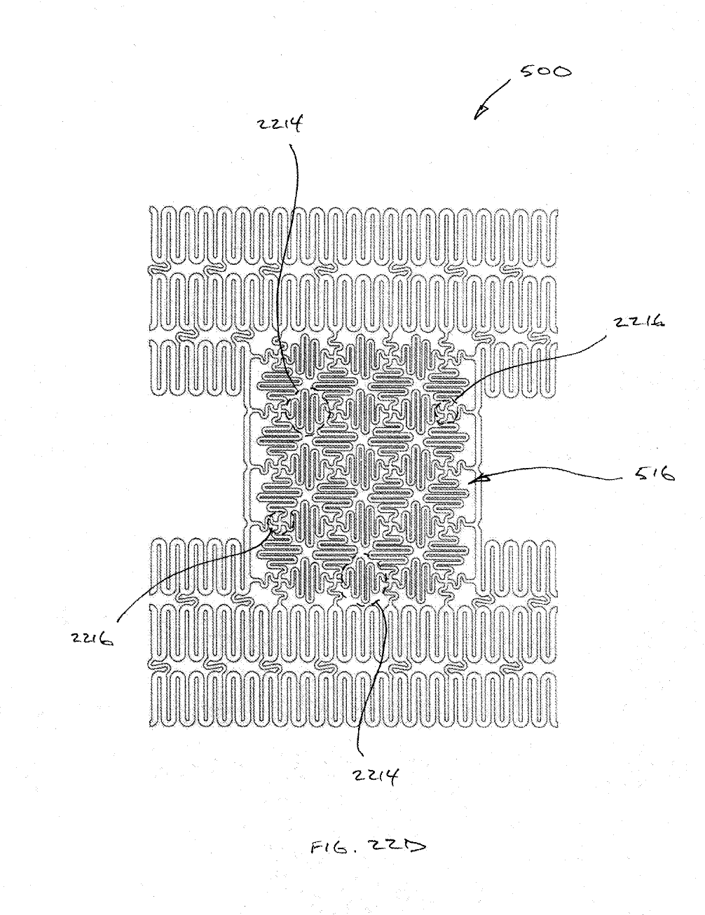

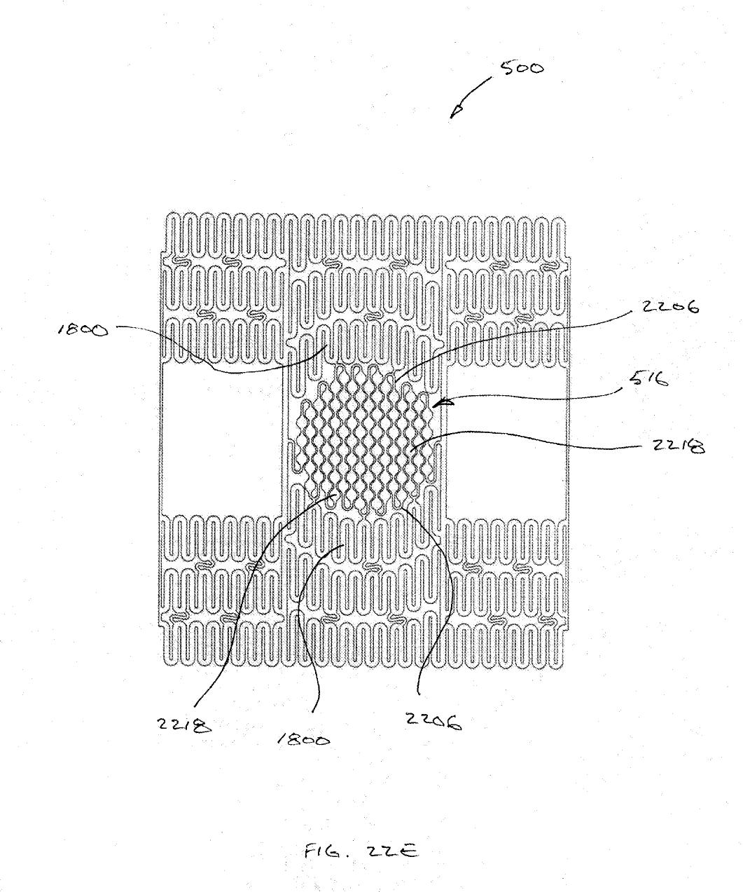

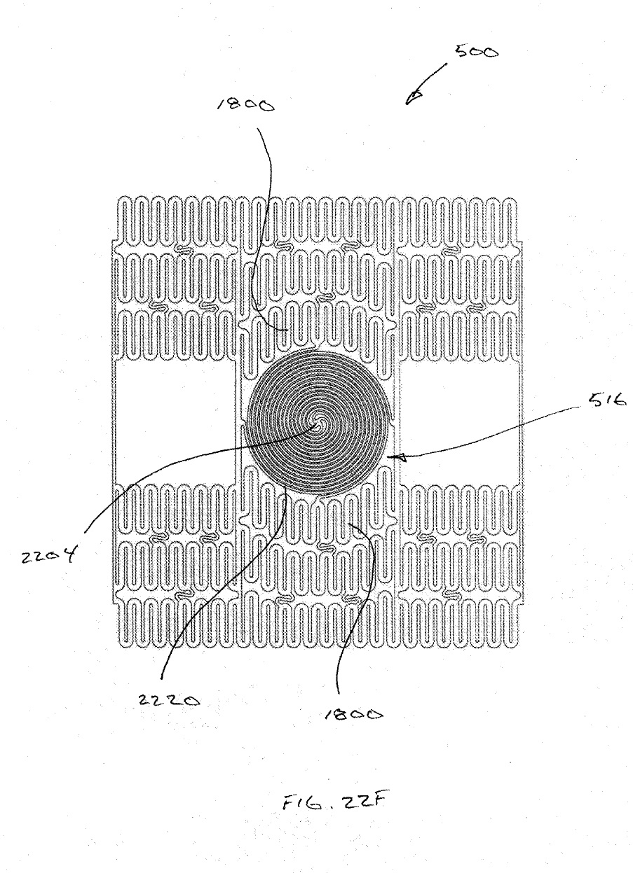

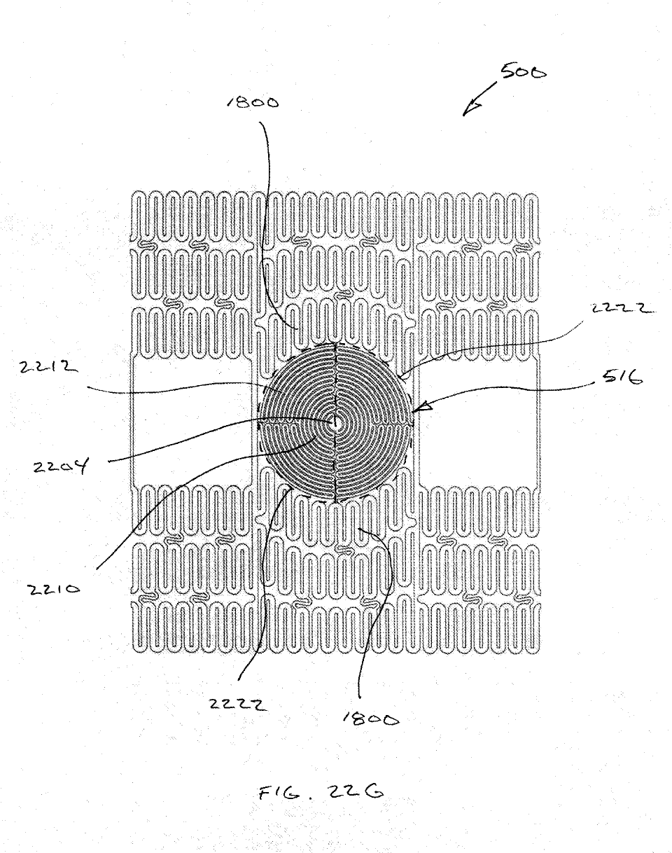

### DETAILED DESCRIPTION OF THE INVENTION

While some embodiments of the present invention are described with specific regard to neurovascular applications, the embodiments of the invention are not so limited and certain embodiments may also be applicable to the treatment of aneurysms in other body vessels. For example, embodiments of the invention may be used to treat aneurysms distal to the origin of the renal arteries, thoracic aortic aneurysms, popliteal vessel aneurysms, or any other body vessel locations.

In various embodiments, description is made with reference to the figures. However, certain embodiments may be practiced without one or more of these specific details, or in combination with other known methods and configurations. In the following description, numerous specific details are set forth, such as specific configurations, dimensions, and processes, in order to provide a thorough understanding of the present invention. In other instances, well-known processes and manufacturing techniques have not been described in particular detail in order to not unnecessarily obscure the present invention. Reference throughout this specification to “one embodiment,” “an embodiment”, or the like, means that a particular feature, structure, configuration, or characteristic described in connection with the embodiment is included in at least one embodiment of the invention. Thus, the appearances of the phrase “one embodiment,” “an embodiment”, or the like, in various places throughout this specification are not necessarily referring to the same embodiment of the invention. Furthermore, the particular features, structures, configurations, or characteristics may be combined in any suitable manner in one or more embodiments.

As described throughout this disclosure, the terms “substantially” and “generally” are used to indicate that the description approximates an actual configuration of an embodiment of the invention. For example, in a description that refers to an implant section as being “substantially cylindrical”, it is to be appreciated that the section may not extend fully around the circumference of the implant, but that one skilled in the art would recognize the section as extending almost entirely around the circumference in a cylindrical manner.

In an aspect, embodiments of the invention describe implants and methods for treating aneurysms. In an embodiment, a single implant with a modular design is provided to scaffold a parent vessel, form a thrombus within an aneurysm, and retain the thrombus, thereby depressurizing the aneurysm and preventing rupture and hemorrhage. The implant may include a base section to expand against a parent vessel distal and/or proximal to an aneurysm. The base section may both scaffold the parent vessel and anchor the implant within the parent vessel, providing immediate flow restoration. In an embodiment, the implant further includes an aneurysm section extending from the base section and including an aneurysm pattern to expand toward an aneurysm gate. The aneurysm pattern may include a plurality of aneurysm arcs sized and configured to promote clotting over the aneurysm pattern. For example, the aneurysm arcs may be narrower and more densely packed than base section struts to promote clotting over the aneurysm pattern after expansion. As clots form over the surface of the aneurysm pattern, flow into an aneurysm may be gradually reduced and a thrombus may also be formed and retained within an aneurysm sac. Once the aneurysm pattern is covered by clotting and/or the aneurysm sac is filled with clotted blood, the aneurysm becomes depressurized and separated from the parent vessel. Depressurization of the aneurysm may reduce the risk of rupture or hemorrhage of the aneurysm.

In another aspect, the implant with a base section and aneurysm section includes an aneurysm pattern that expands into the aneurysm sac. The aneurysm pattern may expand into the aneurysm sac under self-expansion, or it may be plastically deformed into the aneurysm sac under the expansion force of a secondary device, e.g., a dilatation balloon. Protrusion into the aneurysm may result in blood clotting on the aneurysm pattern independently from clotting on the base section. A protruding aneurysm pattern may also result in formation of eddy current laminar flow, i.e., swirling low speed flow, in blood as the blood passes through the aneurysm pattern, which may further accelerate and promote blood clotting on the aneurysm pattern. A protruding aneurysm pattern may also promote clotting within any gaps between the aneurysm pattern and the aneurysm wall. Thus, a protruding aneurysm pattern may result in clotting on aneurysm pattern independently from a base section. As a result, the administration of blood thinners may not impede clotting of the aneurysm pattern as much as it impedes clotting of the base section. This may result in faster clotting in the aneurysm and more rapid depressurization of the aneurysm sac even under the influence of blood thinners, while simultaneously preventing symptomatic intracranial hemorrhage.

In an aspect, the implant may include an unexpanded and an expanded state, and while the base section may represent a cylindrical contour in both states, the aneurysm pattern may transition from a substantially cylindrical configuration in the unexpanded state to a substantially non-cylindrical configuration in the expanded state. As a result, while the base section expands uniformly against the parent vessel in an embodiment, the aneurysm section may expand non-uniformly, such that the aneurysm pattern is biased toward the aneurysm gate. In other words, the aneurysm pattern may expand less in a circumferential direction than the base section. Accordingly, the aneurysm gate may be scaffolded by the aneurysm pattern to a greater degree than the parent vessel wall is scaffolded by the base section. This design allows for an implant to be formed with an unexpanded profile that can be tracked into tiny vessels while still achieving dense coverage of an aneurysm gate that promotes separation and depressurization of an aneurysm sac.

Referring to FIG. 5, a perspective view of a vascular implant in an unexpanded state is shown in accordance with an embodiment of the invention. In an embodiment, a vascular implant 500 includes a modular design, having a base section 502 and an aneurysm section 504. In an unexpanded state, base section 502 and aneurysm section 504 may be configured in a generally cylindrical form, having an outer surface that wraps around a longitudinal axis in a generally circumferential contour. Aneurysm section 504 may extend longitudinally from base section 502. More particularly, base section 502 may be sub-divided into a distal subsection 506 and a proximal subsection 508, with aneurysm section 504 extending between the base subsections, as shown in FIG. 5. However, in an alternative embodiment, base section 502 may be undivided and aneurysm section 504 may extend from an end of base section 502 without being sandwiched by a second base subsection.

Base section 502 and aneurysm section 504 may include different patterns. More particularly, both base section 502 and aneurysm section 504 may be configured to transition from an unexpanded state to an expanded state, but in an embodiment, the expandable pattern and elements of each are suited to the purpose of each section. For example, whereas base section 502 may be configured to expand and provide substantially uniform circumferential scaffolding to a parent vessel, aneurysm section 504 may be configured to expand and provide preferential scaffolding of an aneurysm gate in the parent vessel. Thus, the stent-like pattern, i.e., the expandable pattern, of each section may vary in a modular fashion.

In an embodiment, base section 502 includes a plurality of base rings 510 interconnected by one or more base ring connectors 512. Base ring connectors 512 may stabilize base rings 510 and provide column strength to implant 500 during and after expansion. Base rings 510 and base ring connectors 512 may include numerous design features toward these ends, as described further below.

In an embodiment, aneurysm section 504 includes a plurality of aneurysm section holders 514 that extend longitudinally away from and/or between base section 502. Aneurysm section holders 514 may support aneurysm pattern 516 and provide column strength and stability to implant 500. More specifically, aneurysm pattern 516 may include a plurality of aneurysm arcs 518 that are supported by, and extend radially between, aneurysm section holders 514. In various embodiments, aneurysm pattern 516 may include a variety of aneurysm arc 518 patterns, and in one or more variations, aneurysm arcs 518 may be interconnected by one or more aneurysm pattern connectors 520. Aneurysm arcs 518 in aneurysm pattern may be discontinuous, i.e., aneurysm arcs 518 may not be rings like base rings 510, but may instead be a segment of a circle with ends that do not touch. Some of these embodiments are described further below.

In addition to aneurysm section holders 514 and aneurysm pattern 516, aneurysm section 504 may optionally include one or more aneurysm connectors 522. The number and position of aneurysm connectors 522 are not fixed, but rather are optional, and therefore may be adjusted. In an embodiment, aneurysm connectors 522 may extend away and/or between base section 502 in a manner similar to aneurysm section holders 514. However, in an embodiment, aneurysm connectors 522 are located opposite of aneurysm section holders 514 from aneurysm pattern 516, and thus, do not interconnect with aneurysm arcs 518. More specifically, aneurysm connectors 522 may be configured to scaffold a portion of a parent vessel 202 that is longitudinally aligned, but circumferentially offset from, an aneurysm gate 204. Accordingly, aneurysm connectors 522 may provide radial support opposing any loading of aneurysm pattern 516 by aneurysm gate 204. In addition to providing radial support, aneurysm connectors 522 may also provide column strength to implant 500 in a longitudinal direction, similar to aneurysm section holders 514. In an embodiment, aneurysm connectors 522 and aneurysm section holders 514 may be evenly distributed around a circumference of aneurysm section 504.

Still referring to FIG. 5, the modular implant is shown in an unexpanded state. In this state, the implant 500 may be ready for delivery into a patient for deployment at an aneurysm site. That is, an unexpanded state may refer to a state in which the modular implant is configured to be delivered, which may be an as-cut or a crimped state, in various embodiments. In an embodiment, both base section 502 and aneurysm section 504 are configured in a generally cylindrical form in the unexpanded state. More specifically, aneurysm pattern 516 may be substantially cylindrical in the unexpanded state. As referred to here, substantially cylindrical means that although the aneurysm pattern 516 may not circumscribe the entire circumference of implant 500, the aneurysm pattern 516 does wrap substantially around the longitudinal axis. In an embodiment, one or more aneurysm arcs 518, or a geometric cord extended between aneurysm section holders 514, traverse an angle greater than about 180 degrees in the unexpanded state. For example, the traversed angle may be between about 275 to 360 degrees in the unexpanded state. More particularly, in an embodiment, a portion of aneurysm pattern 516 sweeps across an angle of about 320 degrees between the aneurysm section holders 514 in the unexpanded state.

Referring to FIG. 6A, a perspective view of a vascular implant in an expanded state is shown in accordance with an embodiment of the invention. In an embodiment, both base section 502 and aneurysm section 504 expand toward a generally cylindrical configuration in an expanded state. More particularly, a profile of implant 500 may be generally cylindrical, just as a profile of a parent vessel extending across an aneurysm site is generally cylindrical, notwithstanding an aneurysm sac portion of the parent vessel. However, despite aneurysm section 504 having a generally cylindrical profile, aneurysm pattern 516 may include a substantially non-cylindrical profile in the expanded state. In an embodiment, one or more aneurysm arcs 518, or a geometric cord extending between aneurysm section holders 514, may traverse an angle between about 45 to 300 degrees in the expanded state. For example, the traversed angle may be in a range of about 60 to 275 degrees in the expanded state. More particularly, in an embodiment, a portion of aneurysm pattern 516 sweeps across an angle of about 150 degrees in the expanded state. Geometrically, aneurysm pattern 516 may be described as have a contour of a cylindrical segment, meaning that the profile wraps around a portion of a cylinder dissected by an intervening plane. In an embodiment, the plane may be curvilinear in a generally longitudinal direction and may be offset radially from the longitudinal axis of implant 500. Thus, aneurysm pattern 516 may be considered to be a longitudinal cylindrical segment.

As aneurysm pattern 516 expands toward an expanded state, e.g., toward a longitudinal cylindrical segment shape, aneurysm pattern 516 unfurls to cover less of aneurysm section 504 in a circumferential direction than aneurysm pattern 516 covers of aneurysm section 504 in the unexpanded state. Furthermore, since aneurysm section holders 514 extend longitudinally from base section, an uncovered area 602 of aneurysm section 504 opposite of aneurysm section holders 514 from aneurysm pattern 516 also grows. In an embodiment, uncovered area 602 does not include struts extending in a circumferential direction. Thus, with the exception of perhaps aneurysm connectors 522, uncovered area 602 does not provide radial scaffolding to a vessel. Accordingly, in the unexpanded state, aneurysm pattern 516 may wrap around aneurysm section 504 to fill in uncovered area 602 and, in the expanded state, aneurysm pattern 516 may be directed toward an aneurysm gate while uncovered area 602 may be around an unscaffolded portion of a vessel without the aneurysm gate.

Referring to FIG. 6B, a perspective view of a vascular implant in an expanded state is shown in accordance with an embodiment of the invention. In an embodiment, base section 502 expands toward a generally cylindrical shape while aneurysm section 504 expands toward a non-cylindrical, e.g., bulbous, shape. Base section 502 of FIG. 6B may be configured similar to base section 502 of FIG. 6A. Thus, base section 502 of FIG. 6B may be expanded to radially scaffold a parent vessel segment on either side of an aneurysm. However, aneurysm section 504 of FIG. 6B, and particularly aneurysm pattern 516, may differ from the configuration of aneurysm section 504 of FIG. 6A. In an embodiment, rather than expanding toward a cylindrical segment contour, aneurysm pattern 516 may extend toward a bulbous contour. The bulbous contour may, for example, approximate the bulge of an aneurysm sac away from a parent vessel. Thus, when expanded at an aneurysm site, the aneurysm pattern 516 may protrude into an aneurysm sac even as aneurysm section holders 514 and aneurysm connectors 522 longitudinally scaffold the parent vessel.

In the case shown in FIG. 6B, aneurysm arcs 518 may be discontinuous and extend only around a portion of the circumference of implant 500. More specifically, aneurysm arcs 518 may be circular segments, as opposed to rings, and thus they may terminate at aneurysm section holders 514. Furthermore, uncovered area 602 may be larger in the expanded state, between aneurysm section holders 514 opposite from aneurysm pattern 516, as compared to in the unexpanded state.

Referring to FIG. 7, a side view of a vascular implant in an unexpanded state is shown in accordance with an embodiment of the invention. This configuration corresponds to that of FIG. 5 and shows that implant 500 profile is generally cylindrical about a longitudinal axis. One or more aneurysm arcs 518 extend radially between aneurysm section holder 514 around the implant 500 to form aneurysm pattern 516. In an embodiment, aneurysm arcs 518 are circular segments, and thus, are discontinuous with ends that do not touch each other, in contrast to continuous base rings 510.

Referring to FIGS. 8A-8C, cross-sectional views taken about line A-A of FIG. 7, of a base section of a vascular implant transitioning from an unexpanded state to an expanded state are shown in accordance with an embodiment of the invention. FIG. 8A illustrates base ring 510 in an unexpanded configuration. For example, base ring 510 may be crimped for intravascular delivery into cerebral vessels of a patient. The cross-section illustrates a series of base struts 800 arranged in a ring pattern to form base ring 510. Base ring 510 profile is generally circular, and thus, base section 502 profile may be generally cylindrical also. FIG. 8B illustrates base ring 510 after it has been partially expanded. The cross-section indicates that base struts 800 expand in a generally uniform manner, consistent with an objective of uniformly scaffolding a vessel wall. FIG. 8C illustrates base ring 510 after it has been fully expanded. The cross-section illustrates that base struts 800 have expanded to a deployed diameter, e.g., into apposition with a parent vessel. Furthermore, the expanded base struts 800 remain uniformly spread around a circumference, causing a profile of base ring 510 to remain in a cylindrical shape.

In an alternative embodiment, an expanded base ring 510 may not include base struts 800 that are as uniformly distributed as those shown in FIG. 8C. For example, in the case of a balloon expandable stent, uneven pressure applied to the stent surface during expansion may cause a region of base ring 510 to include denser strut spacing than a circumferentially opposite region. However, even in such cases, base ring 510 may include a continuous structure that provides radial scaffolding around an entire circumference of a parent vessel. This continuous structure may be contrasted with discontinuous aneurysm arcs 518 that do not extend fully around implant 500 circumference.

Referring to FIGS. 9A-9C, cross-sectional views taken about line B-B of FIG. 7, of an aneurysm section of a vascular implant transitioning from an unexpanded state to an expanded state are shown in accordance with an embodiment of the invention. FIG. 9A illustrates aneurysm section 504 in an unexpanded configuration. As mentioned above, the unexpanded configuration may refer to a state in which the vascular implant is configured for delivery through a patient vasculature, e.g., a crimped, as-cut, or otherwise compact diameter state. In the illustrated embodiment in the unexpanded configuration, aneurysm pattern 516 between aneurysm section holders 514 sweeps through an angle of about 320 degrees. Thus, uncovered area 602 sweeps through an angle of about 40 degrees in the configuration shown. Accordingly, aneurysm pattern 516 may be considered to have a substantially cylindrical contour in the unexpanded state. Aneurysm connectors 522 are located opposite of aneurysm section holders 514 from aneurysm arcs 518 of aneurysm pattern 516, and at least partly fill a gap of uncovered area 602 between aneurysm section holders 514. In other words, aneurysm connectors 522 are located within the space not traversed by aneurysm pattern 516. Accordingly, taken together, aneurysm pattern 516, aneurysm section holders 514, and aneurysm connectors 522 make up aneurysm section 504 have a generally cylindrical profile.

The cross-section shown in FIG. 9A is taken through a medial section of aneurysm pattern 516, and thus, aneurysm pattern 516 including aneurysm struts 900 may include an aneurysm arc 518 that sweeps through a greater angle than an aneurysm arc 518 nearer to base section 502. The reason for the greater angle in the medial section is that aneurysm section holders 514 may bow outward circumferentially from their attachment to base section 502, and thus describe a larger angle near the apex of that bow than at the beginning of the bow. In an embodiment, aneurysm section holders 514 may be separated by only one or a few base struts 800 at the longitudinal location where aneurysm section 504 meets base section 502. For example, aneurysm section holders 514 may be separated by one to three base joints where aneurysm section 504 meets base section 502. Thus, an aneurysm arc 518 or a geometric cord between aneurysm section holders 514 near that location may sweep through an angle in an unexpanded state of between about 100 to 200 degrees. For example, the angle between aneurysm section holders 514 near a location at which aneurysm section 504 meets base section 502 may be about 150 degrees in an unexpanded state when two base joints separate aneurysm section holders 514. Despite the angular difference between aneurysm section holders 514 at medial and end locations, the overall profile of aneurysm pattern 516 may be substantially cylindrical in the unexpanded state, and as described above, at least one aneurysm arc 518 extending radially between aneurysm section holders 514 may sweep through an angle that is substantially circumferential, e.g., greater than about 180 degrees, and more particularly, greater than about 275 degrees.

FIG. 9B illustrates aneurysm section 504 in a partially expanded state. As aneurysm section 504 expands from the unexpanded state, aneurysm pattern 516 begins to bias toward a side of aneurysm section 504. More particularly, aneurysm pattern 516 expands less in a circumferential direction than a corresponding segment of base ring 510 illustrated in FIG. 8B, and as a result, the sweep angle of aneurysm arc 518 between aneurysm section holders 514 decreases. In a partially expanded state, the sweep angle may decrease to less than 275 degrees, e.g., to about 200 degrees. Thus, the aneurysm pattern 516 may begin to transition toward an ultimate configuration that may no longer be considered to be substantially cylindrical. During expansion, aneurysm connectors 522 may also expand in a circumferential direction and a gap 902 between aneurysm connectors 522 may widen. Together, although aneurysm pattern 516 may no longer be substantially cylindrical, aneurysm section 504 may still have a profile that is generally cylindrical.

FIG. 9C illustrates aneurysm section 504 in a fully expanded state. In the fully expanded state, aneurysm section 504 may appose a region of a parent vessel and may be collinear with the parent vessel along another region. More particularly, a region of aneurysm section 504 including a portion of aneurysm pattern 516, may scaffold across an aneurysm gate. Therefore, the fully expanded aneurysm section 504 of FIG. 9C may be generally cylindrical since a circle may be circumscribed through the aneurysm struts 900, aneurysm section holders 514, and aneurysm connectors 522. Nonetheless, aneurysm pattern 516 may be considered to be substantially non-cylindrical, since it sweeps through an angle of less than about 275 degrees, e.g., through an angle of about 150 degrees, in an embodiment. Geometrically, the profile of aneurysm pattern 516 may be referred to as a longitudinal cylindrical segment, since the contour of aneurysm pattern 516 resembles that of a cylinder dissected through aneurysm section holders 514 by a longitudinal plane. In an embodiment, aneurysm pattern 516 does not form a continuous structure around an entire circumference of a parent vessel 202, but rather, scaffolds only a portion of a vessel circumference, e.g., an aneurysm gate.

In an embodiment, aneurysm pattern 516 covers a generally elliptical area, since the distance between aneurysm section holders 514 near the base section may be less than the distance between aneurysm section holders 514 near the medial section. More particularly, in an expanded configuration, an angle between aneurysm section holders 514 near the medial section may be about 150 degrees while the angle between aneurysm section holders 514 near the base section may be about 100 degrees. Thus, the perimeter of aneurysm section 516 may resemble an ellipse that is projected against a cylindrical shape of a patient vessel. By way of example, such an ellipse may include a dimension along a major axis in a range of about 10 to 12 mm or more, while a dimension along a minor axis may be in a range of about 5 to 10 mm. These dimensions are provided by way of example, though, and as described below certain configurations may include dimensions along a major axis in a range of about 5 to 10 mm and therefore be similar to a dimension along the minor axis. Thus, aneurysm section 516 may resemble a projected circle rather than a projected ellipse.

Referring to FIG. 10, a cross-sectional view taken about line B-B of FIG. 7, of an aneurysm section of a vascular implant in an expanded state is shown in accordance with an embodiment of the invention. In an embodiment, aneurysm section 504 does not assume a cylindrical profile at full expansion, but rather, aneurysm section 504 profile may be egg-shaped, elliptical, figure-eight-shaped, etc. More particularly, aneurysm pattern profile 1000 defined by a shape passing through aneurysm struts 900 and aneurysm section holders 514 may not be concentric with an aneurysm connector profile 1002 defined by a shape passing through aneurysm connectors 522 and aneurysm section holders 514. In other words, aneurysm pattern 516 may include a contour that is non-cylindrical and which forms a bulbous shape that bulges away from a parent vessel 202. In alternative embodiments, the bulging profile of aneurysm pattern 516 may have numerous shapes, including a saccular shape or a fusiform shape. That is, aneurysm pattern 516 may be designed to include various patterns, which upon expansion, assume any shape that bulges away from the cylindrical form of base section 502, permitting aneurysm pattern 516 to protrude into an aneurysm from a parent vessel. A non-cylindrical contour may include cylindrical segments, bulges, curved ellipsoid shapes, circular shapes projected onto a curved plane, etc. Essentially, shapes that do not wrap almost entirely around a longitudinal axis fully to form a continuous, or nearly continuous, cylinder may be considered non-cylindrical.

FIG. 11A illustrates a flat pattern of a vascular implant 500 having arcuate aneurysm connectors 522 in accordance with an embodiment of the invention. The flat pattern of implant 500 illustrates a pattern that may be wrapped about a longitudinal axis to result in the tubular implant 500 of FIG. 5. More specifically, the flat pattern may be interpreted by computer-aided manufacturing software to control machine tools, such as laser cutting equipment, that can cut raw tubing to form a cylindrical implant 500. Many of the same elements as previously illustrated are represented in the figure, and in addition, aneurysm section holder 514 and aneurysm connector 522 profiles are more readily apparent. Aneurysm section holders 514 and aneurysm connectors 522 may extend longitudinally between base subsections along a non-linear, e.g., arcuate, path. For example, in an embodiment, aneurysm section holder 514 and/or aneurysm connector 522 may undulate through a single wave while extending from a first base joint 1100 of a base ring 510 in proximal subsection 508 to a second base joint 1100 of a base ring 510 in distal subsection 506.

In an embodiment, aneurysm connector 522 and an adjacent aneurysm section holder 514 have conforming shapes such that their bodies nest with each other when implant 500 is crimped to a smaller diameter. For example, as shown in FIG. 11A both aneurysm section holder 514 and aneurysm connector 522 follow arcuate paths that reach a lateral apex 1102 near a medial aneurysm arc 1103. The conforming arcuate shapes may allow for aneurysm pattern 516 to wrap more fully around cylindrical implant 500 such that medial aneurysm arc 1103 sweeps through a nearly circumferential angle in an unexpanded, e.g., crimped, state.

Aneurysm section holders 514 and aneurysm connectors 522 may be sized and configured to provide adequate radial support to a parent vessel yet be flexible enough to bend and conform to curved vessel walls, such as in the case where an aneurysm is located in a bifurcated or tortuous vessel. For example, aneurysm section holders 514 and aneurysm connectors 522 may include a width, meaning a lateral dimension within the plane of FIG. 11A, of between about 0.002-inch to 0.006-inch. More particularly, the width may be in a range between about 0.003-inch to 0.004-inch. More particularly, in an embodiment, a width of aneurysm section holders 514 and/or aneurysm connectors 522 may be about 0.0036-inch. A thickness of aneurysm section holders 514 and/or aneurysm connectors 522 into the plane of FIG. 11A may be in a range of about 0.001-inch to 0.006-inch. More particularly, the thickness may be in a range between about 0.002-inch to 0.004 inch. For example, a thickness of base struts 800 may be about 0.0024-inch.

In an embodiment, aneurysm section holders 514 and aneurysm connectors 522 may extend away from a base joint 1100 at a location that is laterally offset from the base joint 1100 apex. For example, rather than extending directly from the apex, the holder or connector may extend from a location on base joint 1100 near a point where the base joint 1100 transitions into an adjoining base strut 800. Thus, a profile along one side of base strut 800 may transition smoothly into a profile along a side of aneurysm connector 522 and aneurysm section holders 514. Accordingly, holders or connectors may extend generally in a longitudinal direction, although at the point of union with a base joint 1100, they may extend radially as well.

FIG. 11B illustrates a flat pattern of a vascular implant 500 having arcuate aneurysm connectors 522 in accordance with an embodiment of the invention. In an embodiment, both aneurysm section holders 514 and aneurysm connectors 522 extend along arcuate paths between base subsections 506, 508, but the arcuate paths may curve in different directions toward lateral apex 1102. Thus, rather than conform with each other, the arcuate paths may in effect be mirror images of each other. As a result, in a crimped or unexpanded state, aneurysm pattern 516 may sweep through a smaller angle of cylindrical aneurysm section 504, since the aneurysm section holders 514 will abut with aneurysm connectors 522 that are spread further apart compared to the embodiment of FIG. 11A. Nonetheless, aneurysm pattern 516 may still sweep through an angle of about 275 degrees or more, between a medial region of aneurysm section holders 514 when the implant is in an unexpanded state, and include a contour that may be considered to be substantially cylindrical.

FIG. 11C illustrates a flat pattern of a vascular implant 500 having undulating aneurysm connectors 522 in accordance with an embodiment of the invention. In an embodiment, aneurysm connector 522 undulates through a plurality of waves 1104 to extend between base subsections 506, 508. For example, the undulating connectors may curve along three waves 1104 between base subsections. In an embodiment, a medial wave 1106 of the plurality of waves 1104 includes a radius of curvature that generally conforms with aneurysm section holder 514. However, in an alternative embodiment, medial wave 1106 may follow an arcuate path opposite to that of aneurysm section holder 514, similar to the configuration of FIG. 11B. Undulating aneurysm connectors 522 may allow sufficient flexibility for aneurysm section 504 to be placed in a tortuous segment of a parent vessel. More specifically, the undulations may allow aneurysm connector 522 to elongate or shorten as necessary to conform to a parent vessel curvature. As a result, implant 500 may be used in a variety of tortuous vessels without failing due to increased bending stresses.

FIG. 11D illustrates a flat pattern of a vascular implant 500 having nested aneurysm connectors 522 in accordance with an embodiment of the invention. In an embodiment, aneurysm connector 522 extends longitudinally from base subsections 506, 508, but follows a path that reverses on itself at least once between ends. For example, aneurysm connector 522 may include one or more extending strut 1110 extending longitudinally from a base subsection toward a reversing strut 1112 that extends longitudinally back toward the originating base subsection. Furthermore, a nesting strut 1114 may reverse paths from the reversing strut 1112 and follow a path that generally conforms with the arcuate path of aneurysm section holder 514. Like the undulating aneurysm connectors 522 of FIG. 11C, nesting segments 1108 of aneurysm connectors 522 may provide greater flexibility and conformability than, for example, a completely straight aneurysm connector 522. However, in addition to providing flexibility that allows for conformance with a curved parent vessel, the nesting segment may conform closely with aneurysm section holders 514 without taking up a significant swath of a cylindrical implant 500 circumference. Thus, implant 500 may be crimped to an unexpanded state such that aneurysm pattern 516 sweeps through an approximately circumferential angle, e.g., an angle greater than about 275 degrees. Accordingly, at least a portion of aneurysm pattern 516 may include a contour that is substantially cylindrical in the unexpanded state.

FIG. 11E illustrates a flat pattern of a vascular implant 500 having double-nested aneurysm connectors 522 in accordance with an embodiment of the invention. Aneurysm connector 522 may include a plurality of nesting segments 1108 having reversing struts 1112 that switchback to act like a spring to provide longitudinal flexibility. Furthermore, aneurysm connector 522 may include a nesting strut 1114 that conforms closely with aneurysm section holder 514. As in, FIG. 11D, aneurysm connectors 522 with nesting segments 1108 may fill a space opposite of aneurysm section holders 514 such that aneurysm section 504 and aneurysm pattern 516 of an unexpanded implant 500 assumes a substantially cylindrical profile.

FIG. 12A illustrates a flat pattern of a base section 502 of a vascular implant 500 having straight base struts 800 in accordance with an embodiment of the invention. In an embodiment, base section 502 shown in FIG. 12A represents either distal subsection 506 or proximal subsection 508. More specifically, distal and proximal subsections 506, 508 may have identical designs, or alternatively, they may vary in the number and configuration of base rings 510, base struts 800, etc. For example, as shown with respect to FIG. 11A, proximal subsection 508 may include four base rings 510 while distal subsection 506 may include five base rings 510. In other embodiments, proximal subsection 508 may include five base rings 510 while distal subsection 506 may have six base rings. Additionally, each base ring 510 of a respective base subsection may include similar or different patterns. By varying a number of base rings 510, the overall length of implant 500 may be varied as needed. In an embodiment, the number and length of base rings 510 may be adjusted to result in an implant 500 with an overall length of between about 15 to 60 mm. For example, the overall length of implant 500 may be adjusted to be within a range of about 15 to 40 mm.

In an embodiment, base section 502 includes a plurality of base rings 510 interconnected by one or more base ring connectors 512. For example, in an embodiment, each base ring 510 is interconnected with an adjacent base ring 510 by two base ring connectors 512. Each base ring 510 may include a plurality of base struts 800 interconnected by base joints 1100 at the ends of each base strut 800. Thus, a base strut 800 may be interconnected with a first adjacent base strut in a same base ring 510 by a first base joint 1100, and the base strut 800 may be interconnected with a second adjacent base strut in the same ring by a second base joint 1100. Base joints 1100 may alternatively be referred to as “crowns”, “peaks”, “elbows”, “knees”, etc. A base joint 1100 articulates when it undergoes material strain to allow a strut angle 1200 between adjacent base struts 800 to change. For example, a strut angle 1200 between adjacent base struts 800 may decrease as implant 500 is crimped, allowing base ring 510 to reduce to a smaller cylindrical diameter. Conversely, strut angle 1200 may increase as it is expanded, allowing base ring 510 to increase to a larger cylindrical diameter. In an embodiment, each base ring 510 includes four or more base joints 1100 to allow for sufficient joint expansion to permit implant 500 to expand from an unexpanded diameter of about 0.5 mm (0.019 in) to a deployed diameter of between about 3 to 6 mm. For example, each base ring 510 may include six base joints 1100 that expand until implant 500 reaches a fully deployed diameter of about 4.25 mm. These diameter ranges are examples and may be adjusted within the scope of this invention according to the description provided.

In an embodiment, base struts 800 may be sized to flex and conform to a parent vessel wall, yet provide radial support to the parent vessel. For example, in an embodiment, a width of base struts 800 within the plane of FIG. 12A is in a range between about 0.01-inch to 0.04-inch. More particularly, a width of base struts 800 may be in a range of about 0.012-inch to 0.036-inch. For example, base strut 800 width may be about 0.020-inch. A thickness of base struts 800 into the plane of FIG. 12A may be in a range of about 0.002-inch to 0.004-inch. For example, a thickness of base struts 800 may be about 0.0024-inch.

In an embodiment, base struts 800 may extend straightly between pairs of base joints 1100. In other words, base struts 800 may follow a linear sawtooth pattern around base ring 510. By following a straight path, base struts 800 may provide column strength in an unexpanded state. Furthermore, as implant 500 transitions from the unexpanded to an expanded configuration, straight base struts 800 may provide a ring structure that radially supports a parent vessel.

Referring to FIG. 12B, a detail view taken from Detail B of FIG. 12A illustrates a base ring connector region of a base section of a vascular implant in accordance with an embodiment of the invention. Base joints 1100 of base rings 510 may be configured to withstand crimping and expansion without material failure. In an embodiment, resistance to failure during a change in diameter may be facilitated in part by properly sizing a base joint inner diameter 1206 to withstand material strain in base joint 1100. For example, in an embodiment, base joint inner diameter 1206 may be in a range of about 0.003-inch to 0.006-inch. More particularly, base joint inner diameter 1206 may be about 0.005-inch.

Still referring to FIG. 12B, base ring connector adjoining adjacent base rings may include a curvilinear shape extending from a first base joint 1210 to a second base joint 1212. The curvilinear shape may for example extend in a longitudinal direction from the first base joint 1210 and then curve in a radial direction before curving again in a longitudinal direction to connect with the second base joint 1212. In an embodiment, the curvilinear shape of base ring connector 512 creates an offset between the first and second base joints 1210, 1212. More particularly, the base joints 1210, 1212 may be offset in a longitudinal and/or circumferential direction. As a result, base ring connector 512 may act like a flexing hinge between the base joints 1210, 1212 to allow the base joints 1100 to move relative to each other.

Referring to FIGS. 13A-13B, flat pattern views illustrating alternative embodiments of base ring connector regions of a base section of a vascular implant pattern are shown in accordance with an embodiment of the invention. FIG. 13A illustrates an alternative embodiment of a base ring connector 512. In an embodiment, base ring connector 512 extends longitudinally at a slant from a first base joint 1210 to a second base joint 1212. As a result, the first base joint 1210 and second base joint 1212 are offset from each other in both a longitudinal and a circumferential direction. In an alternative embodiment, base ring connector 512 may be generally straight as shown in FIG. 13A, but may extend primarily in a longitudinal direction or primarily in a circumferential direction. Thus, adjoined base joints 1210, 1212 may be offset in one or more of a longitudinal direction and a circumferential direction.

FIG. 13B illustrates an alternative embodiment of a base ring connector 512 that exhibits an accentuated curvilinear path as compared to the curvilinear path illustrated in FIG. 12B. The s-curved base ring connector 512 adjoins adjacent base joints 1210, 1212, but maintains separation between the base joints by offsetting them in a longitudinal and circumferential direction. For example, base joints 1210, 1212 may be offset such that each apex of a base joint in a first base ring 510 is approximately aligned in a circumferential direction with a base joint inner diameter 1206 of a base joint of an adjacent second base ring 510 along a longitudinal alignment axis 1300. In other words, outer curves of base joints in a first ring may mesh between outer curves of base joints in an adjacent ring. Thus, as implant 500 is crimped to an unexpanded diameter, each base joint of a first base ring 510 may fit within gaps formed between base joints of an adjacent second base ring 510, resulting in increased compaction of the base section 502 and a minimized crimped implant 500 diameter.

In an embodiment, a curvilinear path of a base ring connector 512 may be accentuated to a degree that a base joint outer diameter 1302 of a base joint 1210 in a first base ring 510 is aligned with a base joint outer diameter 1302 of a base joint 1212 in a second base ring 510 along a circumferential alignment axis 1304. Accordingly, the connected positions of adjoined base joints 1210, 1212 may be similar to an embodiment in which base ring connector 512 follows a straight circumferential path. Thus, numerous manners of interconnecting base rings 510 may be used to offset base joints and base rings in various directions.

In an embodiment, base ring connectors 512 may be staggered from ring to ring. For example, a first base ring connector 512 that interconnects a first base ring 510 with a second base ring 510 may be circumferentially offset from a second base ring connector 512 interconnecting the second base ring 510 with a third base ring 510. Accordingly, given that adjacent base joints 1100 may be circumferentially offset, base ring connectors 512 may be aligned along a helical path 1208 as shown in FIG. 12A. A helical arrangement of base ring connectors 512 may provide support along a continuous rotational load path. Circumferential offset between adjacent base rings 510 may be altered to increase or decrease the angle of helical path 1208, i.e., to make the helical path 1208 slant more circumferentially or slant more longitudinally. For example, by decreasing the circumferential offset between adjacent base rings 510 until base ring connectors 512 are aligned along a longitudinal direction, the helical path 1208 would be reduced to zero pitch and the continuous load path would be purely longitudinal. Conversely, the helical path 1208 angle may be biased toward, although it may not achieve, a purely circumferential direction by increasing the circumferential offset between adjacent base joints 1100. Thus, the continuous load path may be tuned to provide appropriate longitudinal and/or circumferential support.

In an alternative embodiment, adjacent base ring connectors 512 may be staggered from ring to ring by skipping one or more base struts 800 between interconnection points on the rings. More specifically, base ring connectors 512 from ring to ring may not interconnect at ends of a same base strut 800. Instead, there may be base struts 800 between the base ring connectors 512. In such a case, the helical path 1208 may not be continuous and may slant more in a circumferential direction. The rotational load path may thus be biased more in a circumferential direction by increasing the number of skipped base struts 800 between base joint connectors 512, e.g., from one to two or more skipped base struts 800.

In an alternative embodiment, base ring connectors 512 may be inverted relative to each other at each adjacent ring. For example, whereas a first base ring connector 512 connecting a first base ring 510 and a second base ring 510 may slant along a helical path 1208 in a clockwise direction about a longitudinal axis, a second base ring connector 512 connecting the second base ring 510 to a third base ring 510 may slant along a helical path 1208 in a counter-clockwise direction about the longitudinal axis. This inverted orientation of base ring connectors 512 from ring to ring may allow for balancing of rotational stiffness between base rings 510 to allow for implant 500 to withstand torsional loads in opposing circumferential direction.

FIG. 14A illustrates a flat pattern of a base section of a vascular implant having undulating base struts with a triple-wave design in accordance with an embodiment of the invention. In an embodiment, base struts 800 do not extend straightly between adjacent base joints 1100, but rather, extend along a curvilinear path. For example, between a first base joint 1210 and a second base joint 1212, a base strut 800 may curve through one or more waves. In an embodiment, a base strut 800 curves through three waves along an undulating path between base joints 1100. In an embodiment, an undulating path of a first base strut 800 may conform with an undulating path of an adjacent base strut 800. In combination, the conforming curvature of adjacent base struts 800 in a base ring 510 may result in a sawtooth pattern where each base joint 1100 represents a point or a trough of a tooth. In an embodiment, the teeth of base ring 510 may tend to lean in a circumferential direction, i.e., the teeth of each base ring 510 may include a rotational bias. A rotational bias as used here refers to the generally triangular tooth pattern of base ring 510 including a tooth point or apex that is biased toward longitudinal alignment with one of the base points of the tooth. In other words, when representing the tooth as a triangle, the triangle may lean to a side.

Referring to FIG. 14B, a detail view taken from Detail C of FIG. 14A, of an alternating base strut pattern of a base section of a vascular implant pattern is shown in accordance with an embodiment of the invention. In an embodiment, each base ring 510 includes an alternating strut orientation in which inverted base struts 1400 of one base ring 510 are inverted relative to base struts 800 of an adjacent base ring 510. In other words, a pattern of a base strut 800 of a first base ring 510 may be rotated 180 degrees to obtain a pattern of a corresponding inverted base strut 1400 of an adjacent second base ring 510. More specifically, first tooth 1402 element of a first base ring 510 may be rotated 180 degrees to obtain an inverted second tooth 1404 element of an adjacent second ring. Inversion of base ring 510 patterns may balance rotational bias of undulating base struts 800. For example, in an embodiment in which each adjacent base ring 510 includes a rotational bias in an opposite circumferential direction, any imbalance in base ring 510 pattern may occur during expansion. This rotational bias of teeth in a ring may be mechanically offset by reversing the bias in an adjacent ring. Thus, an alternating base ring 510 pattern may result in a more uniform expansion of base section 502 where undulating base struts 800 are utilized.

FIG. 15A illustrates a flat pattern of a base section of a vascular implant having undulating base struts with a quadruple-wave design in accordance with an embodiment of the invention. In an embodiment, each base strut 800 may curve through four waves along an undulating path between base joints 1100. Undulating paths of adjacent base struts 800 may conform with each other to allow for greater strut compaction during implant 500 crimping. In combination, the conforming curvature of adjacent base struts 800 in a base ring 510 may result in a sawtooth pattern exhibiting some degree of rotational bias. However, in an embodiment, this rotational bias may be controlled by design, e.g., a four-wave undulating base strut 800 may include less rotational bias than the three-wave undulating base strut 800 of FIG. 14A. For example, the adjacent base struts 800 may exhibit less overall curvature, resembling straight base struts 800 rather than curved base struts 800.

Referring to FIG. 15B, a detail view taken from Detail D of FIG. 15A, of a non-alternating base strut pattern of a base section of a vascular implant pattern is shown in accordance with an embodiment of the invention. In an embodiment, undulating base struts 800 do not exhibit significant rotational bias. First tooth 1402 element and second tooth 1404 element of adjacent base rings 510 may not be inverted relative to each other. That is, first tooth 1402 element and second tooth 1404 element may be similarly oriented and thus each base ring 510 may include similar patterns.

FIG. 16A illustrates a flat pattern of a zig-zag aneurysm pattern of a vascular implant in an expanded state in accordance with an embodiment of the invention. In an embodiment, aneurysm section holders 514 support a plurality of aneurysm arcs 518 that form aneurysm pattern 516. Aneurysm pattern 516 may be referred to as a zig-zag pattern in an embodiment in which aneurysm arcs 518 extend in a primarily circumferential direction with little or no curvature as the arc traverses between aneurysm section holders 514. A zig-zag pattern may be more accommodative to expansion of aneurysm pattern 516 toward a longitudinal cylindrical segment contour, since the arcs will approximate a portion of a cylinder and provide uniform scaffolding across an aneurysm gate.

In an embodiment, aneurysm pattern 516 may include end arcs 1600 connected to aneurysm section holders 514 near a distal or proximal location. Furthermore, a plurality of medial arcs 1602 may be connected with aneurysm section holder 514 along an inner surface of the arcuate path the holders traverse. In an embodiment, each aneurysm arc 518 extends radially in a substantially circumferential direction between aneurysm section holders 514, forming an arc in contrast to the rings formed by base rings 510. That is, aneurysm arcs 518 forming aneurysm pattern 516 may be circumferentially discontinuous as compared to the circumferentially continuous structure of base rings 510. In an expanded state, aneurysm arcs 518 may therefore be considered to extend along a substantially non-circumferential path. In alternative embodiments, aneurysm arcs 518 may extend in a slanted and or primarily longitudinal direction across aneurysm pattern 516. For example, aneurysm arcs may extend in a generally helical direction from a first aneurysm section holder 514 to a second aneurysm section holder 514. Furthermore, in alternative embodiments, aneurysm arcs 518 may be connected with only one aneurysm section holder 514. For example, aneurysm arcs 518 may be arranged in a cross-hatch pattern across aneurysm pattern 516 in which aneurysm arcs originating at one aneurysm section holder 514 extend and meet with aneurysm arcs originating from another aneurysm section holder 514. Thus, each aneurysm arcs may only be connected with aneurysm section holders 514 at one end. Therefore, the pattern shown in FIG. 16A is illustrative and not limiting.

End arcs 1600 and medial arcs 1602 may include numerous aneurysm struts 1604 and aneurysm joints 1606 with mechanical similarities to base struts 800 and base joints 1100 of base section 502. However, the design of aneurysm struts 1604 of end arc 1600 may also differ substantially from aneurysm strut 1604 of medial arc 1602. For example, in an embodiment, end arc 1600 may include aneurysm struts 1604 with lengths roughly twice that of aneurysm struts 1604 in medial arc 1602. However, aneurysm struts 1604 of end arc 1600 and aneurysm struts 1604 of medial arc 1602 may be more closely matched in length. For example, in an embodiment, medial struts of medial arcs 1602 may be equal, slightly longer, or even shorter than struts of end arcs 1600. For example, aneurysm struts 1604 of end arcs 1600 may include a length of about 0.05-inch while aneurysm struts 1604 of medial arcs 1602 may be about 0.03-inch long. In another embodiment, aneurysm struts 1604 of end arcs may be about 0.06-inch in length while aneurysm struts 1604 of medial arcs 1602 may be about 0.07-inch long. Similarly, the number of aneurysm joints 1606 in end arc 1600 may differ from the number of aneurysm joints 1606 in medial arc 1602. For example, in an embodiment, end arcs 1600 include four aneurysm joints 1606 along a side and one or more medial arcs 1602 include eight aneurysm joints 1606 along a side. However, this number may vary and in an embodiment, end arcs 1600 may include three aneurysm joints 1606 along a side and one or more medial arcs 1602 may include six aneurysm joints along a side. Similarly, aneurysm strut 1604 and aneurysm joint 1606 designs may vary between individual arc sections, such as amongst medial arcs 1602. In an embodiment, a centermost medial arc 1602 may include more aneurysm joints 1606 than a medial arc 1602 distal or proximal thereof, given that the centermost medial arc 1602 traverses a greater distance between the apices of aneurysm section holders 514. Alternatively, aneurysm strut 1604 lengths or angles between aneurysm struts 1604 of a medial arc 1602 may be varied to permit the same number of aneurysm joints 1606 to be used for all medial arcs 1602 even though the medial arcs 1602 extend over different distances between aneurysm section holders 514. It will be appreciated that the quantities of pattern features provided above are examples, and may be varied within the scope of the invention. For example, in an embodiment, there may be one a single medial arc and/or only a single end arc. Furthermore, aneurysm pattern 516 may include only a single type of aneurysm arc 518, and may include one or more of such arcs.

In an embodiment, aneurysm section 504 may have a scaffolding coverage that is either higher, or similar to, scaffolding coverage of base section 502. In addition, the ratio of scaffolding coverage may vary within aneurysm pattern 516, e.g., medial arc area of aneurysm section 504 may have greater scaffolding coverage than that of end arc area of aneurysm section 504. For example, more medial arcs 1602 or a denser arc pattern may be used in a medial arc region of aneurysm section 504 than in an end arc region of aneurysm section 504. In an embodiment, an increase in pattern density of aneurysm section 504 may be facilitated by including medial arc 1602 with strut width dimensions that are less than width dimensions of other struts, e.g., in base section 502. Narrower struts may permit more struts to be included in a region area, and thus facilitate higher strut pattern density. In an embodiment, medial arcs 1602 and/or end arcs 1600 in aneurysm section 504 include aneurysm strut widths in a range of about 0.0010-inch to 0.0015-inch. More particularly, aneurysm struts 1604 may have a width of about 0.0012-inch.

In an embodiment, aneurysm arcs 518 are interconnected by aneurysm pattern connectors 520. Aneurysm pattern connectors 520 may vary depending upon the aneurysm arcs 518 that are being coupled. For example, aneurysm pattern connectors 520 that interconnect end arcs 1600 with medial arcs 1602 may include several undulations, e.g., an s-curved path. The undulations may provide a highly flexible structure to accommodate, for example, the bulging of a medial arc 1602 into an aneurysm sac while an adjoined end arc 1600 remains pressed against an aneurysm gate. Alternatively, aneurysm pattern connectors 520 may include fewer or no undulations. Here, undulations may refer to any curve, zag, or other deviation from a straight configuration. For example, aneurysm pattern connectors 520 interconnecting adjacent medial arcs 1602 may include a single u-shaped path. Such aneurysm pattern connectors 520 may provide for some degree of longitudinal movement between arcs while maintaining an even contour across the surface of the medial arcs 1602. Any combination or alteration of aneurysm pattern connectors 520 may be made to accommodate different arc designs, and the examples provided are illustrative and not limiting. In still other embodiments, aneurysm pattern connectors 520 are optional. That is, in an embodiment, aneurysm arcs 518 may not be connected by connectors, but may instead by physically separated to expand independently.

FIG. 16B illustrates a flat pattern of a zig-zag aneurysm pattern of a vascular implant in an unexpanded state in accordance with an embodiment of the invention. In an embodiment, a flattened representation of aneurysm pattern 516 transitions from having a generally circular profile in an expanded state to having a generally elliptical profile in an unexpanded state. Furthermore, the distance between aneurysm section holders 514 in a circumferential direction may be significantly shortened by, e.g., about four times. As an example, a circumferential distance across a medial arc 1602 may measure about 5.5 mm in an expanded state and only about 1.4 mm in an unexpanded state. To accommodate this change in profile shape and dimension, aneurysm joints 1606 articulate to allow aneurysm struts 1604 to stack closely together. In addition, aneurysm pattern connectors 520 may align with gaps between aneurysm joints 1606 of adjacent aneurysm arcs 518 to facilitate the close stacking of aneurysm struts 1604. For example, there may be a pattern gap 1608 of at least about 0.001-inch, e.g., about 0.005-inch, between adjacent aneurysm arcs to receive aneurysm pattern connectors 520 in the unexpanded state. Accordingly, a tightly packed aneurysm pattern 516 may be reached, which when wrapped about a longitudinal axis of implant 500 forms a substantially cylindrical contour. More particularly, one or more aneurysm arcs 518 may extend along a substantially circumferential path in the unexpanded state.

FIG. 17A illustrates a flat pattern of a parallelogram aneurysm pattern 516 of a vascular implant 500 in an expanded state in accordance with an embodiment of the invention. Aneurysm pattern 516 may be referred to as a parallelogram pattern in an embodiment in which aneurysm arcs 518 extend along an arcuate path 1700 between aneurysm section holders 514. A parallelogram pattern may be more accommodative to expansion of aneurysm pattern 516 toward a bulbous contour that protrudes into an aneurysm sac, since the arcuate arcs will approximate circumferential lines of a sphere, e.g., latitudinal lines of a sphere. Depending upon the degree of targeted protrusion, the radius of arcuate path 1700 may be adjusted to allow for the medial arcs 1602 to evenly spread around the bulbous contour, thus creating uniform scaffolding within the aneurysm sac.

In an embodiment, parallelogram aneurysm pattern 516 includes an even number of medial arcs 1602. An even number of medial arcs 1602 may allow for equal numbers of medial arcs 1602 to be located proximal and distal to a geometric plane passing through the apices of aneurysm section holders 514. For example, parallelogram aneurysm pattern 516 may include two medial arcs 1602 proximal to the apex and two medial arcs 1602 distal to the apex. Aneurysm pattern connectors 520 may interconnect adjacent medial arcs 1602 at aneurysm joints 1606. In an embodiment, aneurysm pattern connectors 520 constrain expansion of adjacent medial arcs 1602 relative to each other such that there will not be abrupt expansion of one arc and under expansion of an adjacent arc. In other words, aneurysm pattern connectors 520 contribute to a smoother surface of an expanded bulbous contour of aneurysm pattern 516. Aneurysm pattern connectors 520 may be sized and configured according to any of the connector embodiments described above, including undulating, s-shaped, u-shaped, z-shaped, slanted, straight, etc. It will be appreciated that, as described above, aneurysm pattern connectors 520 are optional, and parallelogram aneurysm pattern 516 may include arcs that are unconnected and expand independently. Thus, various embodiments of aneurysm patterns may include medial arcs unconnected with each other but connected with end arcs, medial arcs connected with each other but not connected with end arcs, end arcs unconnected with each other but connected with medial arcs, end arcs connected with each other but not connected with medial arcs, and any other combination of arc connections across aneurysm pattern 516.

Optionally, aneurysm pattern 516 may include end arcs 1600. End arcs 1600 may expand toward an aneurysm gate as medial arcs 1602 protrude into an aneurysm sac. However, in at least one embodiment, end arcs 1600 may not be interconnected with medial arcs 1602 by aneurysm pattern connectors 520. Thus, in an embodiment, end arcs 1600 and medial arcs 1602 may expand independently from each other.

FIG. 17B illustrates a flat pattern of a parallelogram aneurysm pattern of a vascular implant in an unexpanded state in accordance with an embodiment of the invention. As discussed with respect to FIGS. 16A-16B, aneurysm pattern 516 may transition between a generally circular profile in an expanded state to a generally elliptical profile in an unexpanded state. Thus, in the unexpanded state, aneurysm pattern 516 may be wrapped about a longitudinal axis of implant 500 to assume a substantially cylindrical contour. Furthermore, end arcs 1600 and medial arcs 1602 may be tightly packed between aneurysm section holders 514 in the unexpanded state. Close stacking of aneurysm struts 1604 may be facilitated by providing gaps between adjacent rings within which aneurysm pattern connectors 520 may fit. Furthermore, in a parallelogram aneurysm pattern, medial arcs 1602 may stack along arcuate path 1700 in the unexpanded state. As described above, aneurysm pattern 516 may expand in a circumferential direction less than a corresponding contour of base section 502, and thus, may exhibit a denser scaffold area after expansion.

As mentioned above, aneurysm pattern 516 may have numerous designs within the scope of the invention. More particularly, the patterns described above with respect to zig-zag and parallelogram aneurysm patterns are illustrative and not limiting. In addition to the embodiments described above, FIGS. 22A-22G below provide several additional embodiments of implant 500 patterns with various aneurysm patterns 516.

FIG. 18A illustrates a flat pattern of a transition ring of a vascular implant pattern in accordance with an embodiment of the invention. In an embodiment, base section 502 includes a transition ring 1800 between aneurysm section 504 and an adjacent base ring 510. Transition ring 1800 may be considered to be a base ring 510, although it may include a pattern and manner of interconnection that is different from other base rings 510 in the base subsection within which it is located. For example, whereas an adjoining base ring 510 may include six base joints 1100 on a side, transition ring 1800 may include seven base joints 1100 on a side. A transition ring 1800 with seven base joints 1100 on a side of transition ring 1800 may allow for one unattached base joint 1100 to be positioned between each base joint 1100 that attaches to an aneurysm section holder 514 or aneurysm connector 522. For example, in a case where implant 500 includes a total of four aneurysm section holders 514 and aneurysm connectors 522, and base section 502 to which the holder and connector ends attach includes a total of seven base joints 1100, holders and connector ends may be attached to every other base joint 1100 such that a single unattached base joint 1100 is provided between each holder and connector and therefore the aneurysm connectors 522 and aneurysm section holders 514 are uniformly distributed around the circumference of aneurysm section 504. Thus, transition ring 1800 may be altered relative to other base rings 510 to allow for even distribution of aneurysm section holders 514 and aneurysm connectors 522 around implant 500 circumference. Additionally, an increased number of base joints 1100 in transition ring 1800 may allow for transition ring 1800 base joints 1100 to expand to a degree that supports aneurysm section holders 514 while allowing aneurysm pattern 516 to reach an expanded diameter that covers an entire aneurysm gate, e.g., a diameter of about 5.5 mm to 10 mm. This expansion diameter is provided as an example since aneurysm pattern 516 may be varied within the scope of this invention to accommodate larger or smaller aneurysm gate diameters. For example, in some embodiments, aneurysm pattern 516 may expand to cover an aneurysm gate diameter of more than 10 mm.

In an embodiment, transition ring 1800 may be adjoined with base ring 510 by one or more transition ring connectors 1802. For example, transition ring connector 1802 may extend from a base joint 1100 of base ring 510 toward a base joint 1100 of transition ring 1800. As with other base ring connectors 512, transition ring connector 1802 may extend in a generally helical direction to offset adjacent base joints 1100 in a longitudinal and/or circumferential direction. In an embodiment, a second transition ring connector 1804 may extend from base ring 510 in an opposite direction from transition ring connector 1802. For example, transition ring connector 1802 may extend helically in a clockwise direction relative to a longitudinal axis while second transition ring connector 1804 may extend helically in a counter-clockwise direction relative to a longitudinal axis. Opposing directions of extension of transition ring connector 1802 and second transition ring connector 1804 may promote structural balance and support torsional loads in opposing directions.

In an embodiment, transition ring 1800 may be connected directly to aneurysm section holders 514. For example, aneurysm section holders 514 may extend directly in a longitudinal direction from a base joint 1100 or a base strut 800 of transition ring 1800. In an alternative embodiment, aneurysm section holders 514 may extend first in a circumferential direction or slanted direction from transition ring 1800 prior to extending in a longitudinal direction. Furthermore, in an alternative embodiment, connectors such as transition ring connectors 1802 may be used to adjoin transition ring 1800 to aneurysm section holders 514. Thus, for example, an s-shaped connector may adjoin a base joint 1100 of transition ring 1800 to an end of an aneurysm section holder 514 in order to provide greater flexibility between transition ring 1800 and aneurysm section holder 514. In other words, features may be introduced to enhance flexibility in the implant structure in the vicinity where aneurysm section holders 514 meet base section 502.

Aneurysm section holders 514 and aneurysm connectors 522 may include other features to further increase flexibility in bending near an interconnection with transition ring 1800. For example, a recess or notch feature may be used to increase flexibility. More specifically, in an embodiment, aneurysm section holder 514 may be notched where it meets a base joint 1100 to reduce cross-sectional area at the location and thereby lower structural stiffness and increase flexibility. In other words, the local stiffness of aneurysm section holders 514 may be varied to enhance flexibility in the implant structure in the vicinity where aneurysm section holders 514 meet base section 502.

Referring to FIG. 18B, a detail view taken from Detail E of FIG. 18A, of a transition ring connector of a vascular implant pattern is shown in accordance with an embodiment of the invention. In an embodiment, transition ring connector 1802 extends straightly in a helical direction between base joint 1100 of base ring 510 and base joint 1100 of transition ring 1800. However, transition ring connector 1802 may be formed in any of the manners described above, including with an undulating, s-shaped, u-shaped, z-shaped, or slanted shape. Furthermore, whereas transition ring connector 1802 may adjoin with base joints 1100 near base joint 1100 apices, in another embodiment, transition ring connectors 1802 adjoin base joints 1100 and/or base struts 800 at a location lateral, i.e., offset from, the apices.

Referring again to FIGS. 12A, 14A, and 15A, implant 500 may include numerous markers that facilitate visualization and placement of the implant 500 during and after delivery into a patient. For example, markers may be provided to indicate the ends of the implant 500. Alternatively, markers may be provided to indicate the location of a particular feature of the implant 500, such as the aneurysm pattern 516. Markers may be fabricated for detection under a particular imaging modality. For example, in an embodiment, markers are formed from a radiopaque material such as a noble metal, e.g., platinum, gold, silver, and palladium to facilitate visualization under fluoroscopy. When selecting a material, it may be important to consider the implant material. For example, noble metals may be suitable choices for an implant formed from a balloon-expandable material such as stainless steel, or cobalt chrome alloys. However, if implant is formed from a self-expandable material such as superelastic nickel titanium, tantalum may be a more suitable marker material due to the similarity of the metals, which may enhance corrosion resistance.

Referring to FIG. 19, a side view of an end marker of a vascular implant is shown in accordance with an embodiment of the invention. End markers 1204 may be located near implant 500 ends within an end marker holder 1900. End marker holder 1900 may be integrally formed with a base ring such as end ring 1202. For example, end marker holder 1900 may be laser cut along with base ring 510 and extend away from base joint 1100 with a profile that encloses a marker area. Thus, in an embodiment in which each base joint of end ring 1202 adjoins with an end marker holder 1900, implant 500 may include six end markers 1204 at each end, for a total of twelve end markers 1204. End markers 1204 provide an indication of the ends of implant 500 to facilitate accurate delivery and deployment of implant 500 as described below. In an embodiment, end markers 1204 are triangularly shaped, however any marker shape may be used with sufficient marker material to provide visibility under a chosen imaging modality.

Referring to FIG. 20A, a side view of an aneurysm marker near a medial location of an aneurysm section holder of a vascular implant is shown in accordance with an embodiment of the invention. Aneurysm markers 2002 may be provided at one or more location along aneurysm section holders 514 to demarcate a perimeter of aneurysm pattern 516. For example, an aneurysm marker holder 2000 may be located at lateral apex 1102 of aneurysm section holder 514. In an embodiment, aneurysm marker holder 2000 biases toward the aneurysm pattern-side of aneurysm section holder 514. In other embodiments, aneurysm marker 2002 may instead be biased toward the aneurysm connector-side of aneurysm section holder 514, or it may be located in the middle of aneurysm section holder 514. Aneurysm markers 2002 near lateral apex 1102 of aneurysm section holders 514 provide an indication of the middle of aneurysm pattern 516 to facilitate accurate delivery and deployment of implant 500 as described below. In an embodiment, aneurysm markers 2002 are elliptically shaped, however any marker shape may be used with sufficient marker material to provide visibility under a chosen imaging modality.

Referring to FIG. 20B, a side view of an aneurysm marker near a base location of an aneurysm section holder of a vascular implant is shown in accordance with an embodiment of the invention. In addition to being placed near lateral apex 1102 of aneurysm section holder 514, one or more aneurysm markers 2002 may be longitudinally spaced along aneurysm section holder 514. For example, each aneurysm section holder 514 may include an aneurysm marker 2002 located near either end and near lateral apex 1102. Thus, in an embodiment, each aneurysm section holder 514 includes three aneurysm marker 2002 evenly spaced along aneurysm pattern length, for a total of six aneurysm markers 2002. Aneurysm markers 2002 along the length of aneurysm section holders 514 indicate a perimeter of aneurysm pattern 516 to facilitate accurate delivery and deployment of implant 500 as described below. The quantity of aneurysm markers 2002 may be varied accordingly, and thus, the quantity of six aneurysm markers 2002 is provided above as an example only.

Visibility of implant markers such as end markers 1204 and aneurysm markers 2002 may be directly correlated with the size of the markers. More specifically, the greater the volume and/or thickness of the markers, the more visible the markers may be under fluoroscopy. In an embodiment, markers may be at least as thick as marker holders. For example, in the case of end marker holder 1900 having a thickness approximately equal to that of an adjacent base ring 510, an end marker 1204 may have a thickness of about 0.0024-inch. Moreover, end marker holder 1900 area may be in a range of about 0.00004 to 0.00005 square inch. Thus, a marker volume may be about 10×10⁻⁸ to 12×10⁻⁸ cubic inches. Aneurysm markers 2002 may be sized similar to end markers 1204 to ensure consistent visualization of all markers under similar imaging parameters.

End markers 1204 and aneurysm markers 2002 may be positioned and fixed within marker holders using various manufacturing process, such as stamping, press fitting, adhesive or thermal welding. Alternatively or in combination with bonding processes, markers may be press fit within marker holders. For example, a slug of radiopaque material may be loaded into a marker holder and then stamped until it deforms into apposition with the marker holder. In an alternative embodiment, other processes may be used to load implant 500 with a radiopaque material, such as coating, sputtering, or other known surface treatment processes.

It will be appreciated that the module design of implant 500 allows for the above described structural features to be combined in numerous manners without departing from the scope of the invention. For example, in numerous alternative embodiments, structural features such as transition ring 1800 may be included or omitted from the implant pattern. Similarly, the number of base joints 1100 between aneurysm section holders 514 attachment to base section 502 may be altered in various embodiments. Further still, the length of aneurysm pattern 516 may be varied, for example but not by limitation, between about 5 to 10 mm. FIGS. 21A-21J illustrate flat patterns of numerous implant 500 configurations that combine structural features in various manners in accordance with an embodiment of the invention.

FIG. 21A illustrates implant 500 having aneurysm pattern 516 with a total of six aneurysm arcs 518 interconnected by one or more aneurysm pattern connectors 520. In an embodiment, aneurysm pattern length 2102 measures about 10 mm. However, in other embodiments, this length may vary based on the number of aneurysm arcs 518 in aneurysm pattern 516. Additionally, transition ring 1800 may include seven base joints 1100 along either side and, for example, one base joint 1100 may be between aneurysm section holders 514 where aneurysm section holders 514 join transition ring 1800.

FIG. 21B illustrates implant 500 having aneurysm pattern 516 with a total of three aneurysm arcs 518 interconnected by one or more aneurysm pattern connectors 520. In an embodiment, aneurysm pattern length 2102 measures about 5.5 mm. Additionally, transition ring 1800 may include seven base joints 1100 along either side and, for example, three base joints 1100 may be between aneurysm section holders 514 where aneurysm section holders 514 join transition ring 1800. Accordingly, there may be no base joints 1100 between aneurysm section holders 514 and aneurysm connectors 522. Thus, aneurysm section holders 514 and aneurysm connectors 522 may be unevenly distributed around a circumference of transition ring 1800.

FIG. 21C illustrates implant 500 having aneurysm pattern 516 with a total of six aneurysm arcs 518 interconnected by one or more aneurysm pattern connectors 520. In an embodiment, aneurysm pattern length 2102 measures about 10 mm. Additionally, transition ring 1800 may include seven base joints 1100 along either side and, for example, three base joints 1100 may be between aneurysm section holders 514 where aneurysm section holders 514 join transition ring 1800. In an embodiment, whereas aneurysm section holders 514 may be separated from each other circumferentially by three base joints 1100, there may be no base joints separating aneurysm section holders 514 from adjacent aneurysm connectors 522. More particularly, aneurysm connectors 522 may connect with a base joint 1100 of transition ring 1800 that is immediately adjacent to another base joint 1100 connected with aneurysm section holder 514.

FIG. 21D illustrates implant 500 having aneurysm pattern 516 with a total of three aneurysm arcs 518 interconnected by one or more aneurysm pattern connectors 520. In an embodiment, aneurysm pattern length 2102 measures about 5.5 mm. Additionally, implant 500 may not include transition ring 1800. More particularly, base ring 510 may interconnect an adjacent base ring 510 with aneurysm section holders 514. Furthermore, base ring 510 connected with aneurysm section holders 514 may include six base joints 1100 along either side and, for example, two base joints 1100 may be between aneurysm section holders 514 where aneurysm section holders 514 join base ring 510. In an embodiment, whereas aneurysm section holders 514 may be separated from each other circumferentially by two base joints, there may be no base joints separating aneurysm section holders 514 from adjacent aneurysm connectors 522. More particularly, aneurysm connectors 522 may connect with a base joint 1100 of base ring 510 that is immediately adjacent to another base joint 1100 connected with aneurysm section holder 514.

FIG. 21E illustrates implant 500 having aneurysm pattern 516 with a total of six aneurysm arcs 518 interconnected by one or more aneurysm pattern connectors 520. In an embodiment, aneurysm pattern length 2102 measures about 10 mm. Additionally, implant 500 may not include transition ring 1800. More particularly, base ring 510 may interconnect an adjacent base ring 510 with aneurysm section holders 514. Furthermore, base ring 510 connected with aneurysm section holders 514 may include six base joints 1100 along either side and, for example, two base joints 1100 may be between aneurysm section holders 514 where aneurysm section holders 514 join base ring 510. In an embodiment, whereas aneurysm section holders 514 may be separated from each other circumferentially by two base joints, there may be no base joints separating aneurysm section holders 514 from adjacent aneurysm connectors 522. More particularly, aneurysm connectors 522 may connect with a base joint 1100 of base ring 510 that is immediately adjacent to another base joint 1100 connected with aneurysm section holder 514.

FIG. 21F illustrates implant 500 having aneurysm pattern 516 with a total of six aneurysm arcs 518. In an embodiment, aneurysm arcs 518 are not interconnected by one or more aneurysm pattern connectors 520. That is, aneurysm arcs 518 may only be connected with aneurysm section holders 514, permitting aneurysm arcs 518 to expand independently from one another. In an embodiment, aneurysm pattern length 2102 measures about 10 mm. Additionally, transition ring 1800 may include seven base joints 1100 along either side and, for example, one base joint 1100 may be between aneurysm section holders 514 where aneurysm section holders 514 join transition ring 1800.

FIG. 21G illustrates implant 500 having aneurysm pattern 516 with a total of three aneurysm arcs 518, which are not interconnected by one or more aneurysm pattern connectors 520. In an embodiment, aneurysm pattern length 2102 measures about 5.5 mm. Additionally, transition ring 1800 may include seven base joints 1100 along either side and, for example, three base joints 1100 may be between aneurysm section holders 514 where aneurysm section holders 514 join transition ring 1800. In an embodiment, whereas aneurysm section holders 514 may be separated from each other circumferentially by three base joints 1100, there may be no base joints separating aneurysm section holders 514 from adjacent aneurysm connectors 522. More particularly, aneurysm connectors 522 may connect with a base joint 1100 of transition ring 1800 that is immediately adjacent to another base joint 1100 connected with aneurysm section holder 514.

FIG. 21H illustrates implant 500 having aneurysm pattern 516 with a total of six aneurysm arcs 518, which are not interconnected by one or more aneurysm pattern connectors 520. In an embodiment, aneurysm pattern length 2102 measures about 10 mm. Additionally, transition ring 1800 may include seven base joints 1100 along either side and, for example, three base joints 1100 may be between aneurysm section holders 514 where aneurysm section holders 514 join transition ring 1800. In an embodiment, whereas aneurysm section holders 514 may be separated from each other circumferentially by three base joints 1100, there may be no base joints separating aneurysm section holders 514 from adjacent aneurysm connectors 522. More particularly, aneurysm connectors 522 may connect with a base joint 1100 of transition ring 1800 that is immediately adjacent to another base joint 1100 connected with aneurysm section holder 514.

FIG. 21I illustrates implant 500 having aneurysm pattern 516 with a total of three aneurysm arcs 518, which are not interconnected by one or more aneurysm pattern connectors 520. In an embodiment, aneurysm pattern length 2102 measures about 5.5 mm. Additionally, implant 500 may not include transition ring 1800. More particularly, base ring 510 may interconnect an adjacent base ring 510 with aneurysm section holders 514. Furthermore, base ring 510 connected with aneurysm section holders 514 may include six base joints 1100 along either side and, for example, two base joints 1100 may be between aneurysm section holders 514 where aneurysm section holders 514 join base ring 510. In an embodiment, whereas aneurysm section holders 514 may be separated from each other circumferentially by two base joints, there may be no base joints separating aneurysm section holders 514 from adjacent aneurysm connectors 522. More particularly, aneurysm connectors 522 may connect with a base joint 1100 of base ring 510 that is immediately adjacent to another base joint 1100 connected with aneurysm section holder 514.

FIG. 21J illustrates implant 500 having aneurysm pattern 516 with a total of six aneurysm arcs 518, which are not interconnected by one or more aneurysm pattern connectors 520. In an embodiment, aneurysm pattern length 2102 measures about 10 mm. Additionally, implant 500 may not include transition ring 1800. More particularly, base ring 510 may interconnect an adjacent base ring 510 with aneurysm section holders 514. Furthermore, base ring 510 connected with aneurysm section holders 514 may include six base joints 1100 along either side and, for example, two base joints 1100 may be between aneurysm section holders 514 where aneurysm section holders 514 join base ring 510. In an embodiment, whereas aneurysm section holders 514 may be separated from each other circumferentially by two base joints, there may be no base joints separating aneurysm section holders 514 from adjacent aneurysm connectors 522. More particularly, aneurysm connectors 522 may connect with a base joint 1100 of base ring 510 that is immediately adjacent to another base joint 1100 connected with aneurysm section holder 514.

As mentioned above, it will be appreciated that the modular design of implant 500 allows for aneurysm pattern 516 to be altered in numerous manners without departing from the scope of this invention. For example, rather than expanding from an elliptical profile toward a circular profile, aneurysm pattern 516 may be rectangular, triangular, etc. FIGS. 22A-22G illustrate flat patterns of numerous aneurysm patterns 516 of a vascular implant 500 in an unexpanded state in accordance with an embodiment of the invention.

FIG. 22A illustrates a flat pattern illustration of an alternative embodiment of a vascular implant 500 having a set of circular concentric struts 2202 forming an aneurysm pattern 516 in accordance with an embodiment of the invention. Implant 500 includes similar modular components to those described above, e.g., aneurysm section 504 and base section 502. Additionally, aneurysm section 504 includes an aneurysm scaffold support 2200 opposite of aneurysm section holders 514 from aneurysm pattern 516. In an embodiment, aneurysm scaffold support 2200 includes one or more radial stent arcs that may be expanded opposite from aneurysm pattern 516 to scaffold a parent vessel and provide radial support to aneurysm pattern 516 placed against an aneurysm gate. The stent arc design may include any of the features described above with respect to base rings 510 and/or aneurysm arcs 518. Additionally, aneurysm scaffold support 2200 may be configured to expand circumferentially more than aneurysm pattern 516. Thus, after expansion of implant 500 at an aneurysm site, scaffolding of parent vessel by aneurysm scaffold support 2200 may be less dense than scaffolding of aneurysm gate by aneurysm pattern 516.

Aneurysm pattern 516 may include a set of circular concentric struts 2202 originating at a hub 2204 and oscillating in a switchback fashion toward a perimeter of aneurysm pattern 516. For example, in an embodiment, four circular concentric struts 2202 extend radially from hub 2204 and switchback within four separate quadrants of aneurysm pattern 516. The circular concentric struts 2202 may not be constrained to a particular quadrant, but may cross over into adjacent quadrants. Furthermore, the struts may mesh within the various quadrants to form a more dense scaffold within aneurysm pattern 516 than within other portions of implant 500.

FIG. 22B illustrates a flat pattern illustration of an alternative embodiment of a vascular implant 500 having a screened aneurysm pattern 516 in accordance with an embodiment of the invention. In an embodiment, aneurysm pattern 516 includes a single screen strut 2206 extending radially from aneurysm section holders 514. Screen strut 2206 oscillates in a longitudinal direction radially between aneurysm section holders 514 to form aneurysm pattern 516. In an embodiment, connectors are used to interconnect screen strut 2206 of aneurysm pattern 516 with transition rings 1800. Thus, as aneurysm pattern 516 expands toward aneurysm gate, screen strut 2206 may expand toward a zig-zag pattern that scaffolds aneurysm gate. As with other embodiments, aneurysm pattern 516 includes a more densely packed strut pattern than other portions of implant 500.

FIG. 22C illustrates a flat pattern illustration of an alternative embodiment of a vascular implant 500 having a set of strut blocks 2208 forming an aneurysm pattern 516 in accordance with an embodiment of the invention. Aneurysm pattern 516 may include a plurality of strut blocks 2208 arranged together in a generally circular pattern. Each strut block 2208 may include one or more struts that originate either at a hub 2204 of aneurysm pattern 516 or along perimeter of aneurysm pattern 516. In an embodiment, six strut blocks 2208 are arranged about hub 2204. Two strut blocks 2208 may include a single strut that passes through strut block 2208 from hub 2204 to a perimeter of aneurysm pattern 516. Four of the six strut blocks 2208 may include a first strut 2210 that originates at hub 2204 and passes through strut block 2208 without joining the perimeter at an outer edge of the strut block 2208. Those four strut blocks 2208 may include a second strut 2212 that originates at the perimeter of aneurysm pattern 516 and joins with first strut 2210 near a middle of strut block 2208. As with other embodiments, aneurysm pattern 516 includes a more densely packed strut pattern than other portions of implant 500.

FIG. 22D illustrates a flat pattern illustration of an alternative embodiment of a vascular implant 500 having a rectangular aneurysm pattern 516 in accordance with an embodiment of the invention. In an embodiment, aneurysm pattern 516 includes a rectangular pattern including a plurality of individual strut cones 2214. Each strut cone 2214 may be joined to one or more adjacent strut cones 2214 at a plurality of junctions 2216. Accordingly, in an embodiment, aneurysm pattern 516 expands toward a rectangular profile. Nonetheless, in an embodiment, the contour of aneurysm pattern 516 with a rectangular profile may be either cylindrically segmental or bulbous, as described above. As with other embodiments, aneurysm pattern 516 includes a more densely packed strut pattern than other portions of implant 500.

FIG. 22E illustrates a flat pattern illustration of an alternative embodiment of a vascular implant 500 having nodules 2218 incorporated into aneurysm pattern 516 in accordance with an embodiment of the invention. In an embodiment, aneurysm pattern 516 includes a single screen strut 2206 extending radially from aneurysm section holders 514. Screen strut 2206 oscillates in a longitudinal direction radially between aneurysm section holders 514 to form aneurysm pattern 516. In an embodiment, connectors are used to interconnect screen strut 2206 of aneurysm pattern 516 with transition rings 1800. Thus, as aneurysm pattern 516 expands toward aneurysm gate, screen strut 2206 may expand toward a zig-zag pattern that scaffolds aneurysm gate. In addition, various nodules 2218 may be located along screen strut 2206. Nodules 2218 may be shaped to mesh with each other, thereby creating a substantially solid scaffold pattern in an unexpanded state. Furthermore, as aneurysm pattern 516 expands, nodules 2218 cover more surface area than a thinner strut of uniform width. Accordingly, as with other embodiments, aneurysm pattern 516 includes a more densely packed strut pattern than other portions of implant 500.

FIG. 22F illustrates a flat pattern illustration of an alternative embodiment of a vascular implant 500 having a curved continuous strut aneurysm pattern 516 in accordance with an embodiment of the invention. Aneurysm pattern 516 may include one or more curved continuous struts radiating from hub 2204 toward an outer perimeter. For example, in an embodiment, four spiral struts 2220 originate at hub 2204 and radiate continuously in a spiral fashion toward an outer perimeter at either aneurysm section holders 514 or transition rings 1800. As with other embodiments, aneurysm pattern 516 includes a more densely packed strut pattern than other portions of implant 500.

FIG. 22G illustrates a flat pattern illustration of an alternative embodiment of a vascular implant 500 having a set of semicircle blocks 2222 forming an aneurysm pattern 516 in accordance with an embodiment of the invention. Aneurysm pattern 516 may include a plurality of semicircle blocks 2222 arranged together in a generally circular pattern. Each semicircle block 2222 may include one or more struts that originate either at hub 2204 of aneurysm pattern 516 or along perimeter of aneurysm pattern 516. In an embodiment, two semicircle blocks 2222 are arranged about hub 2204. Each semicircle block 2222 may include a first strut 2210 that originates at hub 2204 and oscillates toward aneurysm section holder 514 lateral to hub 2204. Each semicircle block 2222 may also include a second strut that originates at the perimeter of aneurysm pattern 516 at a transition ring 1800 and oscillates inward to connect with the first strut 2210 near a middle of semicircle block 2222. As with other embodiments, aneurysm pattern 516 includes a more densely packed strut pattern than other portions of implant 500.

Each of the embodiments illustrated in FIGS. 22A-22G show aneurysm pattern 516 as occupying only about one-half to one-third of the entire circumference of implant 500. Accordingly, in an embodiment, aneurysm pattern 516 may not extend substantially around a circumference of an unexpanded implant 500. Nonetheless, in each of the embodiments described above, aneurysm pattern 516 includes a scaffolding area that is greater in an expanded state than a corresponding scaffolding area of base section 502. More particularly, in an embodiment, aneurysm pattern 516 and base section 502 may include a scaffolding coverage to surface area ratio that varies between an unexpanded state and in an expanded state. More particularly, as aneurysm pattern 516 and base section 502 expand, e.g., during implant 500 deployment, the ratio reduces since the patterns making up those sections will cover more area, thus increasing the denominator of the ratio. However, in an embodiment, the expanded surface area coverage of aneurysm pattern 516 is less than the expanded surface area coverage of a corresponding area of base section 502. Thus, aneurysm pattern 516 expands less than base section 502, and accordingly, an expanded scaffold coverage to surface area ratio of aneurysm pattern 516 is higher in aneurysm pattern 516 than in a corresponding area of base section 502.

The description above relates primarily to various embodiments of structural features of an implant 500 for treating an aneurysm. These structural features are not necessarily specific to a particular implant material. Thus, implant 500 may be formed using a variety of materials. In an embodiment, implant 500 may be formed from materials that are suited to expansion using a balloon-expandable delivery system. For example, implant 500 may be formed from stainless steel alloys, e.g., series 316L stainless steel, cobalt chrome alloys, e.g., L605 cobalt chrome or Elgiloy, MP35N, or platinum chrome, to name a few. Alternatively, implant 500 may be formed from materials that are suited to self-expansion and delivery using a self-expandable implant delivery system. For example, implant 500 may be formed from superelastic nickel titanium alloys. Alternatively, an implant 500 may be formed from plastically deformable polymers and self-expandable polymers, such as various formulations of polyurethane and polyethylene.

Implant 500 may be fabricated using manufacturing processes that are known in the field of stent manufacturing. For example, balloon expandable or self-expandable aneurysm implants 500 having a structure described in the embodiments above may be laser cut from raw material tubing. In an embodiment, raw Nitinol tubing with an outer diameter of 0.081-inch and a wall thickness of 0.004-inch may be used. Laser cutting may be followed by a combination of cleaning, polishing, and passivation processes. For example, in the case of balloon expandable implants 500, the implant 500 may be etched, passivated, and/or electropolished to achieve a surface finish that is clean, atraumatic to vessel tissue, and corrosion resistant. In the case of self-expandable implants 500, the implant 500 may be sand-blasted, etched, electropolished, and passivated to achieve a suitable surface finish.

In addition to finishing the surface of implant 500, various steps may be followed to modify the implant 500 configuration. For example, various heat treatment steps may be applied to a self-expandable implant 500 in order to provide a heat set material memory in the fully expanded configuration. Heat setting may involve expansion of base section 502 and aneurysm section 504 to the desired configuration using a sequence of heat treating steps. For example, base section 502 and aneurysm section 504 may be placed over a mandrel of a desired diameter in each step to sequentially increase the diameter to a deployment diameter, e.g., about 4.25 mm. Additionally, in an embodiment, aneurysm section 504 may be separately placed over a mandrel having a bulbous shape to heat set the aneurysm section 504 with a bulbous contour that may protrude into an aneurysm sac while base section 502 may be maintained in a cylindrical contour using corresponding cylindrical mandrels.

Implant 500 may be loaded onto or into a delivery system in numerous manners. For example, in the case of a balloon-expandable implant, a crimping process may reduce the diameter of a laser cut implant 500 to affix the implant struts to a non-compliant or semi-compliant balloon of a balloon delivery catheter. In the case of a self-expandable implant 500, one or more crimping processes may be applied to reduce the diameter of implant 500 until it may be loaded into a delivery sheath of a self-expandable delivery system that constrains implant 500 during delivery. In an embodiment, a two stage crimping process may be used. For example, a first stage may crimp the implant 500 to a cylindrical configuration in which aneurysm section 504, which may initially be bulbous, is in a stacked state and base section 502 is in an expanded state. A second crimping stage may follow, in which the implant 500 is crimped to a final cylindrical configuration with aneurysm section 504 and base section 502 both in a stacked state.

These and other processes may be performed in accordance with skill in the art. For example, coating processes may be used to coat the implant surface with therapeutic agents, including drugs that have been used in the field of drug-eluting stents, e.g., paclitaxel, zotarolimus, everolimus, sirolimus, etc. These agents may be used alone or in combination with polymer carriers, such as biostable or biodegradable polymers that may be loaded to retain and time-release a therapeutic agent. Thus, the manufacturing processes provided above are illustrative and not limiting of the range of manufacturing processes that may be used to form an implant 500 and to prepare the implant for delivery to an aneurysm location within a patient.

FIG. 23 illustrates a pictorial view of an intravascular access path to an aneurysm site in a patient. An aneurysm in a patient vessel may be accessed through various locations, including a femoral access site 2300 or a radial access site 2302. For example, an intravascular path 2304 may be accessed through those locations using an introducer kit and a guidewire, as is well known. Intravascular path 2304 may then be followed using the guidewire until an aneurysm site 2306 is reached. For example, aneurysm site 2306 may be accessed in a cerebral vessel by a guidewire tracked from femoral access site 2300 through a femoral artery, aorta, carotid artery, and various cerebral vessels of intravascular path 2304.

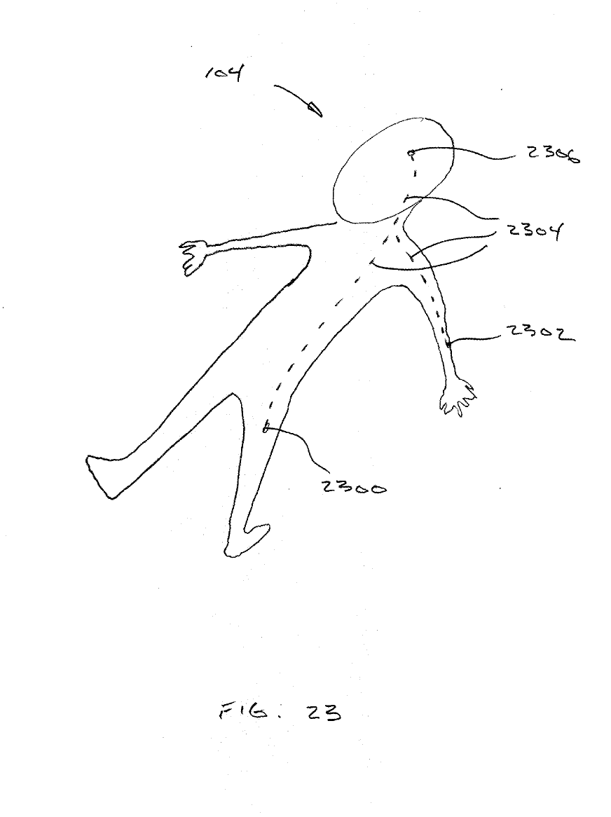

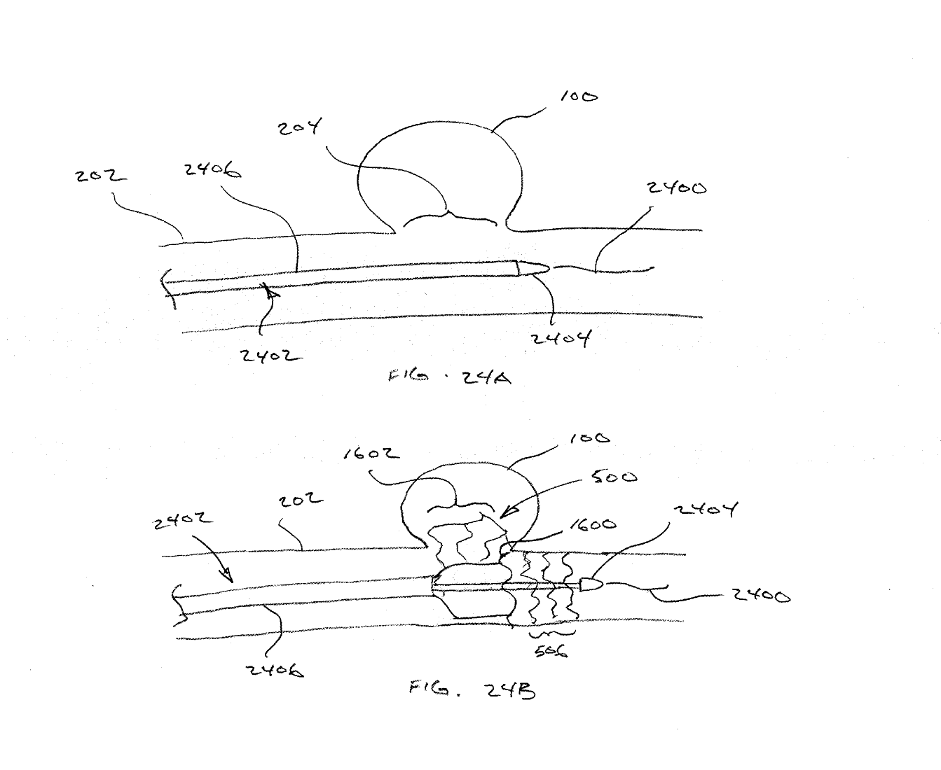

Referring to FIG. 24A, a pictorial view of a delivery system being tracked to an aneurysm site is shown in accordance with an embodiment of the invention. One skilled in the art will recognize that the system illustrated in FIG. 24A may include a construction similar to other delivery systems 2402 used for delivering a self-expandable stent into a patient vasculature. However, delivery of a balloon expandable implant 500 may be achieved using balloon expandable delivery systems 2402, as is also known in the art. After aneurysm site 2306 is accessed by a guidewire 2400, a delivery system 2402 may be delivered over guidewire 2400 until a distal tip 2404 of delivery system 2402 is in the vicinity of aneurysm site 2306, e.g., distal to aneurysm gate 204. Delivery system 2402 may include outer sheath 2406 to constrain a crimped implant 500 to a delivery diameter. Longitudinal placement of delivery system 2402 may be visualized and controlled according to end markers 1204 and aneurysm markers 2002 that are viewed under, e.g., fluoroscopy. In addition, delivery system 2402 may have markers both distal and proximal to the implant 500 so that the relative position between delivery system 2402 and implant 500 may be visualized. More specifically, aneurysm markers 2002 may be longitudinally aligned with aneurysm gate 204 by advancing or retracting delivery system 2402 over guidewire 2400. Similarly, aneurysm markers 2002 may be circumferentially aligned relative to aneurysm gate 204, e.g., by orienting aneurysm markers 2002 near lateral apex 1102 of aneurysm section holders 514 furthest from aneurysm 100 in parent vessel 202. Aneurysm markers 2002 may be aligned in this manner to ensure that aneurysm pattern 516 apposes aneurysm gate 204 when implant 500 is deployed from delivery system 2402.

Referring to FIG. 24B, a pictorial view of a vascular implant partially deployed from a delivery system at an aneurysm site is shown in accordance with an embodiment of the invention. After implant 500 is aligned and positioned relative to aneurysm 100, implant 500 may be deployed into parent vessel 202. In the case of a self-expandable implant 500, outer sheath 2406 may be retracted from distal tip 2404. Thus, distal base subsection 506 and end arc 1600 may expand within parent vessel 202 and medial arcs 1602 may expand into or against aneurysm gate 204. In an embodiment, for example—but not necessarily—when aneurysm pattern 516 includes a zig-zag pattern, aneurysm pattern 516 may remain flush with aneurysm gate 204, i.e., aneurysm pattern 516 may assume a longitudinal cylindrical segment contour. In an alternative embodiment, for example—but not necessarily—when aneurysm pattern 516 includes a parallelogram pattern, aneurysm pattern 516 may protrude into aneurysm sac 200, i.e., aneurysm pattern 516 may assume a bulbous contour. It is to be understood that both zig-zag and parallelogram aneurysm sections may be expanded to be flush with aneurysm gate 204 or protruding into aneurysm sac 200. Thus, the examples above are not restrictive. In a case of a balloon expandable implant 500, expansion from an unexpanded state to an expanded state may be facilitated by introducing an inflation fluid into a balloon that applies an outward force on implant 500 and causes implant 500 to increase in diameter until it expands into or against parent vessel 202 and aneurysm 100.

Referring to FIG. 24C, a pictorial view of a vascular implant fully deployed from a delivery system at an aneurysm site is shown in accordance with an embodiment of the invention. Outer sheath 2406 may be retracted further to fully deploy implant 500 at aneurysm site 2306. Fully deployed implant 500 includes distal and proximal base subsections 506, 508 in apposition with parent vessel 202. Thus, base section 502 anchors implant 500 at aneurysm site 2306 and provides radial scaffolding to parent vessel 202. Furthermore, aneurysm section 504 deploys across an aneurysm segment of parent vessel 202 such that aneurysm pattern 516 apposes aneurysm gate 204. Aneurysm section holders 514 may appose parent vessel 202 on either side of aneurysm gate 204 to form a tight seal with parent vessel 202 and to restrict blood flow into aneurysm gate 204 to blood passing through aneurysm pattern 516. In some embodiments, portions of aneurysm pattern 516, such as medial arcs 1602, may protrude into aneurysm sac 200. Furthermore, aneurysm pattern 516 may scaffold aneurysm gate 204 more densely, i.e., with a higher ratio of scaffolding coverage to total surface area, than parent vessel 202 is scaffolded by base section 502.

Protrusion into aneurysm sac 200 may occur automatically in response to deployment in some embodiments. For example, in the case of a self-expanding implant 500 that was processed using a bulbous mandrel to heat set aneurysm pattern 516 in a bulbous contour, aneurysm pattern 516 may naturally expand toward a bulbous shape as it is released from outer sheath 2406 into the patient anatomy. However, in other embodiments, secondary deployment steps may be required to achieve a bulbous contour in aneurysm pattern 516. For example, an angioplasty balloon catheter may be secondarily tracked over guidewire 2400 after removing delivery system 2402, and a balloon of the secondary catheter may be expanded to plastically deform aneurysm pattern 516 outward beyond an initial cylindrical segment deployment diameter. This technique may be used to achieve a bulbous aneurysm pattern contour in either a balloon expandable implant or a self-expandable implant.

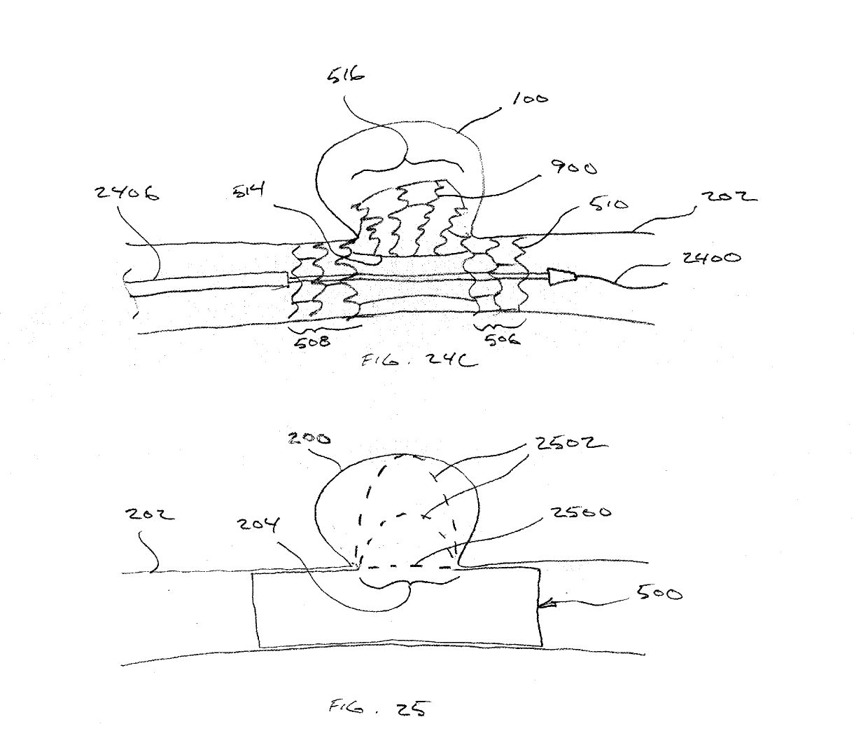

Referring to FIG. 25, a schematic view showing a plurality of possible contours of an aneurysm pattern of a vascular implant deployed at an aneurysm site is shown in accordance with an embodiment of the invention. As described above, aneurysm pattern 516 may be designed to expand into various contours. For example, aneurysm pattern 516 may expand toward a longitudinal cylindrical segment contour 2500. Cylindrical segment contour 2500 of aneurysm pattern 516 may be collinear with parent vessel 202. More specifically, the contour of aneurysm pattern 516 deployed in cylindrical segment contour 2500 may effectively bridge the cylindrical form of parent vessel 202 across aneurysm gate 204. Alternatively, aneurysm pattern 516 may expand toward one or more bulbous contours 2502 that protrude into aneurysm sac 200 to varying depths. The depth of protrusion into aneurysm sac 200 may be controlled prior to deployment through design of aneurysm pattern 516 and/or after deployment using secondary balloon inflations to force aneurysm pattern 516 into aneurysm sac 200, as described above. Bulbous contours 2502 may appose an aneurysm wall directly or may leave a gap between the aneurysm wall and the aneurysm pattern 516.

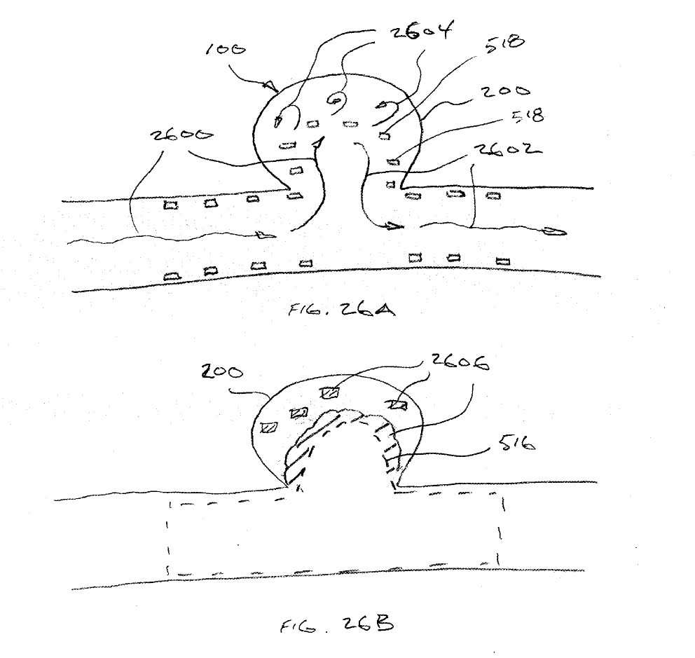

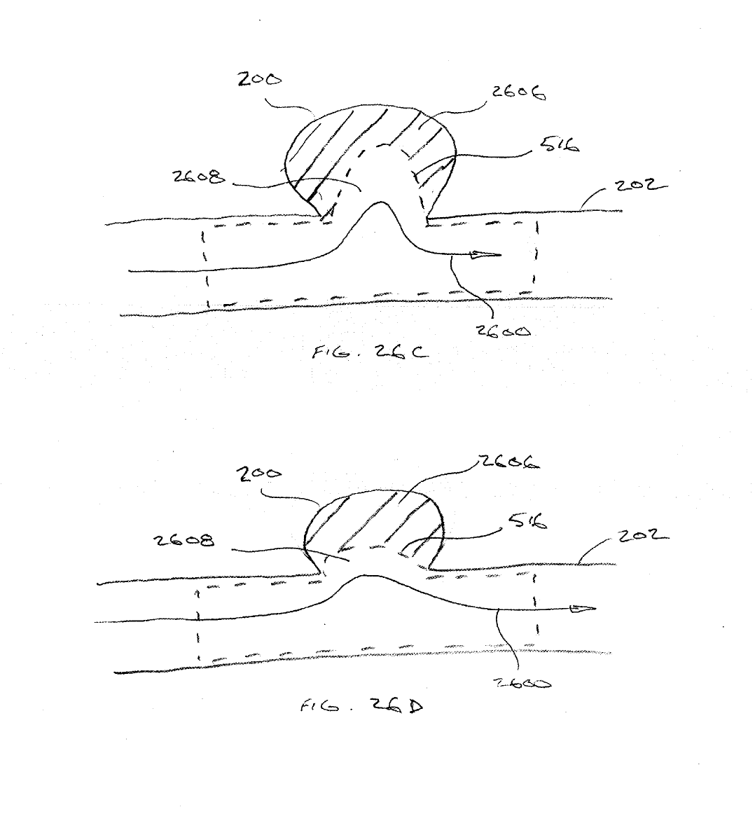

Referring to FIG. 26A, a pictorial view of a vascular implant deployed at an aneurysm site and diverting blood flow into and away from an aneurysm is shown in accordance with an embodiment of the invention. In an embodiment, implant 500 is deployed at aneurysm site 2306 with base section 502 scaffolding parent vessel 202 and aneurysm pattern 516 scaffolding aneurysm 100 across aneurysm gate 204. In an embodiment, aneurysm pattern 516 may be flush with aneurysm gate 204 and parent vessel 202. However, in an alternative embodiment as illustrated in FIG. 26A, aneurysm pattern 516 may self-expand into aneurysm sac 200 and or be plastically deformed into aneurysm sac 200 by a secondary compliant, semi-compliant, or non-compliant balloon. When aneurysm pattern 516 protrudes into aneurysm sac 200, blood inflow 2600 through parent vessel 202 may flow through aneurysm gate 204 toward aneurysm pattern 516. As inflow 2600 meets aneurysm pattern 516, it may be diverted by the aneurysm arc segments 518. More specifically, some blood flow will be diverted back toward parent vessel 202 as outflow 2602. Outflow 2602 will continue downstream unmodified from its initial state. The amount of blood diverted to outflow 2602 may be greater for aneurysm pattern 516 with a cylindrical segment contour 2500 than for aneurysm pattern 516 with a bulbous contour 2502, since aneurysm pattern 516 may be more densely packed in a cylindrical segment configuration, and thus permit less blood to pass through the pattern. However, a portion of inflow 2600 may pass through aneurysm pattern 516 into a gap between aneurysm pattern 516 and aneurysm sac 200.

In an embodiment, aneurysm pattern 516 facilitates depressurization of an aneurysm 100 in one or more ways. First, aneurysm struts of aneurysm pattern 516 may be relatively narrow compared to, e.g., base struts of base section 502. For example, aneurysm struts may have a strut width of about 0.0012-inch while base strut width may be about 0.002-inch. Accordingly, the relatively smaller aneurysm strut widths may facilitate and promote faster clotting on aneurysm pattern 516 as compared to base section 502. Thus, as blood flow is diverted by aneurysm pattern 516, aneurysm pattern 516 may begin to become covered by blood clotting at a greater rate across aneurysm gate 204 than other areas of implant 500 are covered within other regions of parent vessel 202. Second, a portion of flow may be slowed significantly as it passes through aneurysm pattern 516, creating laminar Eddy currents 2604. Thus, blood flowing into a gap between aneurysm pattern 516 and aneurysm wall may clot quickly to fill the gap.

Referring to FIG. 26B, a pictorial view of a vascular implant deployed at a site of a partially embolized aneurysm is shown in accordance with an embodiment of the invention. Eddy currents laminar flow, i.e., swirling low speed flow, and stagnated flow within aneurysm sac 200 gap and on aneurysm pattern 516 may accelerate and promote clotting processes in blood, causing embolized blood 2606 and blood clots to form on aneurysm pattern 516 and within the aneurysm gap. For example, clotting may cover aneurysm arcs 518, resulting in a reduction in surface area exposed between the arcs. As aneurysm pattern 516 becomes covered, flow through aneurysm pattern 516 into aneurysm sac 200 will be further reduced until aneurysm pattern 516 closes. Closure of aneurysm pattern 516 reduces pressure on aneurysm sac 200 and prevents rupture of aneurysm wall.

Referring to FIG. 26C, a pictorial view of a vascular implant deployed at a site of a partially de-pressurized aneurysm is shown in accordance with an embodiment of the invention. As aneurysm pattern 516 becomes entirely occluded by clotted blood, the gap between aneurysm sac 200 and aneurysm pattern 516 may begin to simultaneously fill with clotted blood. Any remaining non-coagulated blood within gap will eventually become embolized since it will remain stagnant. Thus, aneurysm 100 will be fully embolized and blood inflow 2600 will be diverted by aneurysm pattern 516 toward parent vessel 202. Since aneurysm sac 200 and aneurysm pattern 516 are simultaneously embolized, a single device may be used to replace a combination of embolic coils and stents.

As described earlier, although implant 500 may be used alone to treat an aneurysm, under some conditions, it may also be used in combination with an embolic coil. For example, implant 500 may be used to jail an embolic coil placed within an aneurysm sac prior to implant deployment. Alternatively, implant 500 may be deployed and then an embolic coil may be inserted through aneurysm pattern into an aneurysm sac. The decision of whether and when to insert an embolic coil may be driven by best practices in some cases, and in other cases it may be driven by an assessment of how dense the aneurysm pattern appears across an aneurysm gate after deployment.

In an embodiment, a protrusion of aneurysm pattern 516 into aneurysm sac 200 may result in a recess 2608 outside of the cylindrical geometry of parent vessel 202. Therefore, flow characteristics within the recess 2608 may differ from those in parent vessel 202. More specifically, flow in recess 2608 may be slower than in parent vessel 202 to promote clotting. Furthermore, an increased density and a decreased strut width of struts in aneurysm section 504 as compared to struts in base section 502 may promote faster clotting on aneurysm section 504. Accordingly, the administration of blood thinning agents may affect blood clotting on aneurysm pattern 516 differently from clotting on base section 502. More specifically, while blood thinning agents may prevent blood from clotting on base section 502, Eddy currents 2604 and blood slowing within the recess 2608, as well as the propensity of aneurysm pattern 516 to activate clotting, may generate blood clotting on aneurysm pattern 516 despite the use of blood thinners. Therefore, aneurysm pattern 516 may create clotting on aneurysm section 504 and within aneurysm sac 200 independently of base section 502, even when blood thinners are administered.

Referring to FIG. 26D, a pictorial view of a vascular implant deployed at a site of a fully de-pressurized aneurysm is shown in accordance with an embodiment of the invention. In an embodiment, as embolized blood 2606 clots within aneurysm sac 200 and over aneurysm pattern 516, pressure within aneurysm sac 200 may be correspondingly reduced. For example, given that inflow 2600 is slowly diverted from aneurysm sac 200, pressure induced by blood flow may be reduced. Accordingly, as pressure within aneurysm sac 200 is reduced, aneurysm sac 200 may decrease in size. This shrinking may occur in combination with blood clotting over aneurysm pattern 516, and thus, aneurysm pattern 516 may also retract toward parent vessel 202, resulting in a less bulbous contour that more closely matches a parent vessel profile.

Although the deployment procedure has been described primarily in relation to an implant 500 with a bulbous aneurysm pattern contour, similar processes and results may be achieved with an aneurysm pattern 516 having a cylindrical segment contour. More specifically, following deployment, blood clotting may occur within aneurysm sac 200 due to the induction of eddy currents 2604 by aneurysm pattern 516 having cylindrical segment contour 2500. Simultaneously, blood clotting may occur over aneurysm pattern 516, gradually slowing the blood flow into aneurysm sac 200 and depressurizing the aneurysm sac 200. Eventually, aneurysm pattern 516 may become completely occluded and any remaining blood within aneurysm sac 200 will also clot. Thus, a single device may be used to replace the combination of an embolic coil and a stent. However, in some cases as described above, implant 500 may be used in combination with an embolic coil placed before or after deployment of implant 500 across an aneurysm gate.

In the foregoing specification, the invention has been described with reference to specific exemplary embodiments thereof. It will be evident that various modifications may be made thereto without departing from the broader spirit and scope of the invention as set forth in the following claims. The specification and drawings are, accordingly, to be regarded in an illustrative sense rather than a restrictive sense.

What is claimed is:

1. A vascular implant having an unexpanded state and an expanded state, the vascular implant comprising:
a base section having a plurality of base rings arranged along a longitudinal axis, the base section cylindrical in both the unexpanded state and the expanded state; and
an aneurysm section having a plurality of aneurysm section holders extending longitudinally from the base section and an aneurysm pattern radially disposed between the plurality of aneurysm section holders, the aneurysm pattern substantially cylindrical in the unexpanded state and substantially non-cylindrical in the expanded state.

2. The vascular implant of claim 1, wherein the aneurysm pattern includes a plurality of aneurysm arcs extending radially between the plurality of aneurysm section holders, and wherein one or more of the aneurysm arcs extend along a substantially circumferential path in the unexpanded state and extend along a substantially non-circumferential path in the expanded state.

3. The vascular implant of claim 1, wherein the base section further includes a proximal subsection and a distal subsection, and wherein the plurality of aneurysm section holders extend longitudinally between the proximal subsection and the distal subsection.

4. The vascular implant of claim 3, wherein the aneurysm section further includes an aneurysm connector extending longitudinally between the proximal subsection and the distal subsection, the aneurysm connector opposite of the plurality of aneurysm section holders from the aneurysm pattern.

5. The vascular implant of claim 4, further comprising one or more aneurysm marker holders in each of the plurality of aneurysm section holders, and an aneurysm marker in each aneurysm marker holder.

6. The vascular implant of claim 5, wherein the one or more aneurysm marker holders are longitudinally spaced along respective aneurysm section holders, and wherein the aneurysm markers include radiopaque markers.

7. The vascular implant of claim 5, wherein each base ring includes a plurality of base struts interconnected by a plurality of base joints and arranged in a ring pattern.

8. The vascular implant of claim 7, wherein each base strut extends straightly between a respective pair of base joints.

9. The vascular implant of claim 7, wherein each base strut undulates between a respective pair of base joints.

10. The vascular implant of claim 9, wherein the ring pattern of a first base ring includes a sawtooth pattern, wherein the ring pattern of a second base ring adjacent to the first base ring includes the sawtooth pattern, and wherein the sawtooth pattern of the second base ring is inverted relative to the sawtooth pattern of the first base ring.

11. The vascular implant of claim 7, wherein a plurality of base ring connectors interconnect adjacent base rings.

12. The vascular implant of claim 11, wherein the plurality of base ring connectors interconnect adjacent base rings at radially staggered locations along a substantially helical path.

13. The vascular implant of claim 11, wherein the plurality of base rings includes a transition ring connected to the aneurysm section, wherein the transition ring interconnects an adjacent base ring with the plurality of aneurysm section holders, and wherein the transition ring includes more base joints than the adjacent base ring.

14. The vascular implant of claim 13, wherein a transition ring connector interconnects the transition ring with the adjacent base ring, and wherein the plurality of aneurysm section holders extend longitudinally from a plurality of base joints of the transition ring.

15. The vascular implant of claim 1, wherein the aneurysm pattern includes a substantially bulbous contour in the expanded state.

16. The vascular implant of claim 1, wherein the aneurysm pattern includes a substantially longitudinal cylindrical segment contour in the expanded state.

17. A method, comprising:
advancing, into a vessel segment in an unexpanded state, a vascular implant including a base section having a plurality of base rings, and an aneurysm section having a plurality of aneurysm section holders extending longitudinally from the base section and an aneurysm pattern radially disposed between the plurality of aneurysm section holders, the base section and the aneurysm pattern substantially cylindrical in the unexpanded state;
deploying the vascular implant to an expanded state within the vessel segment at a site of an aneurysm, the aneurysm having an aneurysm sac adjoined to the vessel segment at an aneurysm gate, the base section substantially cylindrical and the aneurysm pattern substantially non-cylindrical in the expanded state.

18. The method of claim 17, wherein the aneurysm pattern includes a substantially bulbous contour bulging into the aneurysm sac in the expanded state.

19. The method of claim 17, wherein the aneurysm pattern includes a substantially longitudinal cylindrical segment contour collinear with a vessel wall of the vessel segment in the expanded state.

20. The method of claim 17, further comprising aligning one or more aneurysm markers in each of the plurality of aneurysm section holders with the aneurysm gate.
